(19) United States
(12) Patent Application Publication
(10) Pub. No.: US 2022/0162188 A1
(43) Pub. Date: May 26, 2022

(54) ISOPRENOIDS AND METHODS OF MAKING THEREOF

(71) Applicant: NORTH CAROLINA STATE UNIVERSITY, Raleigh, NC (US)

(72) Inventors: Gavin WILLIAMS, Raleigh, NC (US); Sean LUND, Raleigh, NC (US); Rachael HALL, Raleigh, NC (US)

(21) Appl. No.: 17/423,316

(22) PCT Filed: Jan. 15, 2020
(86) PCT No.: PCT/US2020/013663

§ 371 Date: Jul. 15, 2021

Related U.S. Application Data
(60) Provisional application No. 62/792,523, filed on Jan. 15, 2019.

(51) Int. Cl. C07D 403/06; C07D 409/14; C07D 401/14; C07D 405/14; C07D 403/14; C12P 17/16

(52) U.S. Cl. CPC C07D 403/06; C07D 409/14; C12P 17/165; C07D 405/14; C07D 403/14; C07D 401/14

(57) ABSTRACT

Disclosed are methods for preparing isoprenoid subunits, as well as methods of employing these isoprenoid subunits for the synthesis of isoprenoids. Also provided are isoprenoids prepared using the methods described herein.

### CROSS REFERENCE TO RELATED APPLICATIONS

This application claims benefit of U.S. Provisional Application No. 62/792,523, filed Jan. 15, 2019, which is hereby incorporated by reference in its entirety.

### STATEMENT REGARDING FEDERALLY SPONSORED RESEARCH OR DEVELOPMENT

This invention was made with Government Support under Grant No. 556943 awarded by the National Institutes of Health. The Government has certain rights in the invention.

### BACKGROUND

Isoprenoids comprise >55,000 natural products for which methods to access and diversify their structures are in high demand. Ultimately, the isoprene motif plays a critical role in modulating the biological activity of isoprenoids, determines their utility as tools to study and treat human diseases, and provides the basis to develop new fuels and chemicals. Notably, although several valuable isoprenoids have been accessed via heterologous expression, the ability to diversify isoprenoids is extremely limited largely due to critical limitations imposed by native isoprenoid biosynthesis.

Firstly, only the mevalonate (MEV) and 1-deoxy-D-xylulose-5-phosphate (DXP) pathways (FIG. 15A) are known to produce the universal hemiterpene isoprenoid diphosphate building blocks, dimethylallyl pyrophosphate (DMAPP) and isopentenyl pyrophosphate (IPP). The negatively charged hemiterpenes are not cell permeable, thus preventing feeding them or analogues thereof into cultures. The MEV and DXP pathways involve at least six enzymatic steps, each with stringent substrate specificity and therefore offer little opportunity to diversify the structures of isoprenoids through feeding in non-natural precursors. As a result, while precursor-directed biosynthesis has proven a powerful approach to access diverse structures of natural products-especially polyketides—by feeding non-natural building blocks, this approach has not yet been applied to isoprenoids. Furthermore, late-stage biosynthetic modification of isoprenoid scaffolds is typically limited to oxidations, often catalyzed by P450's.

Secondly, terpene metabolism is highly regulated and is a burden to the carbon supply on the cell. For example, the MEV pathway uses three molecules of phosphate donor (ATP) and two reducing equivalents (NADPH) for each DMAPP/IPP, while the DXP pathway requires two phosphate donors (ATP and CTP) and two reducing equivalents (NADPH) (FIG. 15A).

Thirdly, given that native terpenes are typically essential for maintenance of the cell, genetic modification of native hemiterpene pathways would likely be lethal.

Accordingly, new methods of making isoprenoids are needed.

### SUMMARY

Together, the limitations associated preparing isoprenoid derivatives utilizing existing biochemical pathways can be overcome by supplying a membrane-permeable carbon building block dedicated for a designer pathway that would function independent of native isoprenoid metabolism. A potential strategy for hemiterpene biosynthesis can start with an alcohol (e.g., isopentenol (ISO) and/or dimethylallyl alcohol (DMAA)), which can be converted to a pyrophosphate (diphosphate) via stepwise enzymatically catalyzed phosphorylation (see, for example, FIG. 15B). These alcohol-dependent hemiterpene (ADH) pathways can employ two independent kinases (a first kinase and a second kinase), a phosphatase that exhibits bidirectional activity and a kinase, or a single enzyme that can catalyze both a first phosphorylation and a second phosphorylation of the primary alcohol (e.g., a phosphotransferase). These pathways are completely orthogonal to the endogenous DMAPP/IPP biosynthetic machinery, such that non-natural precursors are not expected to inhibit endogenous enzymatic machinery. In addition, these routes only utilize two equivalents of ATP. Furthermore, these artificial pathways designed bottom-up as a replacement for natural hemiterpene biosynthesis can leverage naturally or engineered promiscuous enzymes that enable a broad panel of easily scalable and accessible alcohols to be converted to the corresponding diphosphate. These diphosphates can then be introduced into downstream natural or artificial isoprenoid biosynthetic pathways. In this way, a wide variety of isoprenoids can be biosynthetically prepared, including isoprenoids that cannot be readily prepared using conventional synthetic means.

Accordingly, provided herein are methods for synthesizing an isoprenoid subunit. In some embodiments, these methods can comprise (i) contacting a primary alcohol defined by Formula I below

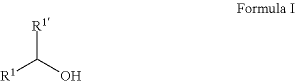

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; with a phosphatase that exhibits bidirectional activity in the presence of ATP to form a phosphate defined by Formula II below

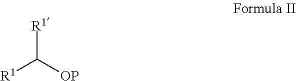

wherein R¹, R^{1′}, and R^{X} are as defined above with respect to Formula I an presents a phosphate group; and (ii) contacting the phosphate defined by Formula II with a kinase in the presence of ATP to generate the isoprenoid subunit defined by Formula III below

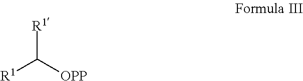

wherein R¹, R^{1′}, and R^{X} are as defined above with respect to Formula I and PP represents a pyrophosphate group.

In some embodiments, the phosphatase can comprise a non-specific acid phosphatase. In certain embodiments, the phosphatase can comprise PhoN.

In some embodiments, the kinase can comprise a kinase that uses a phosphate acceptor. For example, the kinase can be chosen from a polyphosphate kinase, a phosphomevalonate kinase, a phosphomethylpryimidine kinase, a farnesyl-diphosphate kinase, or a combination thereof. In certain embodiments, the kinase can comprise isopentenyl phosphate kinase (IPK). In some cases, the phosphatase, the kinase, or a combination thereof can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

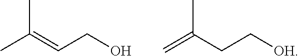

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the phosphatase that exhibits bidirectional activity and the kinase. The cell can be engineered to express (or overexpress) the genes encoding for the phosphatase and the kinase.

Methods can further comprise introducing the isoprenoid subunit into a natural or artificial isoprenoid biosynthetic pathway to synthesize an isoprenoid. This can be performed within a cell or in a cell-free system.

Also provided are methods for synthesizing an isoprenoid subunit that comprise (i) providing a cell comprising genes encoding for (1) a phosphatase that exhibits bidirectional activity, and (2) a kinase; and (ii) incubating the cell in a fermentation broth with ATP and a primary alcohol defined by Formula I below

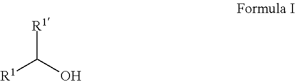

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; thereby generating the isoprenoid subunit.

In some embodiments, the phosphatase can comprise a non-specific acid phosphatase. In certain embodiments, the phosphatase can comprise PhoN.

In some embodiments, the kinase can comprise a kinase that uses a phosphate acceptor. For example, the kinase can be chosen from a polyphosphate kinase, a phosphomevalonate kinase, a phosphomethylpryimidine kinase, a farnesyl-diphosphate kinase, or a combination thereof. In certain embodiments, the kinase can comprise isopentenyl phosphate kinase (IPK). In some cases, the phosphatase, the kinase, or a combination thereof can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

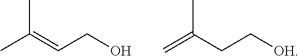

Methods can further comprise introducing the isoprenoid subunit into a natural or artificial isoprenoid biosynthetic pathway to synthesize an isoprenoid, and isolating the resulting isoprenoid.

Also provided are methods for synthesizing an isoprenoid subunit that comprise (i) contacting a primary alcohol defined by Formula I below

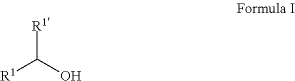

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with the proviso that the primary alcohol defined by Formula I is not one of the following

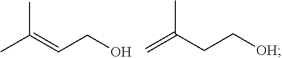

with a first kinase in the presence of ATP to form a phosphate defined by Formula II below

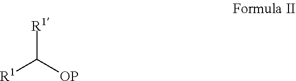

wherein R¹, R^{1′}, and R^{X} are as defined above with respect to Formula I and P represents a phosphate group; and (ii) contacting the phosphate defined by Formula II with a second kinase in the presence of ATP to generate the isoprenoid subunit defined by Formula III below

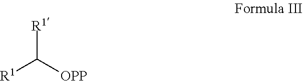

wherein R¹, R^{1′}, and R^{X} are as defined above with respect to Formula I and PP represents a pyrophosphate group.

In some embodiments, the first kinase can comprise a kinase that uses an alcohol acceptor. For example, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. For example, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In certain embodiments, the first kinase, the second kinase, or a combination thereof comprise a mutant enzyme engineered to increase substrate promiscuity, improve enzyme activity, increase enzyme specificity with respect to a particular substrate, or a combination thereof.

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) providing a cell comprising genes encoding for a first kinase and a second kinase; (ii) incubating the cell in a fermentation broth with ATP and a primary alcohol defined by Formula I below

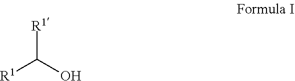

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with the proviso that the primary alcohol defined by Formula I is not one of the following

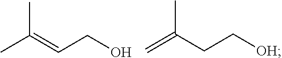

thereby generating the isoprenoid subunit.

In some embodiments, the first kinase can comprise a kinase that uses an alcohol acceptor. For example, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. For example, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In certain embodiments, the first kinase, the second kinase, or a combination thereof comprise a mutant enzyme engineered to increase substrate promiscuity, improve enzyme activity, increase enzyme specificity with respect to a particular substrate, or a combination thereof.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) contacting a primary alcohol defined by Formula I below

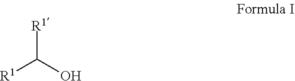

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with a first kinase in the presence of ATP to form a phosphate defined by Formula II below

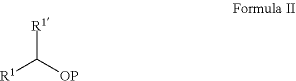

wherein R¹, R^{1′}, and R^{X} are as defined above with respect to Formula I and P represents a phosphate group; and (ii) contacting the phosphate defined by Formula II with a second kinase in the presence of ATP to generate the isoprenoid subunit defined by Formula III below

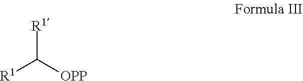

wherein R¹, R^{1′}, and R^{X} are as defined above with respect to Formula I and PP represents a pyrophosphate group; wherein the first kinase, the second kinase, or a combination thereof comprise a mutant enzyme engineered to increase substrate promiscuity, improve enzyme activity, increase enzyme specificity with respect to a particular substrate, or a combination thereof.

In some embodiments, the first kinase can comprise a kinase that uses an alcohol acceptor. For example, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. For example, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In some embodiments, the primary alcohol defined by Formula I is not one of the following

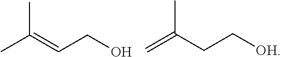

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) providing a cell comprising genes encoding for a first kinase and a second kinase, wherein the first kinase, the second kinase, or a combination thereof comprise a mutant enzyme engineered to increase substrate promiscuity, improve enzyme activity, increase enzyme specificity with respect to a particular substrate, or a combination thereof; (ii) incubating the cell in a fermentation broth with ATP and a primary alcohol defined by Formula I below

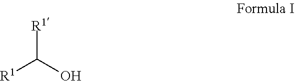

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; thereby generating the isoprenoid subunit.

In some embodiments, the first kinase can comprise a kinase that uses an alcohol acceptor. For example, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. For example, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In some embodiments, the primary alcohol defined by Formula I is not one of the following

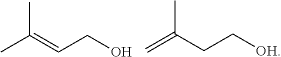

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) contacting a primary alcohol defined by Formula I below

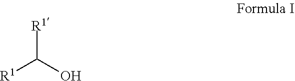

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; with a single enzyme in the presence of ATP to generate the isoprenoid subunit defined by Formula III below

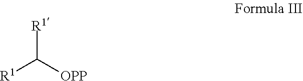

wherein R¹, R^{1′}, and R^{X} are as defined above with respect to Formula I and PP represents a pyrophosphate group, wherein the single enzyme comprises a phosphotransferase that can catalyze both a first phosphorylation and a second phosphorylation of the primary alcohol defined by Formula I to generate the isoprenoid subunit defined by Formula III.

In some embodiments, the single enzyme can comprise a phosphotransferase that uses an alcohol acceptor. In some embodiments, the single enzyme can comprise a phosphotransferase that uses a phosphate acceptor. In some embodiments, the single enzyme can comprise isopentenyl phosphate kinase (IPK). In certain embodiments, the single enzyme can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

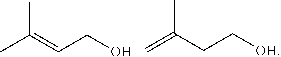

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) incubating a cell in a fermentation broth with ATP and a primary alcohol defined by Formula I below

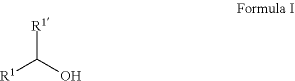

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, thereby generating the isoprenoid subunit defined by Formula III below

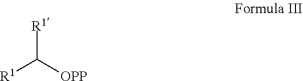

wherein R¹, R^{1′}, and R^{X} are as defined above with respect to Formula I and PP represents a pyrophosphate group; wherein the cell comprises a gene encoding for a phosphotransferase that can catalyze both a first phosphorylation and a second phosphorylation of the primary alcohol defined by Formula I to generate the isoprenoid subunit.

In some embodiments, the single enzyme can comprise a phosphotransferase that uses an alcohol acceptor. In some embodiments, the single enzyme can comprise a phosphotransferase that uses a phosphate acceptor. In some embodiments, the single enzyme can comprise isopentenyl phosphate kinase (IPK). In certain embodiments, the single enzyme can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

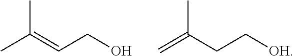

Methods can further comprise introducing the isoprenoid subunit into a natural or artificial isoprenoid biosynthetic pathway to synthesize an isoprenoid. This can be done performed within a cell or in a cell-free system.

As discussed above, the methods described herein can be used to prepare a variety of isoprenoids. Accordingly, provided herein are a variety of new isoprenoids, including isoprenoid defined by Formula IV below

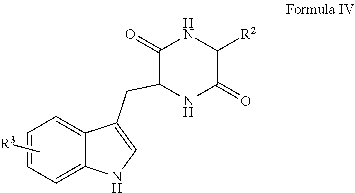

wherein R² is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R³ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R² is hydrogen. In other embodiments, R² can be selected from the group consisting of 6-10 membered aryl, 5-10 membered heteroaryl, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups. In certain embodiments, R² can comprise:

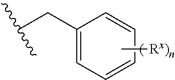

wherein n is 0, 1, or 2 and R^{X} is as defined above with respect to Formula IV.

In some embodiments, R³ is not one of the following

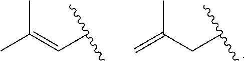

In some embodiments, R³ can be one of the following:

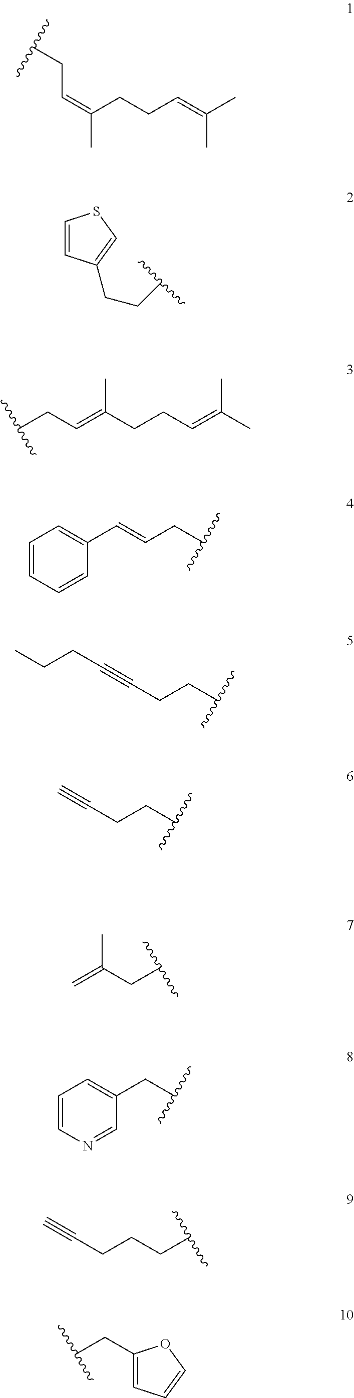

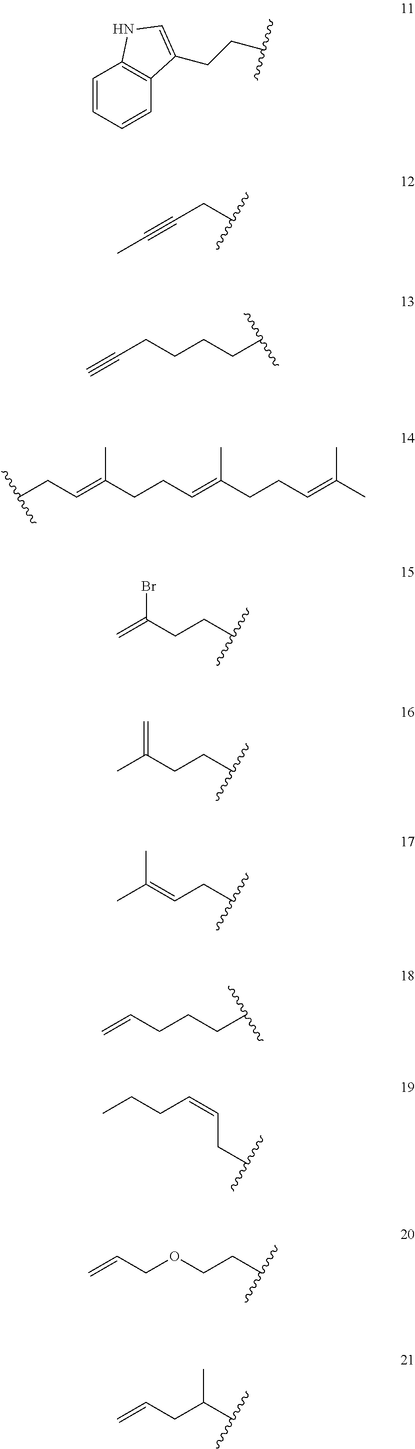

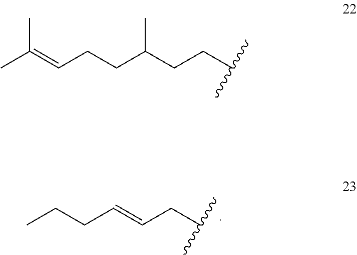

Also provided are isoprenoids defined by Formula V below

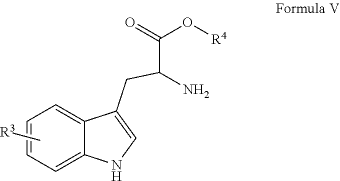

wherein R³ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R⁴ is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R⁴ can be hydrogen.

In some embodiments, R³ is not one of the following

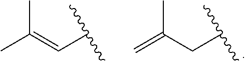

In some embodiments, R³ is one of the following:

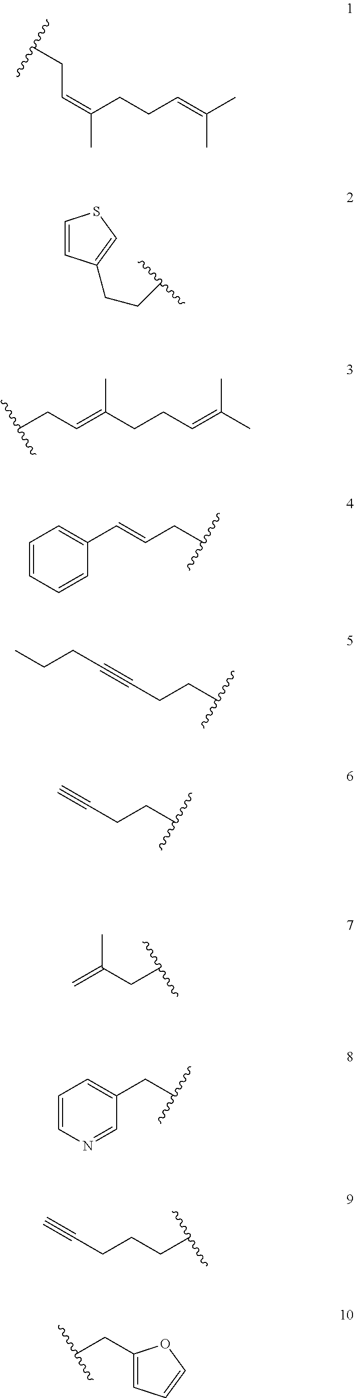

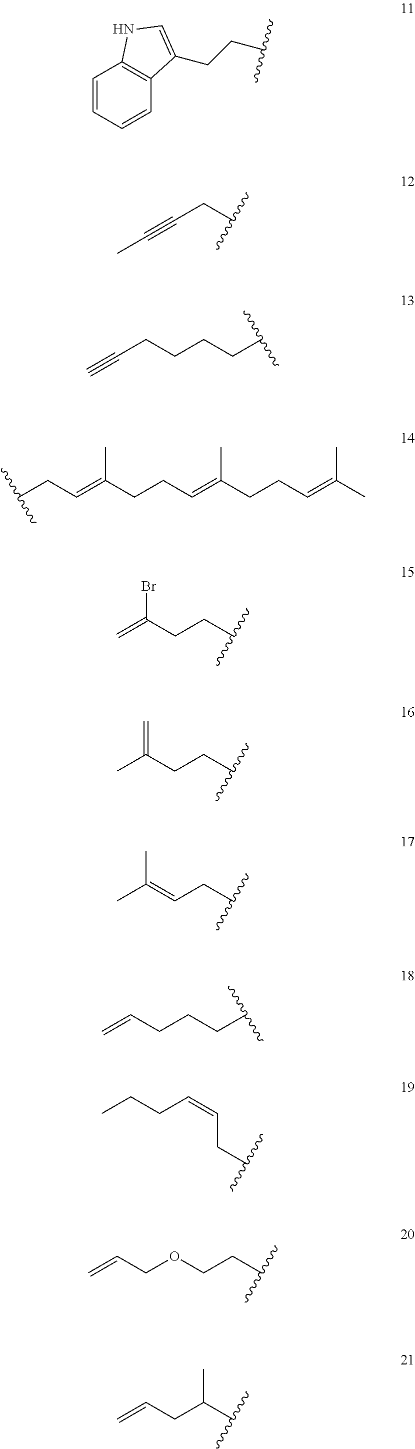

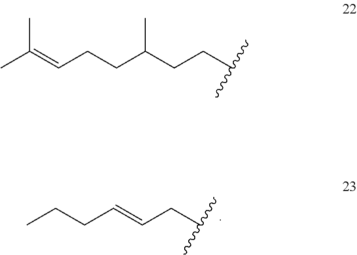

### DESCRIPTION OF DRAWINGS

FIG. 1 illustrates the structural diversity of terpenes. Terpene containing natural products have a variety of applications in fields such as agriculture (pyrethrin I), pharmaceuticals (Paclitaxel), and food (guaiol) among many others. Terpene natural products often are composed from linear portions of varying length prenyl groups that can be cyclized to a variety of ring structures such as the 7-membered ring in guaiol or the cyclopropyl group in cycloartenol. These prenyl groups can also be appended to natural products from other biosynthetic systems such as polyketide synthases as is seen in viridicatumtoxin.

FIG. 2 illustrates terpene biosynthesis from primary metabolism. In the mevalonate pathway, acetyl-CoA serves as the carbon source while in the DXP pathway, pyruvate and glyceraldehyde-3-phosphate (G3P) serve as carbon sources. These primary metabolites are converted to the common building blocks IPP and DMAPP before being assembled into linear precursor that can be cyclized into a wide variety of ring structures.

FIG. 3 illustrates the ability of prenyltransferases to generate diverse structures by the various combinations of hemiterpenes. Prenyltransferases utilize carbocationic chemistry to generate a wide-range of products. Head-to-middle prenyltransferases such as lavandulyl diphosphate synthase use two units of DMAPP. Head-to-head prenyltransferases use two units of DMAPP as is the case of chrysanthemyl diphosphate synthase. Head-to-tail prenyltransferases such as geranylpyrophosphate synthase use one DMAPP and one IPP and differential deprotonation events and resulting double bond isometry result in products such as geraniol or nerol.

FIG. 4 illustrates linear precursor generation and subsequent cyclization. Prenyltransferases catalyze carbon-carbon bond formation between five-carbon unit hemiterpenes to produce elongated substrates. The elongated substrates are then acted upon by terpene cyclases to generate a diverse array of products.

FIG. 5 illustrates a hypothetical cyclization of geranyl pyrophosphate. Terpene cyclases delicately direct various possible cyclization schemes afforded by the carbocationic chemistry available.

FIG. 6 illustrates a semi-synthetic route for the scalable production of artemisinin (antimalarial) through heterologous host engineering and chemical synthesis. To enable this process, S. cerevisiae was engineered to produce large amounts of the native metabolite IPP and express genes from Artemisia annua to produce high titers of artemisinic acid (25 g/L) which then served as a feedstock for the chemical synthesis of artemisin (45% overall yield from feedstock).

FIG. 7 shows the tailoring of terpenes afforded by biosynthesis. Terpene natural products can be hydroxylated or oxidized (colored in pink), isomerized (colored in blue), acylated (colored in orange), methylated (colored in purple), or appended to other classes of natural products (colored in green).

FIG. 8 shows the divergence of bioactivity by scaffold oxidation and modification.

FIG. 9 illustrates the direct diversification of plant extracts. Extracts were prepared that could be directly modified prior to chromatography using an existing synthetic handle in a repeatable fashion as judged by HPLC.

FIG. 10 shows a strategy of generating diversity from complexity. A ring distortion strategy can be used with gibberellic acid as a starting material to obtain a variety of other structures. These novel structures have a high proportion of sp3 carbons, stereocenters, and have a low c log P value indicating they could be decent compounds for chemical libraries.

FIG. 11 illustrates terpene diversity afforded through the use of substrate analogues. (A) The natural product of aristolochene synthase using farnesyl diphosphate. (B) Fluorination of C-2 in farnesyl diphosphate affords an intermediate product formed during the natural cyclization progression. (C) Use of an aniline analogue of farnesyl diphosphate by aristolochene synthase results in the formation of a 12-membered ring.

FIG. 12 illustrates the use of mutasynthesis for the production of fluorobalhimycin. An Amycolatopsis balhimycina mutant deficient for β-hydroxytyrosine biosynthesis allows for the incorporation of 3-fluoro-β-hydroxytyrosine. The natural product contains chlorine in place of fluorine. It is hypothesized that this halogenation event takes place late in the biosynthesis of this glycopeptide. Rather than engineer an alteration in substrate selectivity of the halogenase, alternative building blocks were supplied and the halogenation event prevented.

FIG. 13 illustrates a precursor-directed diversification strategy for the preparation of jadomycin analogues. Jadomycin derivatives can be prepared by supplementing the growth media with O-propargyl-L-serine as the sole nitrogen source. The resulting derivative can be used as a building block for copper(I)-catalyzed alkyne-azide cycloadditions (CuAAC) with a panel of azide functionalized sugars.

FIG. 14 illustrates biosynthetic building blocks and construction of natural products. R′ denotes either coenzyme-A or an acyl carrier protein linked unit. Polyketides are composed of malonyl coenzyme-A building blocks where R can be a variety of substituents including but not limited to hydrogen, methyl, ethyl, methoxy, and amino. Polyketides are generated by successive Claisen-like condensations. Non-ribosomal peptides are composed of amino acids that are linked together in an ATP dependent reaction to generate amide bonds. Terpenes have low fidelity in the extension of starter units and no diversity of building blocks exists naturally.

FIGS. 15A-15B illustrate natural and engineered hemiterpene biosynthetic pathways. FIG. 15A shows the MEV and DXP (grey box) pathways. A branch of the MEV pathway, the archaeal MEV I pathway, is shown in blue. FIG. 15B shows an artificial alcohol-dependent hemiterpene (ADH) pathway completely decoupled from native isoprenoid metabolism.

FIGS. 16A-16B illustrate a lycopene reporter system and preliminary characterization of IPK. As shown in FIG. 16A, the reporter system module generates lycopene from cellular hemiterpenes. Fs is expected to inhibit native hemiterpene biosynthesis via the DXP pathway but not an artificial alcohol-dependent hemiterpene pathway. As shown in FIG. 16B, E. coli BL21Tuner(DE3) harboring pCDFDuet-GGPP, pACYCDuet-Lyc, and pETDuet-IPK in wells of a microplate were treated with combinations of IPTG, DMAA/ISO, and Fs and visualized.

FIG. 17 shows the effect of fosmidomycin (Fs) on lycopene production in the absence of kinase overexpression. E. coli BL21Tuner(DE3) harboring pCDFDuet-GGPP, pACYCDuet-Lyc, and ‘empty’ pETDuet in wells of a microplate were treated with combinations of IPTG (to induce expression of heterologous genes), DMAA and ISO (potential substrates for hemiterpene production), and Fs (to knock-down DXP-dependent hemiterpene production). No DXP-independent lycopene production was observed when the cells were treated with IPTG, Fs, and provided DMAA/ISO, indicating that the activity of endogenous kinases were not sufficient to support lycopene production.

FIGS. 18A-18B illustrate lycopene titers supported by engineered E. coli strains. In FIG. 18A, lycopene titers are shown at 0, 12, 26, or 48 h post induction, in the presence or absence of DMAA/ISO, using E. coli NovaBlue(DE3) harboring pAC-LYCipi and the indicated plasmid with Fs treatment. Values are the average of three replicates. Error bars are the standard deviation. FIG. 18B shows the substrate dependence of lycopene titers determined at 0, 12, 26, or 40 h post induction with the strain harboring pETDuet-PhoN-IPK+pAC-LYCipi with Fs treatment. Values are the average of three replicates. Error bars are the standard deviation.

FIGS. 19A-19B show the extraction and quantification of lycopene. As shown in FIG. 19A, lycopene was extracted from E. coli Novablue(DE3) pAC-LYCipi and analyzed by HPLC. Lycopene eluted at 8.5 min. FIG. 19B shows a lycopene standard curve.

FIGS. 20A-20C show tryptophan prenylation via the artificial alcohol-dependent hemiterpene pathway. FIG. 20A shows the reaction scheme of the ADH pathway coupled to the prenyltransferase, FgaPT2. FIG. 20B shows HPLC chromatograms showing peaks corresponding to DMAT: (i) in vitro FgaPT2 reaction with synthetic DMAPP; (ii) in vitro reaction with purified PhoN, IPK, and FgaPT2; (iii) culture media from in vivo bioconversion with the ADH module. The chromatograms are scaled equally. FIG. 20C is an extracted-ion chromatogram showing a single peak corresponding to DMAT in the culture media of E. coli Rosetta(DE3)pLysS+pETDuet-PhoN-IPK+pCDFDuetFgaPT2 after treatment with DMAA/ISO, and IPTG.

FIG. 21 illustrates precursor directed natural product diversification strategies. The polyketide diversification platform hinges on the generation of unnatural malonyl-coenzyme A derivatives generated by the ATP-dependent ligation of various malonic acids and coenzyme A using a malonyl-CoA synthase (MatB) from Rhizobium trifolii. A terpene production platform that converts alcohols to their corresponding diphosphates is also being investigated.

FIG. 22 illustrates the substrate promiscuity of PhoN from S. flexneri. PhoN can phosphorylate a variety of primary alcohols (R) using various phosphate donors (D).

FIG. 23 illustrates the trichloroacetonitrile-promoted phosphorylation of various alcohols. A wide variety of alcohols were phosphorylated using this strategy to generate a diverse panel of substrates to test with IPK, as well as prenyltransferases, prenylelongases, and terperne synthases (e.g., IspA, FgaPT2, CpaD, and FtmPT1).

FIG. 24 illustrates the crystal structure of T. acidophilum isopentenyl monophosphate kinase (PDB 3LKK). As shown in panel A, the Poulter lab examined the crystal structure of IPK in complex with IP and ATP to determine which residues (labeled as V73, Y80, V130, and 1140) may be restricting the length of the monophosphorylated substrate. Alanine mutations of these residues improved kinetic properties of IPK with longer prenyl monophosphate substrates. Further engineering could be focused on residues near C₁-C₃ of IP to accommodate branching. Panel B shows a surface depiction of IPK shows a tight hydrophobic pocket with the sp² methyl oriented away from the cleft.

FIG. 25 shows the substrate promiscuity of IPK as determined by low-resolution MS. Compounds in blue (IP, dimethylallyl monophosphate, 4, 8, 1, 5, 9, 2, 6, 3, and 7) were good substrates (approximately >50% conversion) for phosphorylation by IPK. Substrates in red (12, 13, 16, 10, 14, and 17) were poor (approximately <25% conversion) substrates for IPK. Compounds in black (11, 15, geranyl monophosphate, farnesyl monophosphate, and neryl monophosphate) were not substrates for IPK. Reactions were compared against synthetic diphosphate standards.

FIG. 26 illustrates the strategy for kinetic characterization of kinases. As shown in panel A, ADP formation is monitored by a loss in NADH absorbance. In the process, ADP is converted back into ATP, holding the concentration of ATP in the assay constant. As shown in panel B, using isopentenyl monophosphate with IPK as an example, kinases are screened with (red) and without (blue) substrate to determine background rate of ATP hydrolysis. As shown in panel C, a Michaelis-Menten curve is then fitted using a nonlinear regression for the calculation of Kₘ and k_{cat}.

FIG. 27 illustrates the IPK tolerance of alternative substrates. Portions in red highlight rigid portions of substrates. IPK exhibits better kinetic properties with shorter alkyl monophosphates that have flexibility near the monophosphate portion of the molecule. This is reflected in the Kₘ.

FIG. 28 illustrates the biosynthesis of terpenes. As shown in panel A, terpene natural products are composed of hydrocarbon backbones that arise from cyclization of linear prenyl diphosphates. Linear diphosphates are generated by the sequential addition of IPP to DMAPP by prenyltransferases. As shown in panel B, terpenes are functionalized by carbocationic quenching with water (cycloartenol and guaiol), P450 oxidation (betulinic acid), or enzymatic tailoring (paclitaxel). Terpenes can also be combined with other metabolites such as fatty acids (tetrahydrocannabinol, viridicatumtoxin). Bonds and atoms highlighted in red are of IPP and DMAPP origin.

FIG. 29 illustrates hemiterpene analogues. Alcohols were treated with trichloroacetonitrile and phosphorylated with several additions of triethylammonium phosphate solution before being purified on silica.

FIG. 30 illustrates the reported ability of FPPases to use DMAPP analogue. Only analogues with structures that stabilize the allylic carbocation on a tertiary carbon are known substrates for FPPases.

FIG. 31 shows extender unit specificity of wild-type IspA with DMAPP as the starter unit. Conversions were measured by comparing the extracted ion count of the product compared to the amount of starter unit, DMAPP, remaining. For conversion of DMAPP to product with the natural substrate IPP, all extension products (C10 analogues) were summed for the total conversion. The standard deviation of these measurements is 3.2% and was determined by measuring the WT reaction in triplicate.

FIG. 32 shows the active site of E. coli FPPase (PDB 1RQI). Sterically, IspA provides plenty of room for the use of IPP analogues in the active site. As DMAPP is extended and reloaded into the active site for further elongation, the extended diphosphate reaches further into the cavity shown.

FIG. 33 illustrates the extender unit specificity of wild-type IspA with GPP as the starter unit. Conversions were measured by comparing the extracted ion count the product compared to the amount of starter unit, GPP, remaining. No products were detected for 23, 28, and 31. The standard deviation of these measurements is 3.2% and was determined by measuring the WT reaction in triplicate.

FIG. 34 illustrates key NMR assignments and correlations for assignment of the enzymatically generated product GPP-26b. Arrows represent spin coupling observed by ¹H-¹H COSY. Carbon shifts in blue are predicted shifts while those in black were observed.

FIG. 35 illustrates common methods of synthesizing allenes. In mechanism A, an S_{N}2′ method is utilized for allene formation. In mechanism B, addition to a conjugated alkene can provide an allene.

FIGS. 36A-36B illustrate the extension of DMAPP by IspA and mutants. As shown in FIG. 36A, a method was developed to separate the different products of IspA. As showed in FIG. 36B, mutants of IspA found to allow for additional or limit extension events show different product distributions than that of the WT.

FIG. 37 shows the molecular ruler in the IspA active site (PDB 1RQI). Panel A shows where the extender unit nucleophile is in relationship to the starter unit DMAPP. Once extended, the newly formed allylic diphosphate can load back in to the starter unit site for additional extensions. As shown in panel B, in S81F, the phenylalanine mutation prematurely blocks the growing chain cavity limiting IspA to a single extension. As shown in panel C, in the WT, Y80 lies at the bottom of the active site, accommodating the loading of GPP for an extension. As shown in panel D, the Y80D mutation points the active site restricting residue away which enables the WT product, FPP, to load back into the active site for a third extension to generate GGPP.

FIG. 38 illustrates chain length determining mutants and promiscuity towards unnatural extender units with DMAPP as the starter unit. Activities for each enzyme were normalized to the activity of that enzyme with natural substrate, IPP. Errors were within 3.2%.

FIGS. 39A-39B illustrate multiple products formed using alternative substrates. As shown in FIG. 39A, using analogue 18 as an example, after addition of the homoallylic alkene onto C1 of DMAPP, multiple deprotonation events can occur resulting in the formation of various products. As shown in FIG. 39B, when using 18 as a substrate, two different products are observed.

FIG. 40 illustrates terpene diversity stemming from the bisaboyl cation.

FIGS. 41A-41B illustrate methods for measuring terpenes. FIG. 41A shows a comparison of traces from the TIC using EI-MS compared to that of GC-FID shows a cleaner trace when measuring by FID with 10 ng/μL trans-caryophyllene. As shown in FIG. 41B, terpenes that are the same size have very similar linear detection properties when using FID as is observed for trans-caryophyllene and γ-humulene.

FIG. 42 is a plot of aristolochene production over time. Aristolochene is measured using an internal standard of 10 ng/μL trans-caryophyllene.

FIG. 43 shows analogues as substrates for terpene cyclases. Terpene cyclases show promiscuity towards the opposite end of the diphosphate (tail). No examples beyond fluorinated analogues have shown promiscuity at the diphosphate (head) portion of the molecule.

FIG. 44 shows halogenated allylic diphosphates. Minimal studies with halogenated terpene analogues for terpene cyclases have shown an absence of ionization presumably due to the high energy of the resulting carbocation. The use of halogenated DMAPP analogues for prenyltransferase extension suggests that these intermediates are not energetically infeasible.

FIG. 45 illustrates efforts beyond allylic diphosphate substrates. Terpene cyclases proceed through stepwise reaction mechanisms in which allylic diphosphates are ionized to form allylic carbocations. This strict requirement limits diversification of units near the head of the substrate. Engineering of a terpene cyclase capable of cyclizing a non-allylic diphosphate substrate would generate an enzyme that would proceed through an S_{N}2 type reaction that mechanistically would allow for wider substrate tolerance at the head of the diphosphate substrates.

FIG. 46 illustrates the romiscuity of NovQ. Work shows a broad tolerance of NovQ towards various unnatural prenyl donors.

FIG. 47 illustrates the promiscuity of FgaPT2. Work shows a broad tolerance of FgaPT2 towards various unnatural prenyl donors.

FIG. 48 shows in vivo unnatural tryptophan prenylation with cinnamyl alcohol. A. Coupling of the investigated enzymes from previous examples in vivo for the generation of unnatural hemiterpenes and subsequent prenylation of tryptophan. B. The extracted ion chromatogram (EIC) showed a product peak consistent with the in vitro produced analogue

FIG. 49 shows in vivo unnatural tryptophan prenylation with 5-hexyn-1-ol. A. Coupling of the investigated enzymes from previous examples in vivo for the generation of unnatural hemiterpenes and subsequent prenylation of tryptophan. B. The extracted ion chromatogram (EIC) showed a product peak consistent with the in vitro produced analogue.

FIG. 50 shows the hypothetical prenylation of aromatic systems with unnatural prenyl diphosphates from IspA. As ABBA prenyltransferases have shown the ability to use substrates that do not contain allylic diphosphates, these enzymes can presumably accommodate substrates for concerted prenylation events using S_{N}2 mechanisms if sterics allow.

FIG. 51 illustrates the precursor directed diversification of terpenes. Chemical precursors can be transformed into natural product derivatives with non-natural chemical functionality which would enable synthetic diversification.

FIG. 52 illustrates the synthetic biology approach to terpene natural product diversification. Using a synthetic biology approach, a completely unnatural pathway has been assembled that allows for the transformation of chemical precursors into various natural product derivatives. By adding and altering heterologous gene expression, various natural product analogues can be biosynthesized using this platform.

FIG. 53 illustrates the in vivo production of DMAPP from DMAA by PhoN and IPK and subsequent use by FgaPT2. Blue (10, 19, and 23): mass ions were detected by HR-LCMS consistent with the expected product.

FIG. 54 illustrates the reaction catalyzed by PhoN and structures of alcohols tested. Blue (1, 2, 3, 4, 5, 9, 10, 13, 15, 16, 17, 18, 19, 20, 22, and 23): mass ions were detected by HR-LCMS consistent with the expected product.

FIG. 55 is a plot showing the percent yields of PhoN-catalyzed reactions. Error bars are S.D. of the mean (n=2).

FIG. 56 illustrates the reaction catalyzed by FgaPT2 and structures of pyrophosphates tested. Blue (6, 7, 8, 10, 12, 13, 17, 19, 20, 23): mass ions were detected by HR-LCMS consistent with the expected product FIG. 57 is a plot showing the percent conversion of FgaPT2-catalyzed reactions. Error bars are S.D. of the mean (n=2).

FIG. 58 illustrates the reaction catalyzed by FgaPT2 M328G and structures of pyrophosphates tested. Blue (1, 3, 4, 10, 17, 23): products detected by HPLC and confirmed by LR-LCMS with mass ions consistent with the expected product.

FIG. 59 is a plot showing the percent conversion of reactions catalyzed by FgaPT2 wild type or M328G mutant. Error bars are S.D. of the mean (n=2).

FIG. 60 illustrates the reaction catalyzed by CpaD and structures of pyrophosphates tested. Blue (2, 10, 17, 19, 23): products detected by HPLC and confirmed by LR-LCMS with mass ions consistent with the expected product.

FIG. 61 is a plot showing the percent conversion of reactions catalyzed by CpaD with three different cyclic dipeptides.

FIG. 62 illustrates the reaction catalyzed by CpaD I329G and structures of pyrophosphates tested. Blue (1, 3, 4, 10, 17): products detected by HPLC and confirmed by LR-LCMS with mass ions consistent with the expected product.

FIG. 63 is a plot showing the percent conversion of reactions catalyzed by CpaD wild type and I329G mutant with two different cyclic dipeptides.

FIG. 64 illustrates the reaction catalyzed by FtmPT1 and structures of pyrophosphates tested. Blue (2, 10, 17, 19, 23): products detected by HPLC and confirmed by LR-LCMS with mass ions consistent with the expected product.

FIG. 65 is a plot showing the percent conversion of reactions catalyzed by FtmPT1 with three different cyclic dipeptides.

FIG. 66 illustrates the reaction catalyzed by FtmPT1 M364G and structures of pyrophosphates tested. Blue (1, 3, 4, 10, 17): products detected by HPLC and confirmed by LR-LCMS with mass ions consistent with the expected product.

FIG. 67 is a plot showing the percent conversion of reactions catalyzed by FtmPT1 wild type and M364G mutant with two different cyclic dipeptides.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

At various places in the present specification, divalent linking substituents are described. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cu-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, S(O)—CH₃, —CH₂—CH₂—S(O)₂—CH₃, CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)).

Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosone binding site).

The term “culture”, “cultivate”, and “ferment” are used interchangeably and refer to the intentional growth, propagation, proliferation, and/or enablement of metabolism, catabolism, and/or anabolism of one or more cells (e.g., bacteria such as Bacillus cereus). The combination of both growth and propagation may be termed proliferation. Examples include production by an organism of a polyketide of interest. Culture does not refer to the growth or propagation of microorganisms in nature or otherwise without human intervention.

The term “growth” means an increase in cell size, total cellular contents, and/or cell mass or weight of a cell (e.g., bacteria such as Bacillus cereus).

A “growth media” or “growth medium” as used herein can be a solid, powder, or liquid mixture which comprises all or substantially all of the nutrients necessary to support the growth of cells, such as bacterial cells; various nutrient compositions are preferably prepared when particular species are being cultured. Amino acids, carbohydrates, minerals, vitamins and other elements known to those skilled in the art to be necessary for the growth of cells (e.g., bacteria such as Bacillus cereus) are provided in the medium. In one embodiment, the growth medium is liquid. In one embodiment, the growth medium is a production medium (for example, medium optionally containing higher concentrations of glucose and/or altered concentrations of nitrogen).

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

Methods

Provided herein are methods for synthesizing an isoprenoid subunit. In some embodiments, these methods can comprise (i) contacting a primary alcohol defined by Formula I below

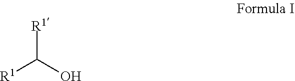

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; with a phosphatase that exhibits bidirectional activity in the presence of ATP to form a phosphate defined by Formula II below

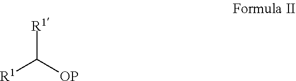

wherein R¹, R^{1′}, and R^{X} are as defined above with respect to Formula I and P represents a phosphate group; and (ii) contacting the phosphate defined by Formula II with a kinase in the presence of ATP to generate the isoprenoid subunit defined by Formula III below

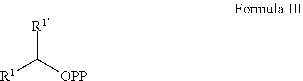

wherein R¹, R^{1′}, and R^{X} are as defined above with respect to Formula I and PP represents a pyrophosphate group.

Phosphatases that exhibit bidirectional activity are known in the art. Such enzymes are known in the art, and classified under Enzyme Commission (EC) numbers 3.1 and 3.2. In some embodiments, the phosphatase can comprise a non-specific acid phosphatase (e.g., an enzyme classified under EC number 3.1.3.2). In certain embodiments, the phosphatase can comprise PhoN. In certain embodiments, the phosphatase can comprise PhoC.

In some embodiments, the kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the kinase can be chosen from a polyphosphate kinase, a phosphomevalonate kinase, a phosphomethylpyrimidine kinase, a farnesyl-diphosphate kinase, or a combination thereof. In certain embodiments, the kinase can comprise isopentenyl phosphate kinase (IPK).

In some cases, the phosphatase, the kinase, or a combination thereof can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

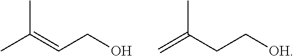

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the phosphatase that exhibits bidirectional activity and the kinase. The cell can be engineered to express (or overexpress) the genes encoding for the phosphatase and the kinase.

Also provided are methods for synthesizing an isoprenoid subunit that comprise (i) providing a cell comprising genes encoding for (1) a phosphatase that exhibits bidirectional activity, and (2) a kinase; and (ii) incubating the cell in a fermentation broth with ATP and a primary alcohol defined by Formula I below

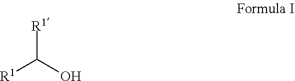

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; thereby generating the isoprenoid subunit.

As described above, phosphatases that exhibit bidirectional activity are known in the art, and classified under Enzyme Commission (EC) numbers 3.1 and 3.2. In some embodiments, the phosphatase can comprise a non-specific acid phosphatase (e.g., an enzyme classified under EC number 3.1.3.2). In certain embodiments, the phosphatase can comprise PhoN. In certain embodiments, the phosphatase can comprise PhoC.

In some embodiments, the kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the kinase can be chosen from a polyphosphate kinase, a phosphomevalonate kinase, a phosphomethylpyrimidine kinase, a farnesyl-diphosphate kinase, or a combination thereof. In certain embodiments, the kinase can comprise isopentenyl phosphate kinase (IPK).

In some cases, the phosphatase, the kinase, or a combination thereof can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

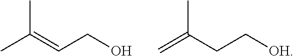

Also provided are methods for synthesizing an isoprenoid subunit that comprise (i) contacting a primary alcohol defined by Formula I below

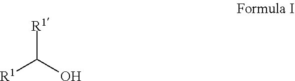

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with the proviso that the primary alcohol defined by Formula I is not one of the following

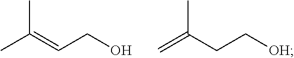

with a first kinase in the presence of ATP to form a phosphate defined by Formula II below

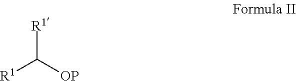

wherein R¹, R^{1′}, and R^{X} are as defined above with respect to Formula I and P represents a phosphate group; and (ii) contacting the phosphate defined by Formula II with a second kinase in the presence of ATP to generate the isoprenoid subunit defined by Formula III below

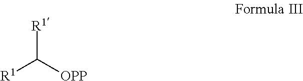

wherein R¹, R^{1′}, and R^{X} are as defined above with respect to Formula I and PP represents a pyrophosphate group.

The kinases can comprise any suitable kinases employ small molecules as acceptors. Such enzymes are classified under EC numbers 2.7.1-2.7.9, and include phosphotransferases with an alcohol group as acceptor, phosphotransferases with a carboxy group as acceptor, phosphotransferases with a nitrogenous group as acceptor, phosphotransferases with a phosphate group as acceptor, phosphotransferases with regeneration of donors, apparently catalyzing intramolecular transfers, diphosphotransferases, nucleotidyltransferases, transferases for other substituted phosphate groups, and phosphotransferases with paired acceptors (dikinases). In some cases, the kinases are kinases that are expressed in soluble form (e.g., in E. coli and/or yeast).

In some embodiments, the first kinase can comprise a kinase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP-thymidine kinases, ADP-thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In certain embodiments, the first kinase, the second kinase, or a combination thereof comprise a mutant enzyme engineered to increase substrate promiscuity, improve enzyme activity, increase enzyme specificity with respect to a particular substrate, or a combination thereof.

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Also provided are methods for synthesizing an isoprenoid subunit that comprise (i) providing a cell comprising genes encoding for a first kinase and a second kinase; (ii) incubating the cell in a fermentation broth with ATP and a primary alcohol defined by Formula I below

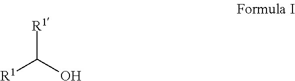

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with the proviso that the primary alcohol defined by Formula I is not one of the following

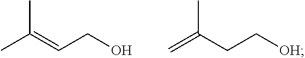

thereby generating the isoprenoid subunit.

The kinases can comprise any suitable kinases employ small molecules as acceptors. Such enzymes are classified under EC numbers 2.7.1-2.7.9, and include phosphotransferases with an alcohol group as acceptor, phosphotransferases with a carboxy group as acceptor, phosphotransferases with a nitrogenous group as acceptor, phosphotransferases with a phosphate group as acceptor, phosphotransferases with regeneration of donors, apparently catalyzing intramolecular transfers, diphosphotransferases, nucleotidyltransferases, transferases for other substituted phosphate groups, and phosphotransferases with paired acceptors (dikinases). In some cases, the kinases are kinases that are expressed in soluble form (e.g., in E. coli and/or yeast).

In some embodiments, the first kinase can comprise a kinase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP-thymidine kinases, ADP-thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In certain embodiments, the first kinase, the second kinase, or a combination thereof comprise a mutant enzyme engineered to increase substrate promiscuity, improve enzyme activity, increase enzyme specificity with respect to a particular substrate, or a combination thereof.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) contacting a primary alcohol defined by Formula I below

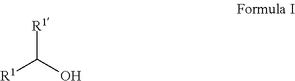

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, with a first kinase in the presence of ATP to form a phosphate defined by Formula II below

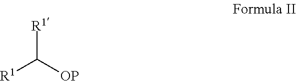

wherein R¹, R^{1′}, and R^{X} are as defined above with respect to Formula I and P represents a phosphate group; and (ii) contacting the phosphate defined by Formula II with a second kinase in the presence of ATP to generate the isoprenoid subunit defined by Formula III below

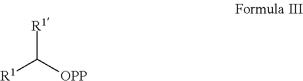

wherein R¹, R^{1′}, and R^{X} are as defined above with respect to Formula I and PP represents a pyrophosphate group; wherein the first kinase, the second kinase, or a combination thereof comprise a mutant enzyme engineered to increase substrate promiscuity, improve enzyme activity, increase enzyme specificity with respect to a particular substrate, or a combination thereof.

The kinases can comprise any suitable kinases employ small molecules as acceptors. Such enzymes are classified under EC numbers 2.7.1-2.7.9, and include phosphotransferases with an alcohol group as acceptor, phosphotransferases with a carboxy group as acceptor, phosphotransferases with a nitrogenous group as acceptor, phosphotransferases with a phosphate group as acceptor, phosphotransferases with regeneration of donors, apparently catalyzing intramolecular transfers, diphosphotransferases, nucleotidyltransferases, transferases for other substituted phosphate groups, and phosphotransferases with paired acceptors (dikinases). In some cases, the kinases are kinases that are expressed in soluble form (e.g., in E. coli and/or yeast).

In some embodiments, the first kinase can comprise a kinase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP-thymidine kinases, ADP-thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In some embodiments, the primary alcohol defined by Formula I is not one of the following

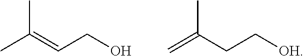

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) providing a cell comprising genes encoding for a first kinase and a second kinase, wherein the first kinase, the second kinase, or a combination thereof comprise a mutant enzyme engineered to increase substrate promiscuity, improve enzyme activity, increase enzyme specificity with respect to a particular substrate, or a combination thereof; (ii) incubating the cell in a fermentation broth with ATP and a primary alcohol defined by Formula I below

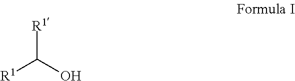

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; thereby generating the isoprenoid subunit.

The kinases can comprise any suitable kinases employ small molecules as acceptors. Such enzymes are classified under EC numbers 2.7.1-2.7.9, and include phosphotransferases with an alcohol group as acceptor, phosphotransferases with a carboxy group as acceptor, phosphotransferases with a nitrogenous group as acceptor, phosphotransferases with a phosphate group as acceptor, phosphotransferases with regeneration of donors, apparently catalysing intramolecular transfers, diphosphotransferases, nucleotidyltransferases, transferases for other substituted phosphate groups, and phosphotransferases with paired acceptors (dikinases). In some cases, the kinases are kinases that are expressed in soluble form (e.g., in E. coli and/or yeast).

In some embodiments, the first kinase can comprise a kinase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP-thymidine kinases, ADP-thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the second kinase can comprise a kinase that uses a phosphate acceptor. Such enzymes are classified under EC numbers 2.7.4, and include phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases. In some embodiments, the second kinase can be chosen from a polyphosphate kinase, a phosphomevalonate kinase, a phosphomethylpyrimidine kinase, a farnesyl-diphosphate kinase, or a combination thereof. In certain embodiments, the second kinase can comprise isopentenyl phosphate kinase (IPK).

In some embodiments, the primary alcohol defined by Formula I is not one of the following

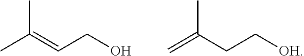

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) contacting a primary alcohol defined by Formula I below

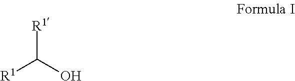

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; with a single enzyme in the presence of ATP to generate the isoprenoid subunit defined by Formula III below

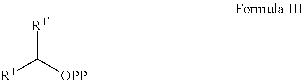

wherein R¹, R^{1′}, and R^{X} are as defined above with respect to Formula I and PP represents a pyrophosphate group, wherein the single enzyme comprises a phosphotransferase that can catalyze both a first phosphorylation and a second phosphorylation of the primary alcohol defined by Formula I to generate the isoprenoid subunit defined by Formula III.

In some embodiments, the single enzyme can comprise a phosphotransferase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP-thymidine kinases, ADP thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the single enzyme can comprise a phosphotransferase that uses a phosphate acceptor. Such enzymes are classified under EC numbers 2.7.4, and include polyphosphate kinases, phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases.

In some embodiments, the single enzyme can comprise isopentenyl phosphate kinase (IPK).

In certain embodiments, the single enzyme can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

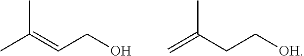

In some embodiment steps (i) and (ii) can be performed in a cell-free system. In some of these embodiments, the method can further comprise recovering the isoprenoid subunit from the cell-free system. In other embodiments, steps (i) and (ii) can be performed in a cell comprising genes encoding for the first kinase and the second kinase. The cell can be engineered to express (or overexpress) the genes encoding for the first kinase and/or the second kinase.

Methods can further comprise introducing the isoprenoid subunit into a natural or artificial isoprenoid biosynthetic pathway to synthesize an isoprenoid. This can be done performed within a cell or in a cell-free system.

Also provided are methods for synthesizing an isoprenoid subunit that comprise (i) incubating a cell in a fermentation broth with ATP and a primary alcohol defined by Formula I below

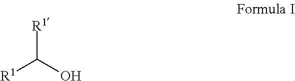

wherein R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, thereby generating the isoprenoid subunit defined by Formula III below

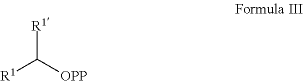

wherein R¹, R^{1′}, and R^{X} are as defined above with respect to Formula I and PP represents a pyrophosphate group; wherein the cell comprises a gene encoding for a phosphotransferase that can catalyze both a first phosphorylation and a second phosphorylation of the primary alcohol defined by Formula I to generate the isoprenoid subunit.

In some embodiments, the single enzyme can comprise a phosphotransferase that uses an alcohol acceptor. Such enzymes are classified under EC numbers 2.7.1, and include hexokinases, glucokinases, ketohexokinases, fructokinases, rhamnulokinases, galactokinases, mannokinases, glucosamine kinases, phosphoglucokinases, 6-phosphofructokinases, gluconokinases, dehydrogluconokinases, sedoheptulokinases, ribokinases, ribulokinases, xylulokinases, phosphoribokinases, phosphoribulokinases, adenosine kinases, thymidine kinases, ribosylnicotinamide kinases, NAD+kinases, dephospho-CoA kinases, adenylyl-sulfate kinases, riboflavin kinases, erythritol kinases, triokinases, glycerone kinases, glycerol kinases, glycerate kinases, choline kinases, pantothenate kinase, pantetheine kinases, pyridoxal kinases, mevalonate kinases, homoserine kinases, pyruvate kinases, glucose-1-phosphate phosphodismutases, riboflavin phosphotransferases, glucuronokinases, galacturonokinases, 2-dehydro-3-deoxygluconokinases, L-arabinokinases, D-ribulokinases, uridine kinases, hydroxymethylpyrimidine kinases, hydroxyethylthiazole kinases, L-fuculokinases, fucokinases, L-xylulokinases, D-arabinokinases, allose kinases, 1-phosphofructokinases, 2-dehydro-3-deoxygalactonokinases, N-acetylglucosamine kinases, N-acylmannosamine kinases, acyl-phosphate-hexose phosphotransferases, phosphoramidate-hexose phosphotransferases, polyphosphate-glucose phosphotransferases, inositol 3-kinases, scyllo-inosamine 4-kinases, undecaprenol kinases, 1-phosphatidylinositol 4-kinases, 1-phosphatidylinositol-4-phosphate 5-kinases, protein-Npi-phosphohistidine-sugar phosphotransferases, shikimate kinases, streptomycin 6-kinases, inosine kinases, deoxycytidine kinases, deoxyadenosine kinases, nucleoside phosphotransferases, polynucleotide 5′-hydroxyl-kinases, diphosphate-glycerol phosphotransferases, diphosphate-serine phosphotransferases, hydroxylysine kinases, ethanolamine kinases, pseudouridine kinases, alkylglycerone kinases, b-glucoside kinases, NADH kinases, streptomycin 3″-kinases, dihydrostreptomycin-6-phosphate 3′a-kinases, thiamine kinases, diphosphate-fructose-6-phosphate 1-phosphotransferases, sphinganine kinases, 5-dehydro-2-deoxygluconokinases, alkylglycerol kinases, acylglycerol kinases, kanamycin kinases, S-methyl-5-thioribose kinases, tagatose kinases, hamamelose kinases, viomycin kinases, 6-phosphofructo-2-kinases, glucose-1,6-bisphosphate synthases, diacylglycerol kinases, dolichol kinases, deoxyguanosine kinases, AMP—thymidine kinases, ADP—thymidine kinases, hygromycin-B kinases, phosphoenolpyruvate-glycerone phosphotransferases, xylitol kinases, inositol-trisphosphate 3-kinases, tetraacyldisaccharide 4′-kinases, inositol-tetrakisphosphate 1-kinases, macrolide 2′-kinases, phosphatidylinositol 3-kinases, ceramide kinases, inositol-tetrakisphosphate 5-kinases, glycerol-3-phosphate-glucose phosphotransferases, diphosphate-purine nucleoside kinases, tagatose-6-phosphate kinases, deoxynucleoside kinases, ADP-dependent phosphofructokinases, ADP-dependent glucokinases, 4-(cytidine 5′-diphospho)-2-C-methyl-D-erythritol kinases, 1-phosphatidylinositol-5-phosphate 4-kinases, 1-phosphatidylinositol-3-phosphate 5-kinases, inositol-polyphosphate multikinases, phosphatidylinositol-4,5-bisphosphate 3-kinases, phosphatidylinositol-4-phosphate 3-kinases, diphosphoinositol-pentakisphosphate kinases, adenosylcobinamide kinases, N-acetylgalactosamine kinases, inositol-pentakisphosphate 2-kinases, inositol-1,3,4-trisphosphate 5/6-kinases, 2′-phosphotransferases, CTP-dependent riboflavin kinases, N-acetylhexosamine 1-kinases, hygromycin B 4-O-kinases, O-phosphoseryl-tRNASec kinases, glycerate 2-kinases, 3-deoxy-D-manno-octulosonic acid kinases, D-glycero-beta-D-manno-heptose-7-phosphate kinases, D-glycero-alpha-D-manno-heptose-7-phosphate kinases, pantoate kinases, anhydro-N-acetylmuramic acid kinases, protein-fructosamine 3-kinases, protein-ribulosamine 3-kinases, nicotinate riboside kinases, diacylglycerol kinases (CTP dependent), maltokinases, UDP-N-acetylglucosamine kinases, and L-threonine kinases. In some embodiments, the first kinase can be chosen from a hexokinase, a glucokinase, a galactokinase, a fructokinase, a glycerol kinase, a choline kinase, a pantetheine kinase, a mevalonate kinase, a pyruvate kinase, an undecaprenol kinase, an ethanolamine kinase, a diacylglycerol kinase, a dolichol kinase, a macrolide 2′-kinase, a ceramide kinase, or a combination thereof.

In some embodiments, the single enzyme can comprise a phosphotransferase that uses a phosphate acceptor. Such enzymes are classified under EC numbers 2.7.4, and include polyphosphate kinases, phosphomevalonate kinases, adenylate kinases, nucleoside-phosphate kinases, nucleoside-diphosphate kinases, phosphomethylpyrimidine kinases, guanylate kinases, dTMP kinases, nucleoside-triphosphate-adenylate kinases, (deoxy)adenylate kinases, T2-induced deoxynucleotide kinases, (deoxy)nucleoside-phosphate kinases, cytidylate kinases, thiamine-diphosphate kinases, thiamine-phosphate kinases, 3-phosphoglyceroyl-phosphate-polyphosphate phosphotransferases, farnesyl-diphosphate kinases, 5-methyldeoxycytidine-5′-phosphate kinases, dolichyl-diphosphate-polyphosphate phosphotransferases, inositol-hexakisphosphate kinases, UMP kinases, ribose 1,5-bisphosphate phosphokinases, diphosphoinositol-pentakisphosphate kinases, (d)CMP kinases, isopentenyl phosphate kinases, (pyruvate, phosphate dikinase)-phosphate phosphotransferases, and (pyruvate, water dikinase)-phosphate phosphotransferases.

In some embodiments, the single enzyme can comprise isopentenyl phosphate kinase (IPK).

In certain embodiments, the single enzyme can comprise a mutant enzyme engineered to increase substrate promiscuity, improve enzyme activity, increase enzyme specificity with respect to a particular substrate, or a combination thereof.

In some embodiments, the primary alcohol defined by Formula I is not one of the following

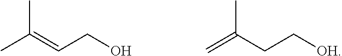

Isoprenoids and Methods of Making Thereof

The methods described above can further comprise introducing the isoprenoid subunit into a natural or artificial isoprenoid biosynthetic pathway to synthesize an isoprenoid. Such biochemical pathways are well known in the art, and described, for example, in the examples below. The isoprenoid subunit can be introduced into a natural or artificial isoprenoid biosynthetic pathway within a cell or in a cell-free system.

As used herein, the term “isoprenoid” refers to a large and diverse class of naturally-occurring class of organic compounds composed of two or more units of hydrocarbons, with each unit consisting of five carbon atoms arranged in a specific pattern. Isoprenoids represent an important class of compounds and include, for example, food and feed supplements, flavor and odor compounds, and anticancer, antimalarial, antifungal, and antibacterial compounds.

As a class of molecules, isoprenoids are classified based on the number of isoprene units comprised in the compound. Monoterpenes comprise ten carbons or two isoprene units, sesquiterpenes comprise 15 carbons or three isoprene units, diterpenes comprise 20 carbons or four isoprene units, sesterterpenes comprise 25 carbons or five isoprene units, and so forth. Steroids (generally comprising about 27 carbons) are the products of cleaved or rearranged isoprenoids.

As used herein, the term “terpenoid” refers to a large and diverse class of organic molecules derived from five-carbon isoprenoid units assembled and modified in a variety of ways and classified in groups based on the number of isoprenoid units used in group members. Hemiterpenoids have one isoprenoid unit. Monoterpenoids have two isoprenoid units. Sesquiterpenoids have three isoprenoid units. Diterpenoids have four isoprene units. Sesterterpenoids have five isoprenoid units. Triterpenoids have six isoprenoid units. Tetraterpenoids have eight isoprenoid units. Polyterpenoids have more than eight isoprenoid units.

Examples of isoprenoids that can be prepared using the isoprenoid subunits described above include the following (as well as derivatives thereof):

Flavors and Fragrances: Myrcene, linalool, limonene, pinene, humulene, caryophellene, menthol, rose oxide, bisabolene, farnesene, farnesol, nootkatone, valencene, cuprene, epi-cubenol, epi-cedrol, a-santalene, vetispiradiene, (+)-curcumene, (+)-turmerone, (+)-dehydrocurcumene, (−)-cubebol, ionone, damascone, 7,11epoxymegastigma 5(6)-en-9-ol, theaspirane, ambrein, and ambrox;

Cannabinoids: tetrahydrocannabinol, cannabidiol, cannabiol, tetrahydrocannabinolic acid, cannabidiolic acid, cannabigerol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran;

Anti-cancer Agents: Bistabercarpamines A and B, β-pinene, 10-O-acetylmacrophyllide, Stylosin, Tabernaelegantine B and D, Perovskiaol, Asperolide A, Clerodane diterpenoid, Caesalppans A-F, Salyunnanins A-F, 7-(2-oxohexyl)-11-hydroxy-6, 12-dioxo-7,9 (11),13-abietatriene, 15-O-β-d-apiofuranosyl-(1→2)-β-d-glucopyranosyl-18O-β-d-glucopyranosyll3(E)-ent-la bda-8 (9),13 (14)-diene3β,15,18-triol, 6E,10E,14Z-(3S)-17-hydroxygeranyllinalool17-O-β-d-glucopyranosyl(1→2)-[α-1-rhamnopyranosyl-(1→6)]β-d-glucopyranoside, Sterebins O, P1, and P2, α-Santalol, Hoaensieremone, Syreiteate A and B, Artemilinin A, isoartemisolide, α-Cadinol, (2R)-pterosin P, Bieremoligularolide, Arbusculin B, α-cyclocostunolide, costunolide, dehydrocostuslactone, Parthenolide, zaluzanin D, and eupatoriopicrin, 1-oxoeudesm-11, (13)eno-12,8α-lactone, Caesalpinone A, Abiesesquine A, Lanosta-7,9, (24)trien-26-oic acid, 1α,2α,8β,9β1,8-bis (acetyloxy)2,9-bis (benzoyloxy)14-hydroxy-β-dihydroagarofuran, Linderolide G, lindestrene, Dihydro-b-agarofuran sesquiterpenes, Dehydrooopodin, Lupeol, 9alisol B, alisol B 23-acetate, Kaunial, 30-hydroxy-11α-methoxy-18β-olean-12-en-3-one′, Asiatic acid, Euscaphic acids G, Hederagenin, Arjunic acid, Schisanlactone C, Schisanlactone D, Schisanlactone H, Kadsulactone, Triregeloic acid, 3-oxo-9-lanosta-7,22Z,24-trien-26,23-olide, 20-hydroxy-24-dammare-n 3-one, bourjotinolone B, (20S,24R) epoxydammarane-12,25diol-3-one, methyl shoreate, Brachyantheraoside A2, 6β-hydroxy-3-oxoolean-12-en-27-oic acid, 3β,6β-dihydroxyolean-12-en-27-oic acid, 3β,24 β dihydroxyolean-12-en-27-oic acid, Urmiensolide B. Urmiensic acid, Neoabiestrine F, Cipaferen H, granatumin E, Neoabieslactone I, taxadiene, Englerin A, cortistatin A, and cyclopamine; and

Anti-Infectious Disease Agents: Artemisinin, Artemisinic Acid, and Ouabagenin

In some embodiments, the isoprenoid can comprise a hemiterpenoid, a monoterpenoid, a sesquiterpenoid, a diterpenoid, a sesterterpenoid, a triterpenoid, a tetraterpenoid, or a higher polyterpenoid. In some aspects, the hemiterpenoid is prenol (i.e., 3-methyl-2-buten-1-ol), isoprenol (i.e., 3-methyl-3-buten-1-ol), 2-methyl-3-buten-2-ol, or isovaleric acid. In some aspects, the monoterpenoid can be, without limitation, geranyl pyrophosphate, eucalyptol, limonene, or pinene. In some aspects, the sesquiterpenoid is farnesyl pyrophosphate, artemisinin, or bisabolol. In some aspects, the diterpenoid can be, without limitation, geranylgeranyl pyrophosphate, retinol, retinal, phytol, taxol, forskolin, or aphidicolin. In some aspects, the triterpenoid can be, without limitation, squalene or lanosterol. The isoprenoid can also be selected from the group consisting of abietadiene, amorphadiene, carene, α-framesene, β-farnesene, farnesol, geraniol, geranylgeraniol, linalool, limonene, myrcene, nerolidol, ocimene, patchoulol, β-pinene, sabinene, γ-terpinene, terpindene and valencene.

In some aspects, the tetraterpenoid is lycopene or carotene (a carotenoid). As used herein, the term “carotenoid” refers to a group of naturally-occurring organic pigments produced in the chloroplasts and chromoplasts of plants, of some other photosynthetic organisms, such as algae, in some types of fungus, and in some bacteria. Carotenoids include the oxygen-containing xanthophylls and the non-oxygen-containing carotenes. In some aspects, the carotenoids are selected from the group consisting of xanthophylls and carotenes. In some aspects, the xanthophyll is lutein or zeaxanthin. In some aspects, the carotenoid is α-carotene, β-carotene, γ-carotene, β-cryptoxanthin or lycopene.

By employing subunits other than dimethylallyl pyrophosphate (DMAPP) and isopentenyl pyrophosphate (IPP), a variety of new isoprenoid structures can be prepared. By way of example, provided are isoprenoids defined by Formula IV below

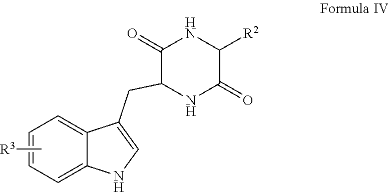

wherein R² is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R³ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R² is hydrogen. In other embodiments, R² can be selected from the group consisting of 6-10 membered aryl, 5-10 membered heteroaryl, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups. In certain embodiments, R² can comprise:

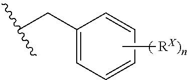

wherein n is 0, 1, or 2 and R^{X} is as defined above with respect to Formula IV.

In some embodiments, R³ can be one of the following:

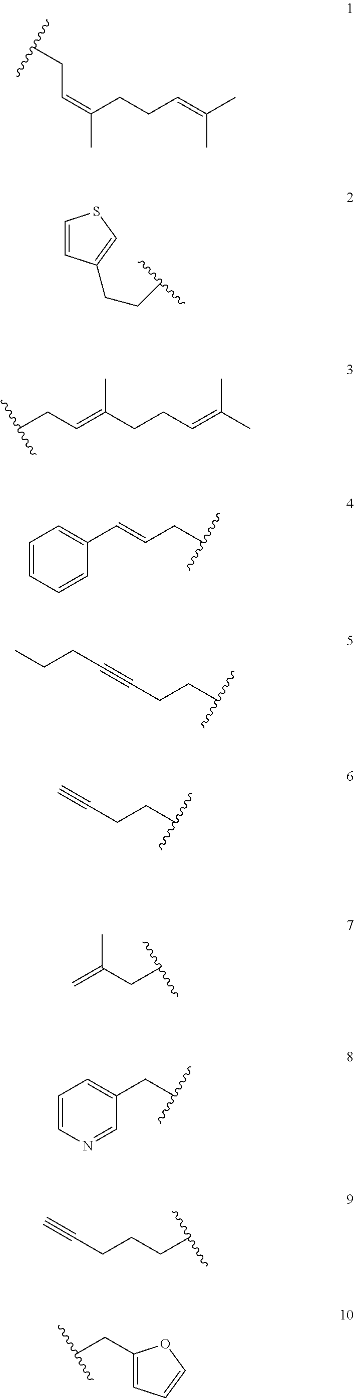

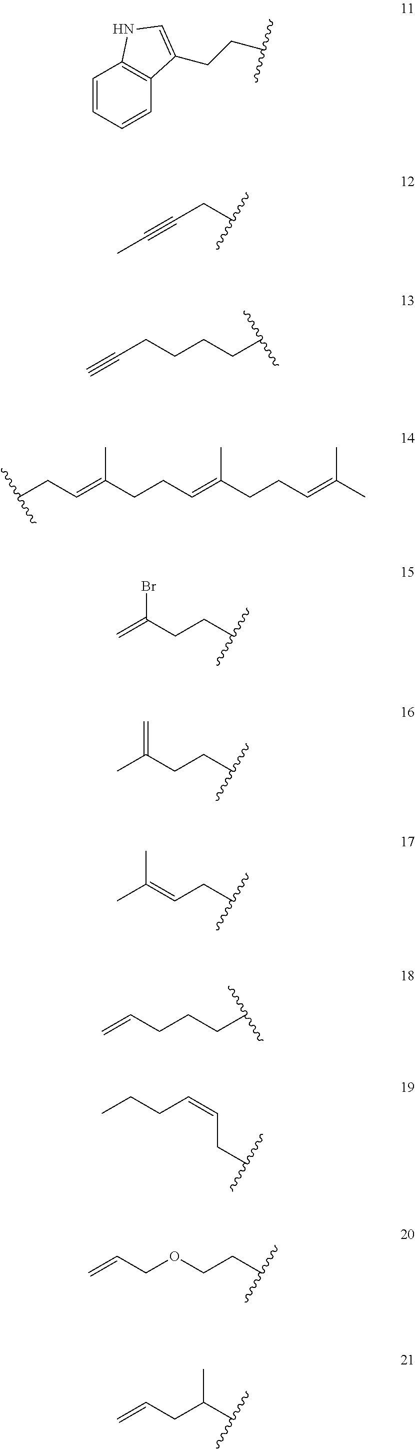

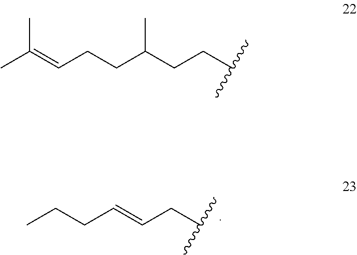

In some embodiments, R³ is not one of the following

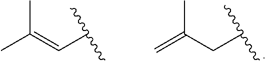

Also provided are isoprenoids defined by Formula V below

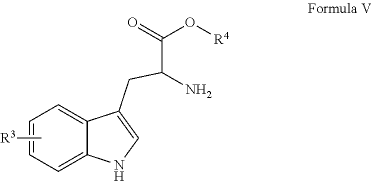

wherein R³ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; R⁴ is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R⁴ can be hydrogen.

In some embodiments, R³ is not one of the following

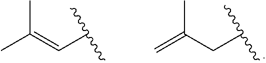

In some embodiments, R³ is one of the following:

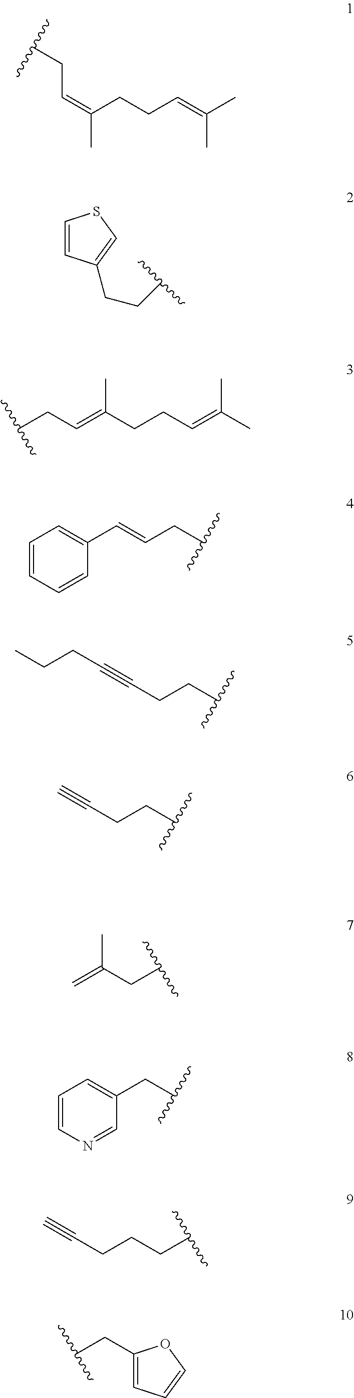

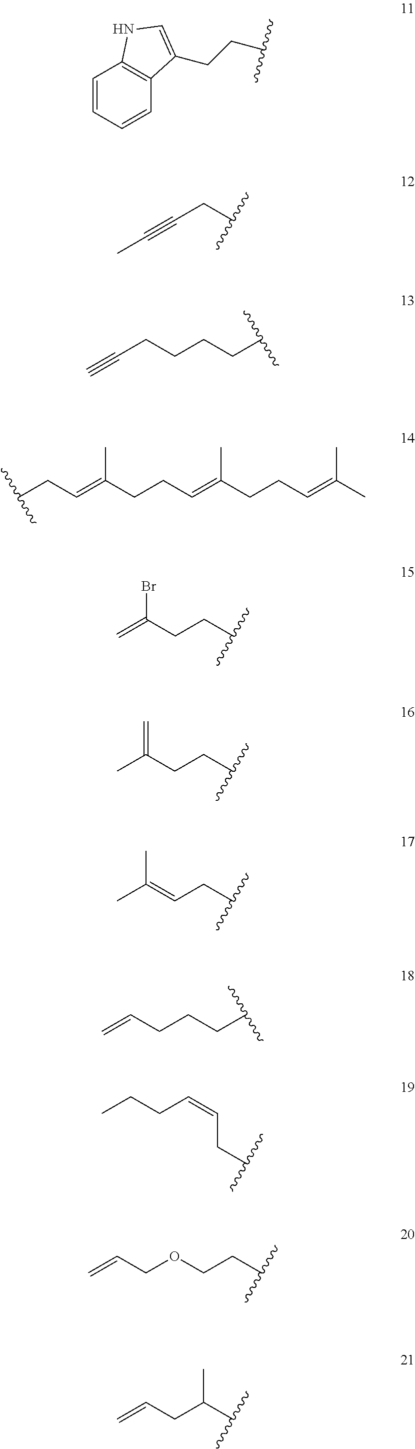

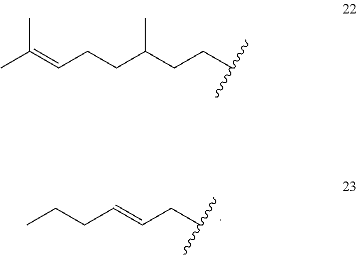

In some embodiments, these isoprenoids can exhibit anticancer activity.

By way of non-limiting illustration, examples of certain embodiments of the present disclosure are given below.

### EXAMPLES

Terpenes are a large class of natural products with wide-ranging biological activities and applications. Previous synthetic biology efforts in this area have focused on producing natural terpenes in microbes either in efforts to increase product titers through pathway engineering or for altering product specificity. However, just two building blocks are used in nature to assemble the carbon scaffolds of terpenes, thus limiting the synthetic scope and utility of natural terpene biosynthetic pathways for the generation of non-natural analogues. In these examples, a comprehensive strategy employing synthetic biology, metabolic engineering, and protein engineering are described that can be used to produce terpenes from non-natural building blocks. This work also provides a platform for the production of terpenes that are site-selectively modified with non-natural chemical functionality, including handles for chemo-selective diversification. Cumulatively, these examples (1) reveal remarkable substrate promiscuity in natural and engineered enzymes, (2) expand the mechanistic understanding of several key enzymes, (3) provide an in vivo and in vitro platform for generation of non-natural terpene building blocks, and (4) provide meroterpene and ergot alkaloid analogues via in vitro and in vivo chemo-enzymatic synthesis.

### Example 1: Introduction to Terpene Biosynthesis and Engineering

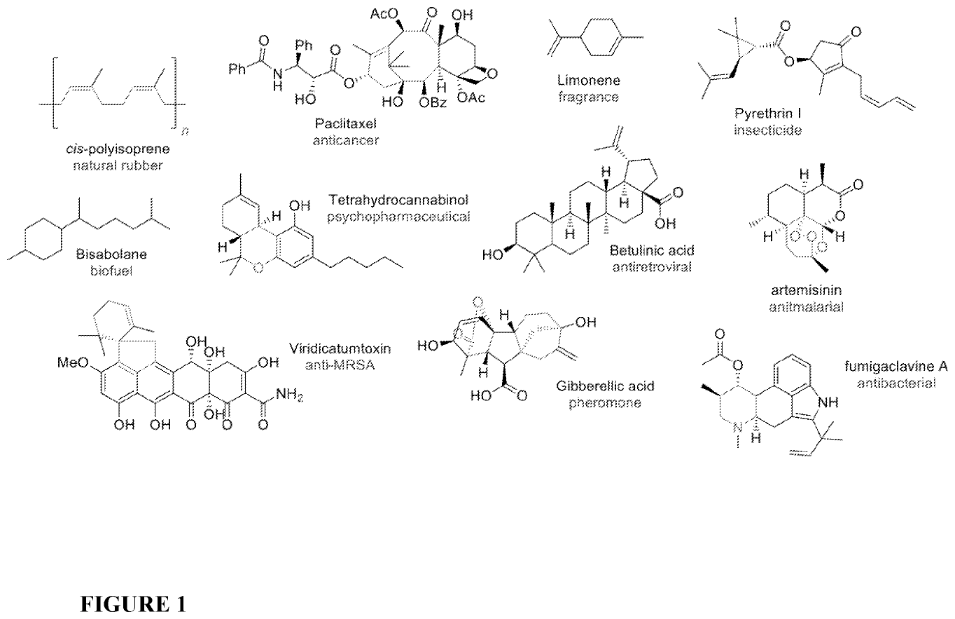

Terpene natural product diversity and biosynthesis. Terpene natural products are used as pharmaceuticals (taxol and artemisinin), pesticides (coumarin and pyrethrin), flavors (hopanoids and menthol), fragrances (citronel and limonene), pigments (carotenoids and xanthophylls), potential biofuels (farnesene) and a variety of other commercial products (FIG. 1). While having applications across a broad range of industries, repurposing of terpenes for pharmaceuticals or agricultural use is stifled due to incomplete structure activity relationships (SAR). This in turn is due to the limited differential reactivity of chemical handles existing in these scaffolds. Accordingly, there is emerging interest in harnessing terpene biosynthetic apparatus to generate non-natural terpene analogues containing varying patterns of oxidation and substitution beyond the naturally available hydrocarbon skeleton. Such prospective diversity elements would open synthetic opportunities for diversification by leveraging the non-native reactivity for chemical derivatization.

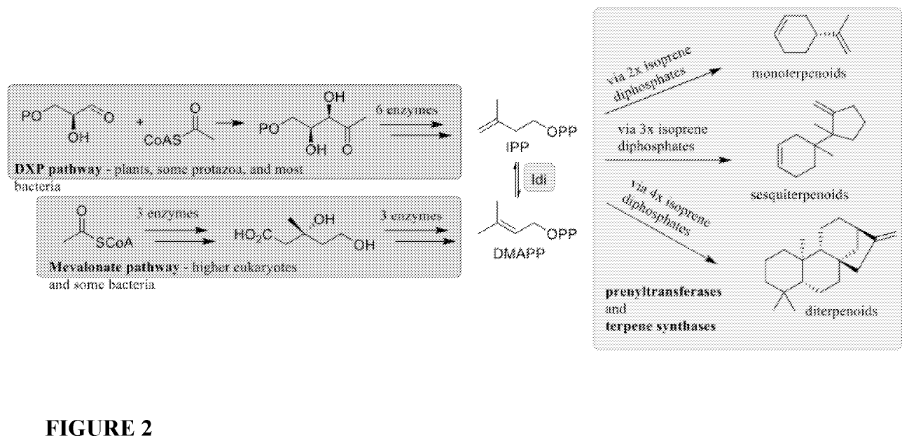

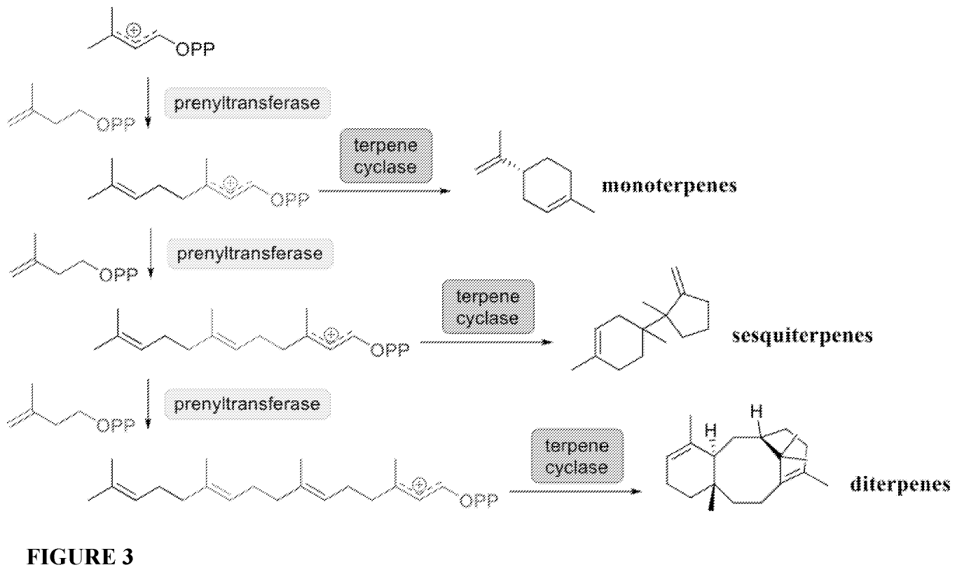

Terpenes are biosynthesized by the successive condensation of the five-carbon (C5-) isoprenes isopentenyl diphosphate (IPP) and dimethylallyl diphosphate (DMAPP) (FIG. 2), collectively referred to as hemiterpenes. In plants, hemiterpenes are either generated by the 1-deoxy-D-xylulose 5-phosphate (DXP) pathway (compartmentalized in plastids) or the mevalonate pathway (cytoplasmic) which allows for various modes of regulation through sequestration, expression, and feedback regulation. Most microbial organisms exclusively use the DXP pathway. Hemiterpenes are stitched together by enzymes classified as prenyltransferases (FIG. 3). Most commonly, DMAPP acts as a starter unit to which a variable number of IPP extender units are added in a process called head-to-tail condensation. Alternatively, head-to-head condensation reactions utilizing two equivalents of DMAPP can occur to generate structural diversity not present in their linear counter-parts. The linear precursors are either transferred to various acceptor molecules or enzymatically cyclized via terpene cyclases and tailored to form a diverse array of carbon and chirality-rich multi-ring compounds.

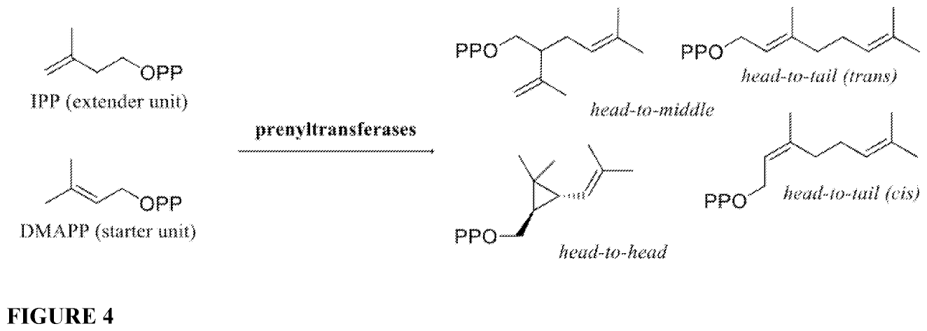

The generation of larger and more complex terpenes occurs through the polymerization of hemiterpenes into longer linear precursors. These precursors are biosynthesized by prenyltransferases that catalyze the elongation of hemiterpenes through the successive addition of IPP onto DMAPP using head-to-tail condensation (FIG. 4). As many as 10 extensions can be catalyzed by a single enzyme. Each reaction proceeds through the generation of an allylic carbocation.

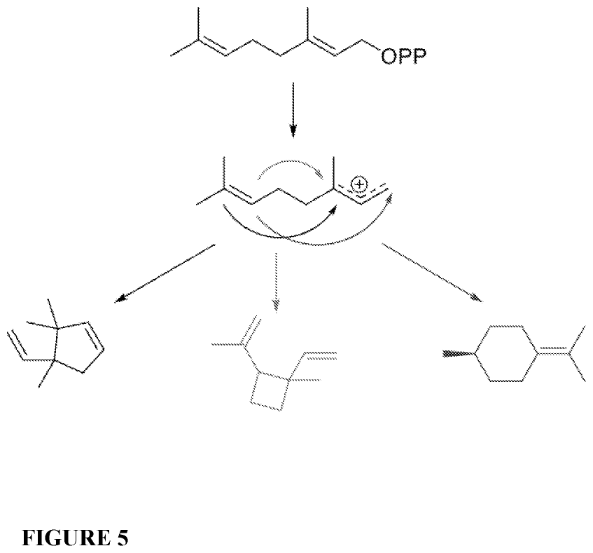

Once the appropriate linear terpene is made, terpene cyclases cleave the allylic diphosphate to generate a highly reactive allylic carbocation (FIG. 5). The electrophilic allylic carbocation undergoes a combination of intramolecular methyl shifts, hydride shifts, and alkene additions. The enzyme dictates the cyclization pattern using a combination of geometry, solvolysis, and electrostatics.

It is this chemical reactivity that is essential to the biosynthesis of more complex terpenes. Enzymes involved in the reaction of these allylic diphosphates must delicately coordinate and facilitate this carbocationic chemistry to afford structurally diverse and mesmerizing compounds from simple achiral five-carbon building blocks.

Heterologous terpene production. Because terpenes are intimately involved in development, growth, reproduction, and signaling, they are tightly regulated and usually produced in limited quantity in non-engineered or native systems. Thus, terpene biosynthetic genes are often transplanted into various heterologous hosts to overcome these limitations. While genetic tools are increasingly becoming available allowing for genome engineering of plants for over production of terpenes and other natural products, generation time, seasonal variations, land use, and processing of plants including extraction and isolation is still quite an extensive effort that is both costly and inefficient.

To increase yields of these valuable products, hosts, often plants, have been engineered by optimizing regulatory factors, increasing the expression of rate-limiting enzymes. Additionally, balancing biochemical precursor flux using strategies such as plant breeding, genetic engineering, and development of scalable plant cell cultures have been explored. Even with these efforts, extraction of these high-value products from natural sources is inefficient, low-yielding, and expensive. For example, a 100-year-old Pacific yew tree can be harvested to produce 300 mg of taxol which, on average, is a sixth of the amount needed to treat a single cancer patient. Plant over-production is hampered by climate and cultivation limitations, product toxicity, and pests. A mere 60 mg/L of 10-deacethylbaccatin III (precursor) can be produced from optimized Taxus baccata cultures after 20 days of fermentation.

Heterologous host systems employing Escherichia coli (E. coli) and Saccharomyces cerevisiae (S. cerevisiae) have greatly simplified efforts towards increasing titers of terpenes due to available genetic resources and cloning tools which enable frameworks that serve as customizable microbial factories. Titers of terpenes have been increased in E. coli and S. cerevisiae by boosting precursor supply and modulating native genes involved in terpene synthesis. For example, while a non-engineered strain of E. coli is only able to generate 10 mg/L of taxadiene (a synthetic precursor to taxol), balancing the expression of genes responsible for the production and consumption of IPP enabled taxadiene titers as high as 10 g/L. Modification of the native DXP pathway in E. coli by a combination of gene deletions and alteration of expression, resulted in a 50-fold increase in carotenoid (a conjugated polyunsaturated terpene) production over the non-engineered system. When the lower-half of the mevalonate pathway from S. cerevisiae was transplanted into E. coli, feeding of mevalonate led to a 5-fold increase in production of carotenoids over the optimized DXP system. While S. cerevisiae has boasted the highest production of IPP, in silico profiling of E. coli and S. cerevisiae using various carbon sources predicts maximum IPP production can be achieved from E. coli using glycerol as a carbon source.

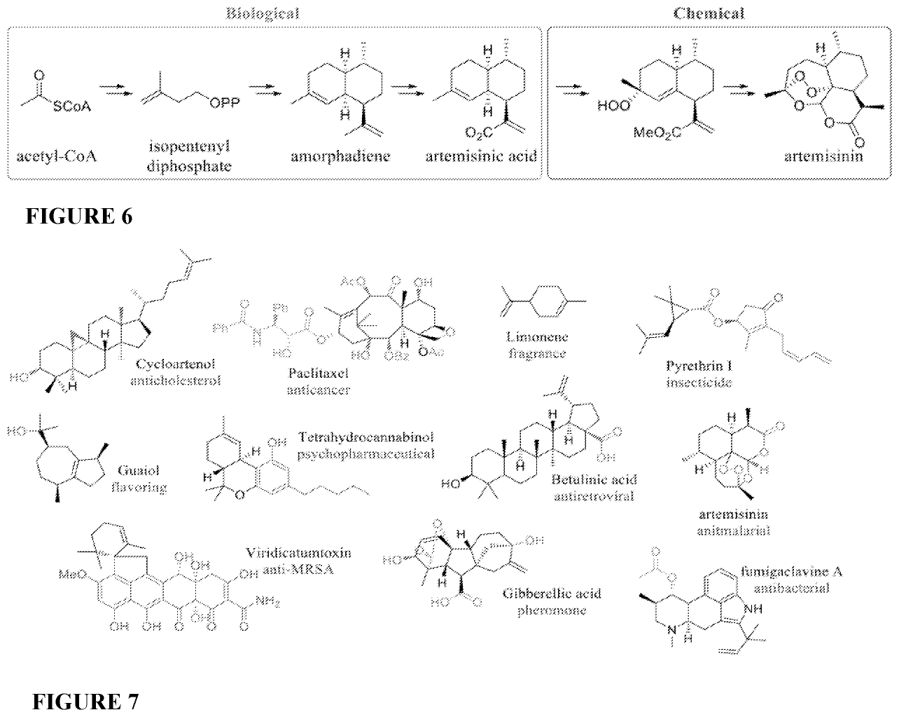

To produce the maximum amount of terpene in a given system, biosynthetic approaches can be blended with more efficient chemical processes. Semi-synthetic routes to terpenes use biosynthesis to generate chemical precursors that through chemical transformations can be converted to their final products (FIG. 6). As P450 identification and activity in heterologous hosts can be difficult, this approach is often taken when P450s prove to be the bottleneck in a fermentation process.

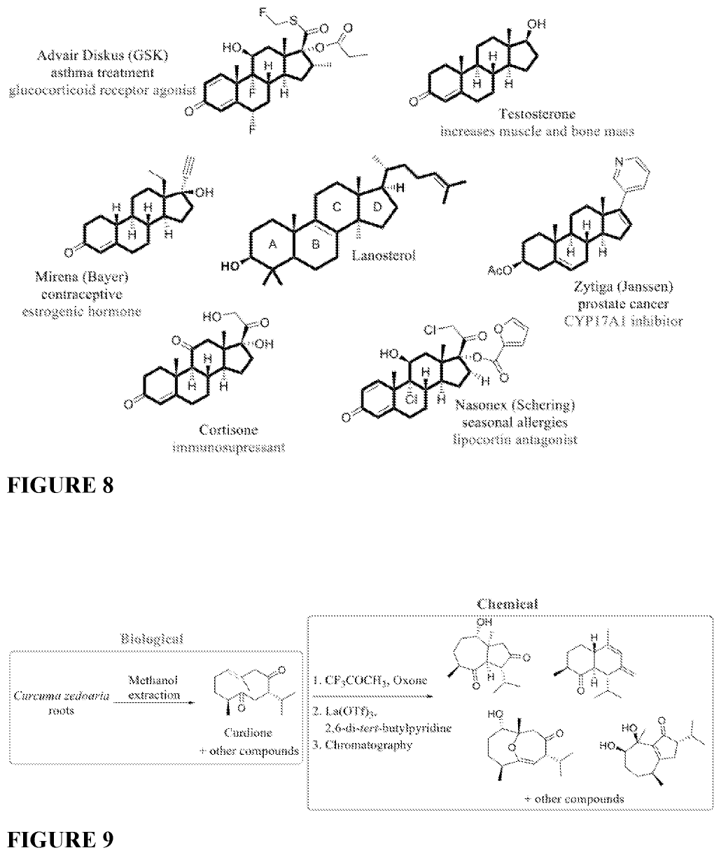

Strategies for terpene natural product diversification. Terpene structures can be diversified through a variety of biosynthetic transformations, most notably P450 oxidation. After oxidation, terpenes can be further modified by acylation, methylation, glycosylation, isomerization, and a variety of other biosynthetic reactions (FIG. 7). The terpene scaffold affords a geometric structure to which points of oxidation afford biological specificity. For instance, the core scaffold of steroids, such as lanosterol, has negligible bioactivity. However once functionalized, by naturally occurring P450s or synthetic diversification, these oxidized scaffolds are highly biologically active and importantly their activities have diverged (FIG. 8).

Terpene natural products can be functionalized by a variety of processes. Usually, the highly saturated and carbon-rich scaffolds of terpenes are not themselves the most facile starting material for chemical diversification as regiospecific oxidation of these highly saturated scaffolds is very challenging. While the tailoring diversity in natural terpenes is extensive, chemists are still limited to modifications on terpene scaffolds that are directed by the usually stringent regio-selectivity of P450s.

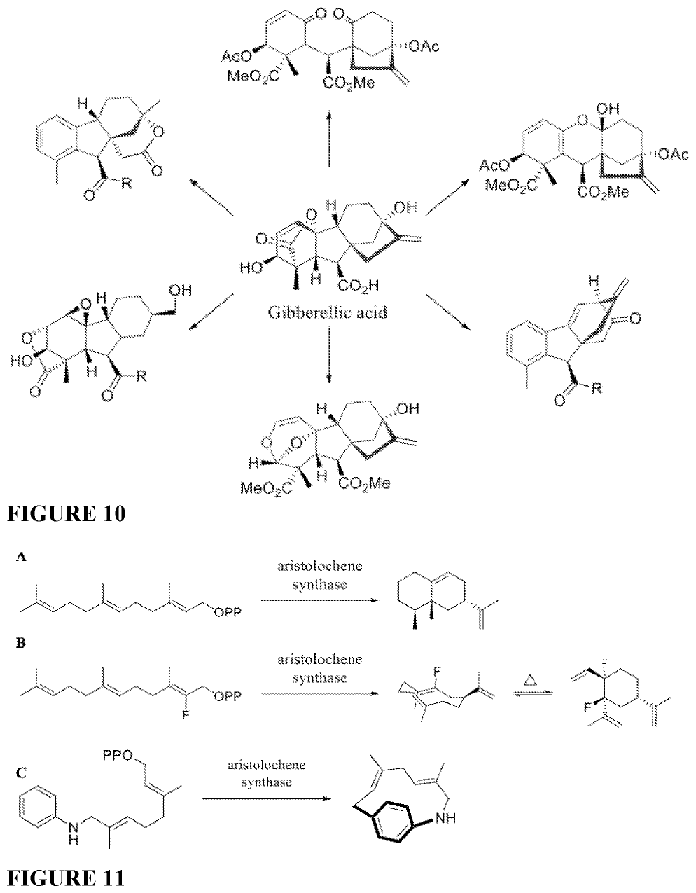

Nevertheless, beyond simple chemical transformations such as acylation of isolated functional groups, chemists have devised a variety of methods that rely on heavily oxidized natural products from nature. Metabolites can be isolated and purified from native hosts or from engineered heterologous hosts by a combination of extraction, chromatography distillation, or recrystallization prior to chemical derivatization however, this can result in an increase costs and waste. Alternatively, methods are being developed for the direct diversification from crude extracts to produce pools of chemically altered natural products that can then be purified by similar methods (FIG. 9). Such strategies must use robust synthetic strategies that target specific chemical moieties already present in natural products. Chemists realizing the difficulty in synthesizing some of the structural moieties contained in terpenes, have also devised methods to unravel these structures into other scaffolds ripe for chemical diversification (FIG. 10).

As diversification of these structures hinges on the modification of existing oxidation and diversity, there has been enormous interest in generating non-naturally occurring oxidation patterns and functionality in terpenes. The most common method of doing so is the mixing of cytochrome P450 monooxygenases with their non-native substrates to capitalize on the substrate promiscuity and product specificity to generate non-naturally oxidized modifications. In these efforts, P450s have also been the target of extensive engineering using a variety of strategies such as DNA shuffling and rational engineering.

One potential route for diversifying the structures of terpenes includes feeding non-natural substrates to terpene biosynthetic pathways in vitro or in vivo. For example, terpene natural products have been inadvertently diversified by using analogues of structural precursors during mechanistic studies. To halt cyclization progression to dissect stepwise mechanisms utilized by terpene cyclases, fluorinated analogues are frequently employed (FIG. 11). Beyond mechanistic studies, initial insight into the promiscuity of these enzymes towards substrate analogues has been leveraged to afford non-natural terpenes.

These methods have been used to study protein prenyltransferases. Farnesyl pyrophosphate analogues containing terminal alkynes for “Click” reactions have been used to study ubiquitination pathways. The corresponding alcohols are synthesized and fed into various cell lines for in vivo phosphorylation by an unknown mechanism.

In order to produce terpene analogues in a sufficient amount for use in chemical libraries or potential drug studies, a different approach to terpene production must be considered. While insightful, the current method of synthesizing chemical precursors for in vitro cyclization is not scalable for adequate production. These pyrophosphorylated analogues are not cell permeable and therefore would be unavailable to whole-cell biocatalysis. In addition, synthesis of these analogues is limited in scope as each analogue needs a dedicated synthetic approach. Ideally a method for the production of analogues could be completed in vivo using cheap chemical precursors shuffled through a flexible pathway.

While there has been minimal insight into the catalytic promiscuity of prenyltransferases and terpene cyclases, the chemistry utilized by these enzymes suggests potential substrate flexibility and malleability for the production of natural product derivatives. A broad range of strategies has been developed to diversify other classes of natural products, but little progress has been made in applying these approaches for the diversification of terpenes, presumably due to the difficulty of applying these approaches in vivo.

Strategies for diversification of polyketides and non-ribosomal peptides. The templated and highly modular logic of polyketide and non-ribosomal peptide synthesis has spurred the development of various strategies that aim to diversify their structures. Precursor directed biosynthesis leverages unnatural building blocks and native biosynthetic machinery to produce unnatural products. An improvement of this strategy, termed mutasynthesis, blocks the availability of the natural substrate so that the unnatural substrate does not compete for incorporation, resulting in a single unnatural biosynthetic product. Potential building blocks can be synthesized using traditional organic synthesis and then used as substrates by biosynthetic machinery in vivo or in vitro for the production of natural product derivatives (FIG. 12). While such derivatives may be available through biological manipulation, feeding of substrates may be more economically feasible as the substrates could be synthesized using robust organic chemistry on a larger scale at a lower initial cost.

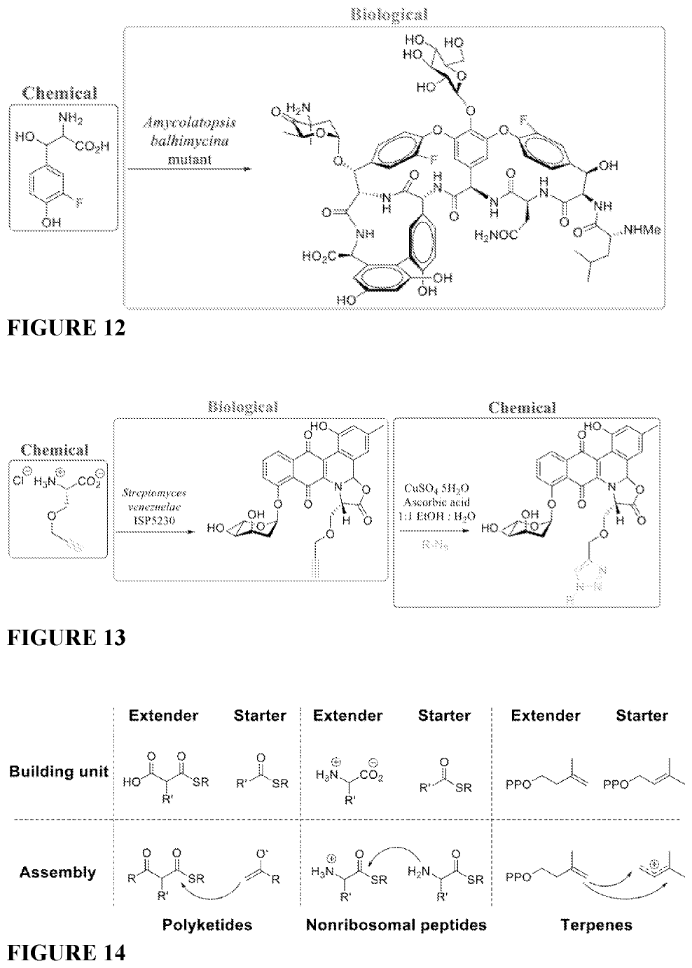

Chemical handles can also be incorporated into natural products via promiscuous or engineered biosynthetic machinery using unnatural building blocks. Such chemical handles may provide sites available for traditional organic synthesis enabling biochemical studies or chemical library construction (FIG. 13). To diversify the Jadomycin antibiotics, the culture media was enriched with unnatural amino acids that were thermodynamically incorporated into the final products, without the use of biological engineering, due to their high concentration. The structure afforded by this method contained an isolated alkyne that provided a bioorthogonal chemical handle for synthetic diversification. Natural building blocks may still be required for viability or for the biosynthesis of the natural product and as such cannot always be removed from the media or organism. Protein engineering enabling broader promiscuity or specificity shift of biosynthetic pathways and development of chemical strategies will expand the utility of mutasynthesis.

While these strategies have been employed for the diversification of non-ribosomal peptides and polyketides, they have yet to be employed to terpene natural products. In systems where these approaches have been implemented, there exists standing chemical diversity already used in the biosynthesis of these natural products (FIG. 1-14). For instance, nonribosomal peptide synthases (NRPSs) use a large variety of amino acid building blocks. Because the identity of the amino acid building blocks utilized by NRPSs does not play a mechanistic role in bond formation, alterations of diversifiable elements have little effect on bond formation, although some other unwanted downstream effects are often observed. In contrast, manipulation of terpene building blocks has been limited due to the extremely narrow diversity contained within two interconvertible building blocks Objective and Scope. The following examples outline and evaluate a precursor directed strategy for terpene diversification that fundamentally alters the composition of terpene scaffolds through the incorporation of non-natural building blocks. In this way, this precursor-based approach could access a completely different and broader chemical space than that of tailoring enzyme-based approaches. The generation of non-natural terpene building blocks can be accomplished using a synthetic biology philosophy whereby chemical precursors can be converted into non-natural building blocks through a completely artificial biosynthetic pathway.

### Example 2. An Artificial Pathway for Isoprenoid Biosynthesis Decoupled from Native Hemiterpene Metabolism

Isoprenoids are constructed in nature using hemiterpene building blocks that are biosynthesized from lengthy enzymatic pathways with little opportunity to deploy precursor-directed biosynthesis. Here, an artificial alcohol-dependent hemiterpene biosynthetic pathway was designed and coupled to several isoprenoid biosynthetic systems, affording lycopene and a prenylated tryptophan in robust yields. This approach affords a potential route to diverse non-natural hemiterpenes and by extension isoprenoids modified with non-natural chemical functionality. Accordingly, the prototype chemo-enzymatic pathway is a critical first step towards the construction of engineered microbial strains for bioconversion of simple scalable building blocks into complex isoprenoid scaffolds.

Introduction

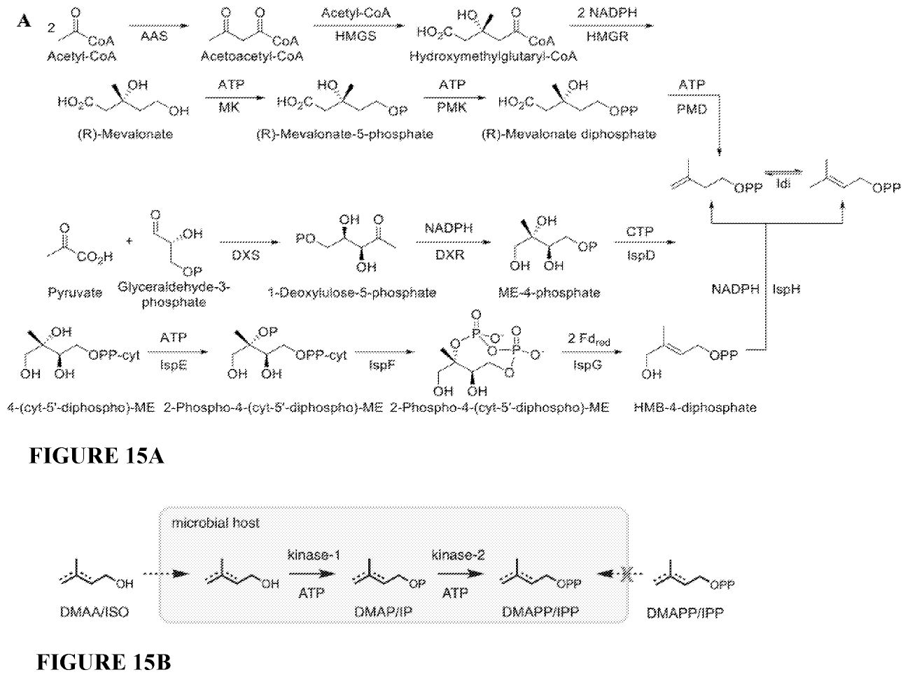

Isoprenoids comprise >55,000 natural products for which methods to access and diversify their structures are in high demand. Ultimately, the isoprene motif plays a critical role in modulating the biological activity of isoprenoids, determines their utility as tools to study and treat human diseases, and provides the basis to develop new fuels and chemicals. Notably, although several valuable isoprenoids have been accessed via heterologous expression, our ability to diversify isoprenoids is extremely limited largely due to critical limitations imposed by native isoprenoid biosynthesis. Firstly, only the mevalonate (MEV) and 1-deoxy-D-xylulose-5-phosphate (DXP) pathways (FIG. 15A) are known to produce the universal hemiterpene isoprenoid diphosphate building blocks, dimethylallyl pyrophosphate (DMAPP) and isopentenyl pyrophosphate (IPP). The negatively charged hemiterpenes are not cell permeable, thus preventing feeding them or analogues thereof into cultures. The MEV and DXP pathways involve at least six enzymatic steps each with stringent substrate specificity and therefore offer little opportunity to diversify the structures of isoprenoids through feeding in non-natural precursors. As a result, while precursor-directed biosynthesis has proven a powerful approach to access diverse structures of natural products-especially polyketides—by feeding non-natural building blocks, this approach has not yet been applied to isoprenoids. Furthermore, late-stage biosynthetic modification of isoprenoid scaffolds is typically limited to oxidations, often catalyzed by P450's. Secondly, terpene metabolism is highly regulated and is a burden to the carbon supply on the cell. For example, the MEV pathway uses three molecules of phosphate donor (ATP) and two reducing equivalents (NADPH) for each DMAPP/IPP, while the DXP pathway requires two phosphate donors (ATP and CTP) and two reducing equivalents (NADPH) (FIG. 15A). Thirdly, given that native terpenes are typically essential for maintenance of the cell, genetic modification of native hemiterpene pathways would likely be lethal.

Together, these limitations could be overcome by supplying a membrane-permeable carbon building block dedicated for a designer pathway that would function independent of native isoprenoid metabolism. A potential strategy for hemiterpene biosynthesis could start with isopentenol (ISO) and dimethylallyl alcohol (DMAA) which are converted to the required diphosphates via stepwise phosphorylation catalyzed by two independent kinases (FIG. 15B). This proposed alcohol-dependent hemiterpene (ADH) pathway is completely orthogonal to the endogenous DMAPP/IPP biosynthetic machinery, such that non-natural precursors are not expected to inhibit endogenous enzymatic machinery. In addition, this route requires only two equivalents of ATP. Furthermore, an artificial pathway designed bottom-up as a replacement for natural hemiterpene biosynthesis could leverage naturally or engineered promiscuous enzymes that enable a broad panel of easily scalable and accessible alcohols to be converted to the corresponding diphosphate, thus providing a strategy to probe the plasticity of downstream isoprenoid biosynthesis in vivo or in vitro.

In this example, as a first step to realizing this goal, the design and development of a prototype ADH pathway that is completely orthogonal to native hemiterpene biosynthesis is described. The ability of this pathway to access isoprenoids is demonstrated by coupling the ADH pathway to two different isoprenoid biosynthetic systems.

Results and Discussion

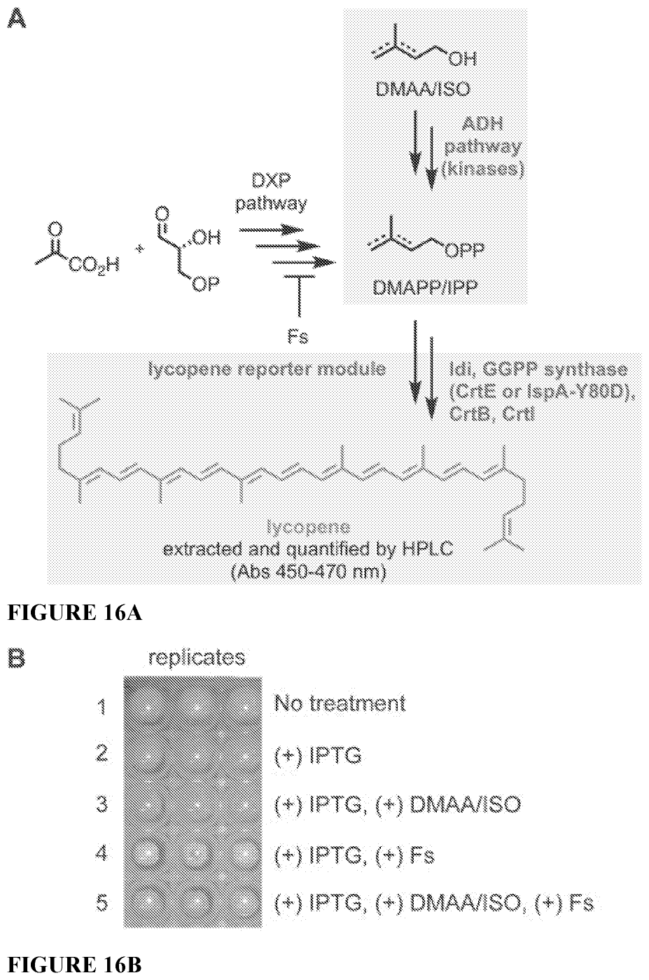

Inspired by the observation that several mammalian cell lines convert farnesol and farnesol analogues to the corresponding diphosphates, it was first determined whether E. coli harbors suitable enzymatic machinery that could convert exogenously provided ISO or DMAA into a pool of hemiterpenes for isoprenoid production. To test this, a reporter system that leverages lycopene biosynthesis was used that includes genes from the CrtEBI operon, a geranylgeranyl diphosphate synthase (CrtE or IspA Y80D), and the isopentenyl diphosphate isomerase ipi (FIG. 16A). In E. coli, this reporter system expresses genes that use pools of hemiterpenes to generate the lycopene pigment, enabling quantification. The intensity of the absorbance at 450-470 nm is directly associated with an increase in hemiterpene production given that the reporter system itself is not rate limiting.

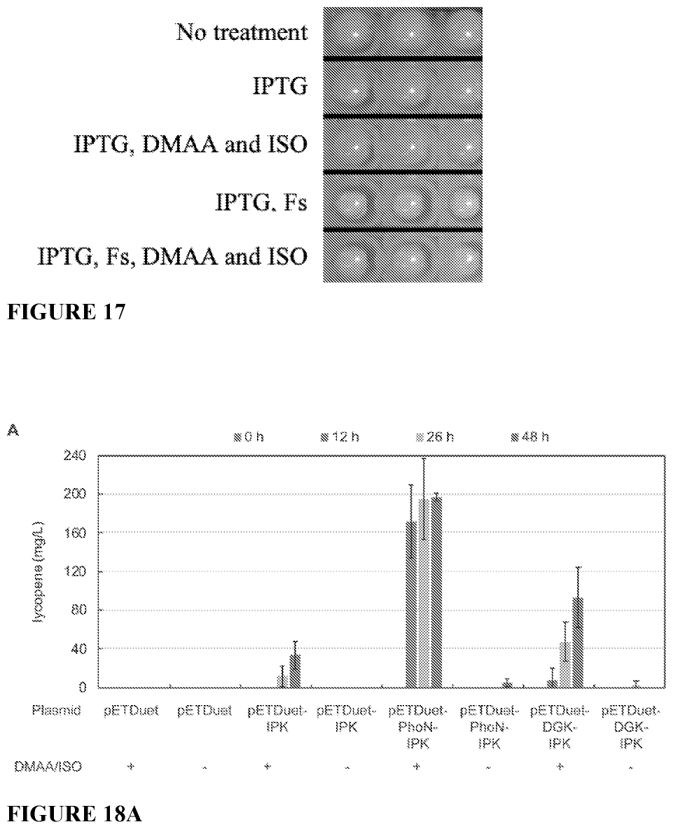

The native DXP pathway in the E. coli reporter strains supports production of lycopene independently of exogenously added DMAA/ISO. Because of this, fosmidomycin (Fs), an inhibitor of the first dedicated step in hemiterpene biosynthesis (FIG. 15A), was leveraged to knock-down endogenous lycopene production in order to determine whether any endogenous machinery could support conversion of DMAA/ISO to hemiterpenes (FIG. 16A). Fs was added to the culture medium at sufficient concentration (0.5 μM) to inhibit the DXP pathway but at low enough concentration growth was not significantly suppressed. In this way, the goal was to employ Fs to prevent accumulation of excess DXP-dependent hemiterpene, forcing production of lycopene solely from the exogenously fed precursors via potential unknown endogenous enzymes. Following addition of DMAA and ISO (each at 2.5 mM) and Fs (0.5 μM) to an overnight culture of E. coli BL21Tuner(DE3) harboring the lycopene reporter plasmids pCDFDuet-GGPP+pACYCDuet-Lyc, lycopene production was quantified by visual examination of the culture broth. However, by comparison of lycopene production to an otherwise identical culture prepared in the absence of DMAA/ISO, E. coli native metabolism was found insufficient to support conversion of ISO and DMAA to lycopene at a rate greater than background conversion via the inhibited DXP-pathway (FIG. 17). Thus, our efforts shifted to identification of enzymes that could be heterologously expressed in E. coli and function as kinase-2 in the proposed ADH pathway.

A protein recently found in archaea, isopentenyl phosphate kinase (IPK), is responsible for the generation of IPP from isopentenyl phosphate (IP) and forms a branch of the MEV pathway called the Archaeal MEV Pathway I (FIG. 15A). Over-expression of IPK in E. coli could lead to improved production of lycopene from exogenously added ISO if (1) an endogenous kinase (kinase-1, FIG. 15B) can convert ISO to the corresponding monophosphate, and (2) the obligatory second phosphorylation (FIG. 15B) contributes to the rate-limiting step in the conversion of ISO to lycopene in E. coli. To test these assumptions, a codon-optimized gene sequence for ipk from Thermoplasma acidophilum (Table 1) was expressed in a strain of E. coli harboring a lycopene reporter module and was incubated either in the presence or absence of supplementary DMAA/ISO, IPTG, and Fs. The expression of IPK clearly resulted in increased lycopene (compare 1-3, FIG. 16B). In addition, the presence of Fs almost completely inhibited lycopene production (2 vs. 4, FIG. 16B). However, an increase in lycopene production was observed when the Fs-treated E. coli cultures that expressed IPK were supplemented with DMAA/ISO (FIG. 16B). This increase in lycopene production that was dependent on IPK and DMAA/ISO validates the assumption that an endogenous kinase is capable of providing IPP from DMAA/ISO and that IPK supports the second required phosphorylation.

TABLE 1

| Protein | Sequence (5′→3′) |
|---|---|
| IPK from | ATGGATCCGTTCACCATGATGATCCTGAAGATTGGCGG |
| Thermoplasma | CAGCGTGATTACCGATAAGAGCGCATATCGCACCGCAC |
| acidophilum codon- | GCACCTACGCCATTCGTAGCATCGTGAAAGTGCTGAGC |
| optimized for | GGCATTGAAGATCTGGTGTGCGTGGTGCATGGCGGTGG |
| E. coli | TAGCTTTGGCCACATCAAGGCGATGGAGTTTGGTCTGC |
| (SEQ. ID 1) | CGGGTCCGAAAAATCCGCGTAGCAGCATCGGCTACAGC |
| | ATCGTGCATCGCGACATGGAAAACCTGGACCTGATGGT |
| | GATCGACGCAATGATCGAGATGGGTATGCGCCCGATTA |
| | GCGTGCCGATTAGCGCCCTGCGTTATGATGGTCGCTTTG |
| | ACTACACCCCGCTGATCCGCTATATTGATGCAGGCTTCG |
| | TGCCGGTGAGCTATGGCGACGTGTATATCAAGGACGAA |
| | CATAGCTATGGCATCTACAGCGGCGACGATATTATGGC |
| | CGATATGGCCGAACTGCTGAAGCCGGATGTGGCCGTGT |
| | TCCTGACCGATGTGGATGGCATCTATAGCAAGGACCCG |
| | AAACGCAATCCGGATGCCGTGCTGCTGCGCGACATCGA |
| | TACAAACATCACCTTCGATCGCGTGCAGAACGATGTGA |
| | CCGGCGGCATTGGCAAGAAATTCGAAAGCATGGTTAAA |
| | ATGAAAAGTAGCGTTAAAAATGGTGTGTACCTGATTAA |
| | TGGCAATCACCCGGAGCGCATTGGTGACATCGGCAAGG |
| | AGAGCTTCATCGGTACCGTGATTCGC |
| DGK from | ATGCCGATGGATCTGCGCGACAACAAACAGAGCCAGA |
| Streptococcus | AGAAATGGAAAAACCGCACCCTGACCAGCAGCCTGGA |
| mutans | ATTTGCCCTGACCGGCATTTTTACCGCCTTCAAAGAAGA |
| codon-optimized | ACGCAACATGAAAAAACACGCCGTGAGCGCACTGCTG |
| for E. coli | GCCGTGATTGCCGGTCTGGTGTTCAAAGTGAGCGTGAT |
| (SEQ. ID 2) | CGAGTGGCTGTTTCTGCTGCTGAGCATCTTCCTGGTGAT |
| | CACCTTCGAGATCGTGAACAGTGCCATCGAGAATGTGG |
| | TGGATCTGGCCAGCGACTATCACTTCAGCATGCTGGCC |
| | AAAAACGCCAAAGATATGGCCGCCGGTGCCGTGCTGGT |
| | TATTAGCGGTTTTGCCGCCCTGACCGGCCTGATTATTTT |
| | TCTGCTGAAAATTTGGTTTCTGCTGTTTCAT |
| PhoN from Shigella | ATGCCGATGGATCTGCGCGACAACAAACAGAGCCAGA |
| flexneri codon- | AGAAATGGAAAAACCGCACCCTGACCAGCAGCCTGGA |
| optimized for | ATTTGCCCTGACCGGCATTTTTACCGCCTTCAAAGAAGA |
| E. coli | ACGCAACATGAAAAAACACGCCGTGAGCGCACTGCTG |
| (SEQ. ID 3) | GCCGTGATTGCCGGTCTGGTGTTCAAAGTGAGCGTGAT |
| | CGAGTGGCTGTTTCTGCTGCTGAGCATCTTCCTGGTGAT |
| | CACCTTCGAGATCGTGAACAGTGCCATCGAGAATGTGG |
| | TGGATCTGGCCAGCGACTATCACTTCAGCATGCTGGCC |
| | AAAAACGCCAAAGATATGGCCGCCGGTGCCGTGCTGGT |
| | TATTAGCGGTTTTGCCGCCCTGACCGGCCTGATTATTTT |
| | TCTGCTGAAAATTTGGTTTCTGCTGTTTCAT |
| FgpaPT2 from | ATGAAAGCCGCAAACGCAAGCAGCGCAGAAGCATATC |
| Aspergillus | GCGTGCTGAGCCGCGCCTTCCGCTTTGACAACGAGGAT |
| fumigatus | CAGAAACTGTGGTGGCACAGCACCGCACCGATGTTTGC |
| codon-optimized | AAAGATGCTGGAAACCGCCAACTATACTACCCCGTGCC |
| for E. coli | AGTACCAGTATCTGATCACCTATAAGGAGTGTGTGATC |
| (SEQ. ID 4) | CCGAGCTTAGGCTGCTATCCTACCAATAGCGCACCTCG |
| | CTGGCTGAGTATCCTGACCCGTTACGGTACCCCGTTTGA |
| | GCTGAGCCTGAACTGCAGCAACAGCATCGTGCGCTACA |
| | CCTTTGAGCCGATTAACCAGCATACCGGCACCGATAAA |
| | GACCCGTTCAACACCCATGCCATTTGGGAAAGTCTGCA |
| | GCATCTGCTGCCGTTAGAGAAGAGCATCGATCTGGAAT |
| | GGTTCCGCCACTTCAAACACGACCTGACCCTGAATAGC |
| | GAAGAAAGCGCCTTTCTGGCCCATAACGATCGCCTGGT |
| | GGGCGGTACCATCCGCACCCAGAACAAACTGGCACTGG |
| | ACCTGAAAGACGGTCGCTTCGCCCTGAAAACCTATATC |
| | TACCCGGCCCTGAAAGCCGTGGTGACCGGCAAGACCAT |
| | TCACGAGCTGGTGTTCGGTAGCGTTCGTCGCTTAGCCGT |
| | TCGCGAACCGCGCATTCTGCCGCCGCTGAACATGCTGG |
| | AAGAGTATATCCGTAGCCGCGGCAGCAAAAGCACCGC |
| | AAGTCCGCGTCTGGTTAGCTGTGACCTGACCAGCCCGG |
| | CAAAAAGCCGCATCAAAATCTACCTGCTGGAGCAGATG |
| | GTGAGCCTGGAAGCCATGGAAGATCTGTGGACATTAGG |
| | CGGCCGTCGCCGTGATGCCAGCACCCTGGAAGGTCTGA |
| | GCCTGGTTCGTGAACTGTGGGATCTGATTCAGCTGAGC |
| | CCGGGCCTGAAAAGCTACCCTGCCCCGTATCTGCCGCT |
| | GGGTGTGATTCCTGACGAGCGTTTACCGCTGATGGCCA |
| | ATTTTACCCTGCACCAGAACGATCCGGTGCCGGAACCG |
| | CAGGTGTACTTTACAACCTTCGGCATGAATGACATGGC |
| | CGTGGCCGATGCACTGACCACCTTTTTTGAGCGTCGCG |
| | GCTGGAGTGAAATGGCACGCACCTATGAAACCACCCTG |
| | AAGAGCTACTACCCGCATGCCGACCATGATAAACTGAA |
| | CTATTTACATGCCTACATCAGCTTTAGCTATCGCGATCG |
| | CACCCCGTATCTGAGTGTGTACCTGCAGAGCTTCGAAA |
| | CAGGTGACTGGGCCGTGGCAAACCTGAGCGAAAGCAA |
| | GGTGAAATGCCAGGATGCCGCCTGTCAGCCGACAGCAC |
| | TGCCGCCTGATCTGAGTAAAACCGGCGTGTACTACAGC |
| | GGCCTGCAT |

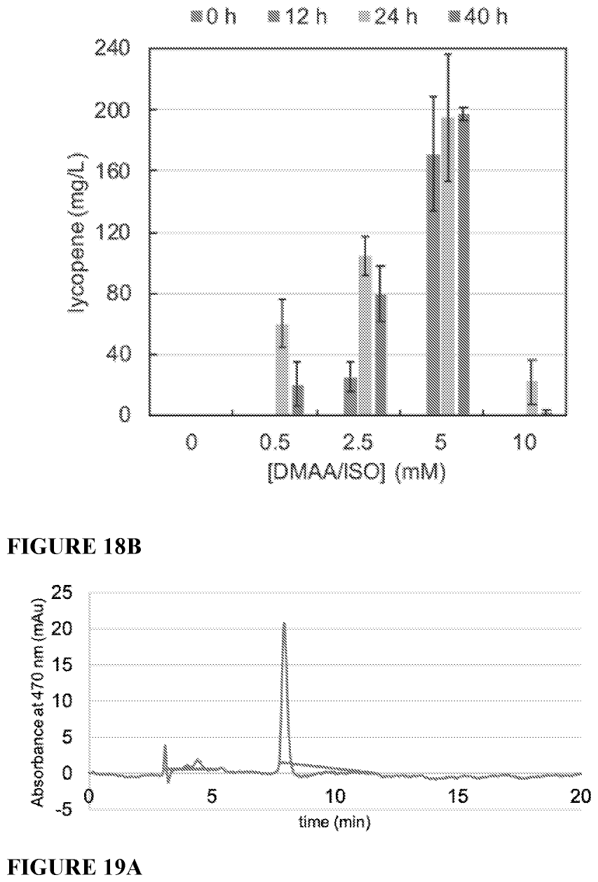

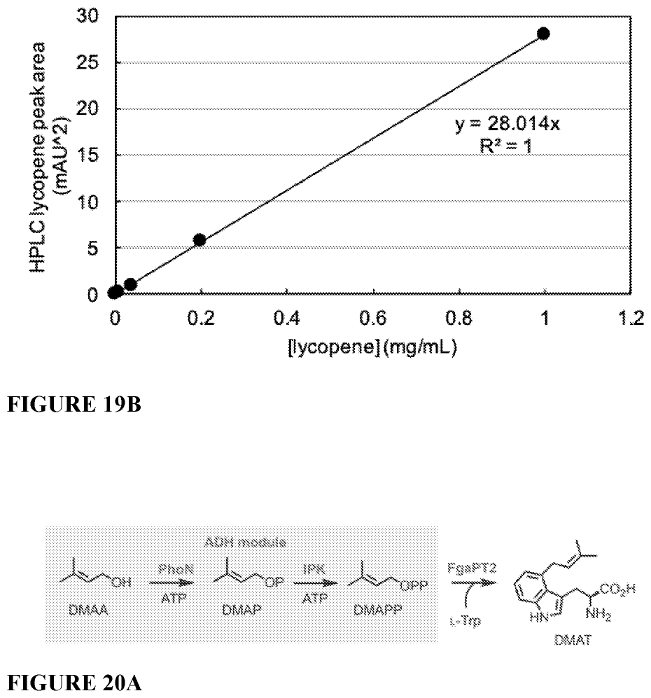

It was reasoned that over-expression of the E. coli gene product putatively responsible for DMAA/ISO phosphorylation would result in improved production of hemiterpenes. In a preliminary attempt to identify a suitable enzyme that could act as ‘kinase-1’ (FIG. 15B), a set of 12 soluble alcohol kinases from E. coli, S. cerevisiae and A. thaliana, in addition to PhoN, a class-A non-specific acid phosphatase from Shigella flexneri (Table 2) were cloned, expressed in E. coli, subjected to immobilized-metal affinity chromatography, and analyzed for their ability to phosphorylate DMAA/ISO by LC-MS analysis. PhoN was included in this set as it phosphorylates various alcohols in vitro. Notably, while none of the kinases displayed the desired activity, mass ions consistent with DMAPP (calculated 165.0317 m/z, [M−H]⁻; observed 165.0318 m/z, [M−H]⁻) and IPP (calculated 165.0317 m/z, [M−H]⁻; observed 165.0317 m/z, [M−H]⁻) were detected in the presence of PhoN. Another potential candidate for kinase-1 is a membrane-associated diacylglycerol kinase (DGK) from Streptococcus mutans which is known to display undecaprenol-kinase activity. Given that DGK is membrane-bound, it was tested in vivo. In parallel, PhoN was also tested in vivo to ensure that its activity could contribute to isoprenoid biosynthesis. Accordingly, PhoN and DGK (Table 1) were each cloned into pETDuet-IPK and tested for their ability to support isoprenoid production in an E. coli lycopene strain by extraction and HPLC-based quantification of the pigment (FIG. 18A and FIGS. 19A-19B). Remarkably, the prototype PhoN-IPK system was capable of converting DMAA/ISO to lycopene in titers of ˜150 mg/L in E. coli after 12 hours post-induction (FIG. 18A). These titers are comparable to that of an optimized engineered DXP pathway (24 mg/L) and heterologous production of the MEV pathway (102 mg/L) in E. coli. As expected, the activity was largely dependent on the presence of PhoN given that 17-fold less lycopene was produced at 26 hours post-induction when PhoN is absent. In addition, wild-type DGK could support lycopene production in good yields, although these were 4-fold lower than that with PhoN after 24 hours.

TABLE 2
Summary of wild-type activities of kinases tested for phosphorylation of ISO and DMAA.
Data was retrieved using the BRENDA database.

| Enzyme (BRENDA Entry) | Reaction catalyzed |
|---|---|
| S. cerevisiae Glycerol kinase (EC2.7.1.30) | 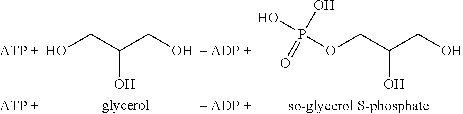 |
| S. cerevisiae Homoserine kinase (EC2.7.1.39) | 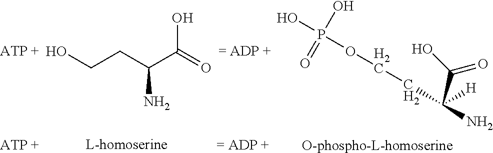 |
| E. coli Phosphoribosylpyro- phosphate synthetase (EC2.7.6.1) | 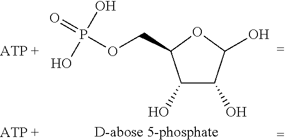 |
| | 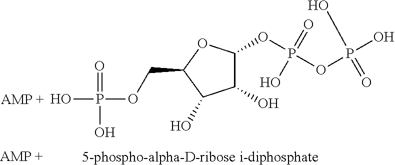 |
| E. coli Homoserine kinase (EC2.7.1.39) | 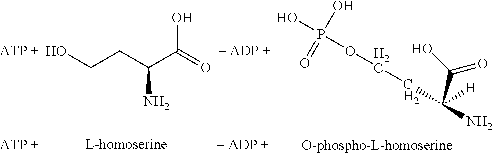 |
| E. coli Ethanolamine kinase (EC2.7.1.82) | 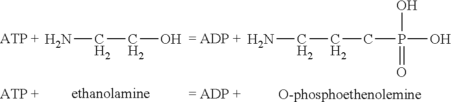 |
| E. coli Hydroxyethylthiazole kinase (EC2.7.1.50) | 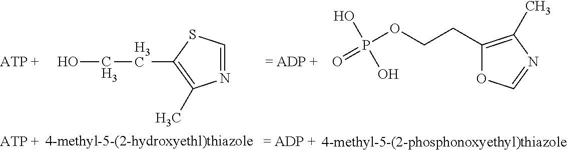 |
| E. coli Undecaprenol kinase (EC2.7.1.66) | 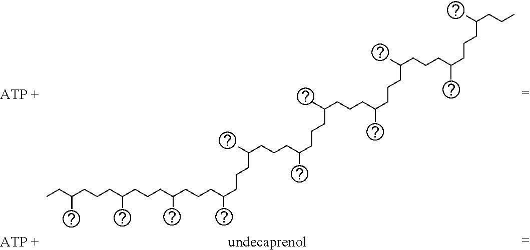 |
| | 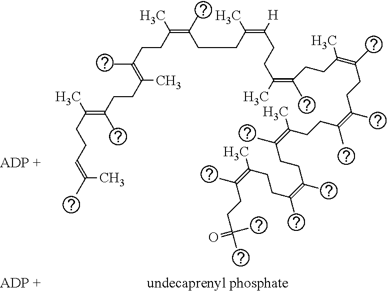 |
| E. coli Diacylglycerol kinase (EC2.7.1.107) | 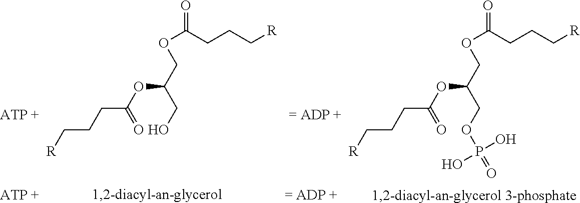 |
| E. coli Glycerol kinase (EC2.7.1.30) | 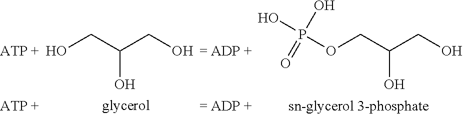 |
| E. coli 4-diphosphocytidyl- 2-C-methyl-D-erythritol kinase (IspE) (EC2.7.1.148) | 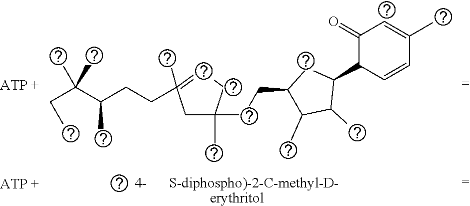 |
| | 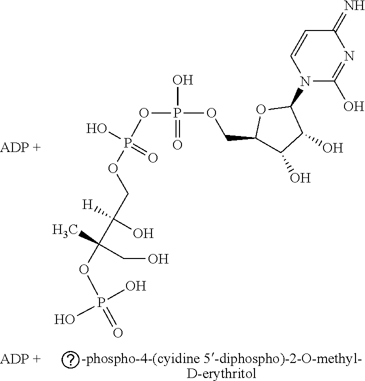 |
| Arabidopsis thaliana Farnesol kinase (EC2.7.1.216) | 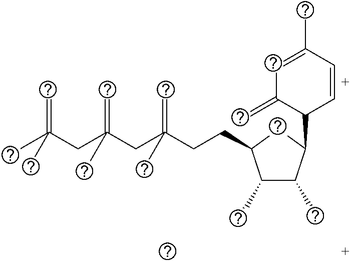 |
| | 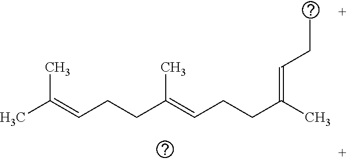 [SMILES: CC/C=C(\C)CC/C=C(\C)CCC=C(C)C] |
| | 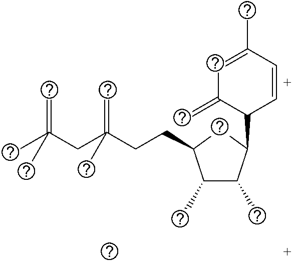 |
| | 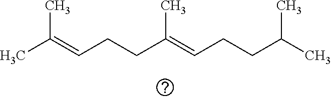 |
| Thermoplasma acidophilum Isopentenyl phosphate kinase (IPK) (EC2.7.4.26) | 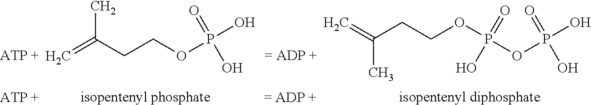 |
| Shigella flexneri Non- specific acid phosphatase (PhoN) (EC3.1.3.2) | 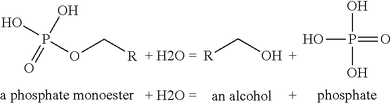 |
| Streptococcus mutans Diacylglycerol kinase (DGK) (EC2.7.1.107) | 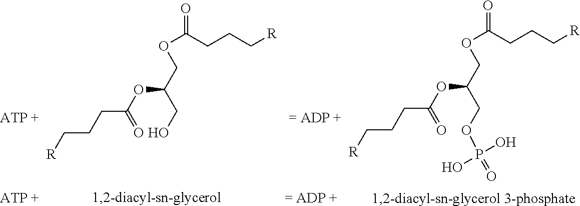 |

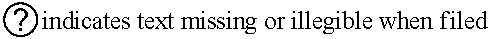

Next, to determine whether the lycopene titers were dependent on the concentration of the exogenously provided alcohol substrates, a series of lycopene assays were carried out at various concentrations of DMAA/ISO using the strain harboring pETDuet-PhoN-IPK/pAC-LYCipi in the presence of Fs (FIG. 18B). After 24 hours, the prototype strain produced ˜60 mg/L lycopene at 0.5 mM each of DMAA/ISO, ˜100 mg/L at 2.5 mM each of DMAA/ISO, and ˜190 mg/L at 5 mM each of DMAA/ISO (FIG. 18B). At concentrations of 10 mM, DMAA/ISO supported a maximum lycopene titer of ˜20 mg/L after 24 hours.

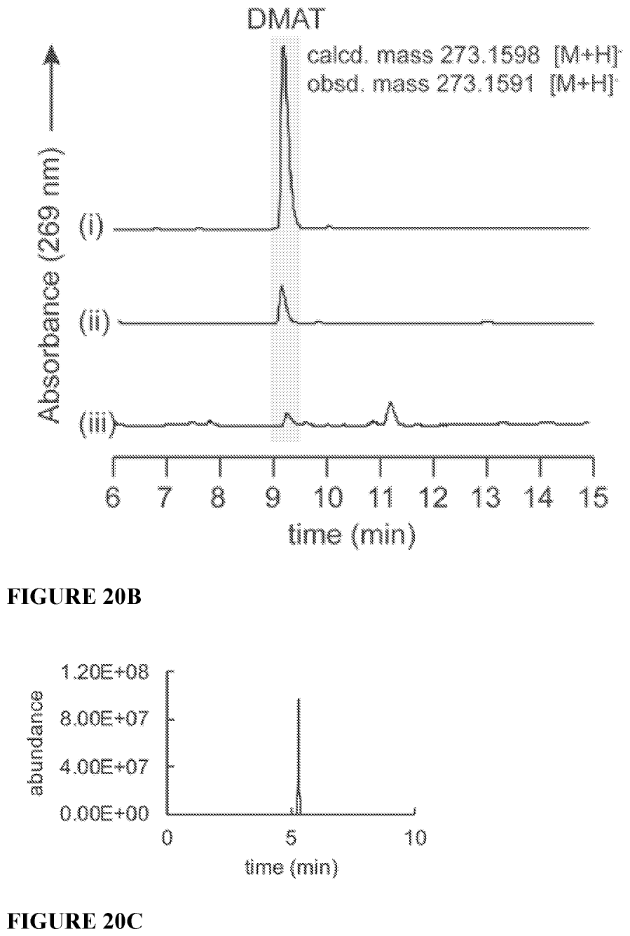

As an additional test of the ability of the designed prototype strain to provide an isoprenoid building block, the PhoN-IPK pathway was used to provide DMAPP which was then transferred to L-Trp using the prenyltransferase (PTase) FgaPT2 by providing an additional plasmid that expressed the PTase (FIG. 20A). First, an in vitro reaction using purified FgaPT2 and a DMAPP standard confirmed production of a single product by HPLC and LC-MS analysis of the product mixture (FIG. 20B), the mass of which (273.1591 m/z, [M+H]⁺) was consistent with the expected regioselectively mono-alkylated product, dimethylallyl-L-Trp (DMAT, calculated 273.1598 m/z, [M+H]+). In addition, using purified PhoN, IPK, and FgpaPT2 in a one-pot reaction, the same DMAT product was detected when DMAA was included in the in vitro reaction mixture (FIG. 20B), confirming the ability of the ADH pathway to produce the required prenyl donor for FgaPT2. To test the ability of the in vivo system to provide the hemiterpene and couple it with the PTase, DMAA/ISO were fed into E. coli that harbored the ADH module (pETDuet-PhoN-IPK) in conjunction with a plasmid that harbored the PTase (pCDFDuet-FgaPT2), and after protein expression, HPLC confirmed the presence of the expected product (DMAT) in the culture media with an elution time indistinguishable from that produced by both in vitro reactions (FIG. 20B). As expected from the in vitro reactions, an extracted-ion chromatogram of the culture media revealed a single peak corresponding to the mass ion for DMAT (FIG. 20C). By comparison of the DMAT peak area of the in vitro reaction with DMAPP (which went to completion) to that of the in vivo reaction, the ADH-FgaPT2 pathway was judged to support the production of DMAT at ˜20 mg/L. Furthermore, DMAT was not detected by HPLC in negative controls that lacked either the alcohol or FgaPT2 enzyme and was 100-fold lower in abundance in the negative controls, as detected by HR-MS analysis (data not shown), confirming that the artificial ADH module is required for high-level production of the prenylated product

Conclusion

In summary, an artificial hemiterpene biosynthetic pathway dependent on the exogenous addition of DMAA/ISO was developed. The prototype ADH pathway performed similarly to previously established routes that depend on building blocks from primary metabolism. It is expected that ribosome binding site and/or promoter engineering can be leveraged to optimize the productivity of the pathway further. Notably, in contrast to synthetic biology efforts that have for example constructed a new entry point into the DXP pathway and provided new routes to DXP, the ADH pathway described here is the first to transform scalable simple precursors directly into the required pyrophosphates and couple them to isoprenoid biosynthesis. This provides a simple strategy to provide isoprenoids in good yields given that only two enzymes and DMAA/ISO need to be provided. Indeed, in the absence of ISO/DMAA, there was insufficient endogenous DMAPP in E. coli to support high level production of the prenylated tryptophan, even when Fs was not used to inhibit the native DXP pathway. These methods can be used as a future discovery tool that enables the in vivo biosynthesis of hemiterpene analogues, and by extension, non-natural isoprenoids.

For example, PhoN displays a broad specificity in vitro, and this is expected to extend to the in vivo system here. Furthermore, several features of PhoN have been targeted by enzyme engineering, including shifting its pH optima for neutral media and improving its kinase activity with concomitant reduction in phosphatase activity. Similarly, although the promiscuity of IPK is largely under-explored, its substrate interacts with the enzyme active site through electrostatic forces dictated by the phosphate portion of the substrate, while the remaining alkyl portion of the substrate is simply sterically accommodated. Indeed, the substrate specificity of IPK has been expanded to include geranyl- and farnesylphosphate. An expanded set of non-natural hemiterpenes provided by the prototype or engineered ADH pathway could be coupled with downstream enzymes to probe the promiscuity and utility of isoprenoid biosynthesis. For example, it is expected that this precursor-directed approach to non-natural isoprenoids can be readily extendible to natural product scaffolds that include L-Trp and/or other aromatics given the reported promiscuity of aromatic PTases. Subsequently, the ADH pathway may enable the production of prenylated and terpene natural products with non-natural alkyl groups expanding upon the limited chemical diversity afforded by nature.

Materials and Methods

General. All plasmids were verified by DNA sequencing. Purifications of all DNA were performed with kits from BioBasic. Lycopene standard was purchased from Sigma Aldrich. Synthetic oligonucleotides were purchased from IDT (Coralville, Iowa, USA). Plasmid pAC-LYCipi was purchased from Addgene (Plasmid #53279, Addgene, Cambridge, Mass., USA). Restriction enzymes were purchased from New England Biolabs (Ipswich, Mass., USA). Polymerase chain reactions were conducted using Phire Hot Start II DNA Polymerase from ThermoFisher Scientific (Waltham, Mass., USA).

Cloning Candidate Kinase Genes. Genes were amplified from E. coli BL21 and S. cerevisiae EBY100 genomic DNA by taking 100 μL of cell pellet, adding 200 μL of water, followed by boiling in a 1.5 mL tube for 15 min. The cell debris was pelleted and 1 μL of the supernatant was used for PCR from genomic DNA using the primers listed in Table 3. Farnesol kinase from A. thaliana was PCR amplified from a cDNA library gifted from the lab of Dr. José M. Alonso (NC State, Department of Genetics) using the primers listed in Table 3. The PCR reaction contained 5× Phire II buffer, 0.2 mM dNTPs, 0.5 μM each primer, 1 μL Phire II DNA polymerase, and 1 μL of genomic DNA, in a total volume of 50 μL. The cycling parameters used were as follows: 1) 98° C., 30 s; 2) 98° C., 5 s; 3) 66° C., 15 s; 4) 72° C., 20 s; 5) repeat steps 2-4 34 times; 6) 72° C., 1 min; 7) 4° C., hold. Following amplification, the amplified products were gel purified, digested with BamHI and NotI, and ligated into similarly treated ‘empty’ pETDuet and pETDuet-IPK (in MCS2). Ligation mixtures were transformed into chemically competent E. coli NovaBlue (DE3) cells (Novagen) and plated on LB agar supplemented with 50 μg/mL kanamycin for incubation overnight at 37° C. Colonies were then screened for the appropriate size insert by colony PCR using primers annealing to the T7 promoter and T7 terminator. Those colonies with correct sized inserts were then picked and grown in 3 mL LB supplemented with 50 μg/mL kanamycin for incubation overnight at 37° C. Plasmid was prepared from a single colony and the gene sequence confirmed by DNA sequencing.

TABLE 3
Primers used in this study.

| Gene name and | |
|---|---|
| organism | |
| (NCBI Accession#) | Forward (1^{st}) and reverse (2^{nd}) primer (5′→3′) |
| Glycerol kinase | 1. CATGGGCCGGCCACTTTCCCTCTCTCTTCCGACTTG |
| S. cerevisiae | (SEQ. ID 5) |
| (NP_011831) | 2. CATGCTCGAGTTATTGGAAGTTTTCTAGAACCTGTTCG |
| | (SEQ. ID 6) |
| Homoserine kinase | 1. CATGCATATGGTTCGTGCCTTCAAAATTAAAGTTC |
| S. cerevisiae | (SEQ. ID 7) |
| (NP_011890.1) | 2. CATGCTCGAGTTATCATTGCTGTTCGACGCTAG (SEQ. ID 8) |
| Phosphoribosyl- | 1. CATGCATATGCCTGATATGAAGCTTTTTGCTGG (SEQ. ID 9) |
| pyrophosphate | 2. CATGCTCGAGTTAGTGTTCGAACATGGCAGAGAT |
| synthase | (SEQ. ID 10) |
| E. coli | |
| (NP_415725.1) | |
| Homoserine kinase | 1. CATGCATATGGTTAAAGTTTATGCCCCGGCT (SEQ. ID 11) |
| E. coli | 2. CATGCTCGAGTTAGTTTTCCAGTACTCGTGCGC (SEQ. ID 12) |
| (ACX41206) | |
| Ethanolamine kinase | 1. CATGCATATGGCGCACGACGAACAATG (SEQ. ID 13) |
| E. coli | 2. CATGCTCGAGTTATCATTTTGCATATAGCCCCTCC (SEQ. ID |
| (CP001665.1) | 14) |
| Hydroxyethylthiazole | 1. CATGCATATGCAAGTCGACCTGCTGGGT (SEQ. ID 15) |
| kinase | 2. CATGCTCGAGTTAAGGAGGTGCAGGCATGA (SEQ. ID 16) |
| E. coli | |
| (ACT28613.1) | |
| Undecaprenol kinase | 1. CAGTAGATCTCAGCGATATGCACTCGCTG (SEQ. ID 17) |
| E. coli | 2. CAGTCTCGAGTTAAAAGAACACGACATACACCG (SEQ. ID |
| (ACA76318) | 18) |
| Diacylglycerol kinase | 1.CATGCATATGGCCAATAATACCACTGGATTCAC (SEQ. ID 19) |
| E. coli | 2. CATGCTCGAGTTATCCAAAATGCGACCATAAC (SEQ. ID 20) |
| (ACA79587.1) | |
| Glycerol kinase | 1.CATGCATATGACTGAAAAAAAATATATCGTTGCGC (SEQ. ID |
| E. coli | 21) |
| (ACT31081.1) | 2. CATGCTCGAGTTATTCGTCGTGTTCTTCCCAC (SEQ. ID 22) |
| 4-diphosphocytidyl- | 1. CCATGG CG ATGCGGACACAGTGGCCCTC (SEQ. ID 23) |
| 2-C-methyl-D- | 2. CTCGAGTTAAGCATGGCTCTGTGCAATGG (SEQ. ID 24) |
| erythritol kinase E. | |
| coli | |
| (AF179284) | |
| Farnesol kinase | 1. CGATGGATCCGATGGCAACTACTAGTACTACTACAAAGCTC |
| Arabidopsis thaliana | (SEQ. ID 25) |
| (NM_125242) | 2. CGATGCGGCCGCTTAGAAGAGTAAGAATCCGGCC (SEQ. ID |
| | 26) |
| Isopentenyl | 1. GTACAGATCTTATGCAAACGGAACACGTCAT (SEQ. ID 27) |
| diphosphate | 2. GTACCTCGAGTTAATTGTGCTGCGCGAAA (SEQ. ID 28) |
| isomerase idi from E. | |
| coli | |
| (cloned into | |
| pCDFDuet-GGPP) | |
| (BAE76954.1) | |
| IspA Y8OD from E. | 1. GTACCCATGGCGATGGACTTTCCGCAGCAAC (SEQ. ID 29) |
| coli | 2. GTACGCGGCCGCTTATTTATTACGCTGGATGATGTAGTCC |
| (cloned int | (SEQ. ID 30) |
| pCDFDuet-GGPP) | |
| (NP_414955) | |
| Phytoene desaturase | 1. GTACCCATGGGCATGAAACCAACTACGGTAATTGGTG (SEQ. |
| Crtl from Pantoea | ID 31) |
| ananatis | 2. GTACAAGCTTTTAAATCAGATCCTCCAGCATCAA (SEQ. ID |
| (cloned into | 32) |
| pACYCDuet-Lyc) | |
| (D90087) | |
| Phytoene synthase | 1. GTACAGATCTCATGGCAGTTGGCTCGAAAAG (SEQ. ID 33) |
| crtB from Pantoea | 2. GTACCTCGAGTTAGAGCGGGCGCTGC (SEQ. ID 34) |
| ananatis | |
| (cloned into | |
| pACYCDuet-Lyc) | |
| (D90087) | |
| Isopentenyl | 1.GTACCATATGATAGATCCGTTCACCATGATGATCC (SEQ. ID |
| phosphate kinase | 35) |
| (IPK) | 2. GTACCTCGAGTTAGCGAATCACGGTACCGAT (SEQ. ID 36) |
| Thermoplasma | |
| acidophilum | |
| (cloned in pETDuet) | |
| (CAC11251.1 | |
| Isopentenyl | 1. GTACCATATGGATCCGTTCACCATGATGATCC (SEQ. ID 37) |
| phosphate kinase | 2. GTACCTCGAGTTAGCGAATCACGGTACCGAT (SEQ. ID 38) |
| (IPK) | |
| Thermoplasma | |
| acidophilum | |
| (cloned in pET28a) | |
| (CAC11251.1) | |
| Non-specific acid | 1. CTAGGGATCCGATGAAACGTCAGCTGTTTACC (SEQ. ID 39) |
| phosphatase (PhoN), | 2. CTAGGCGGCCGCTTATTTTTTCTGATTATTGGCGAAT (SEQ. |
| Shigella flexneri | ID 40) |
| (BAA11655.1) | |
| Diacylglycerol kinase | 1. ATAGGATCCGATGCCGATGGATCTGC (SEQ. ID 41) |
| (DGK) | 2. CGATGCGGCCGCTTAATGAAACAGCAGAAACCAAATT |
| Streptococcus mutans | (SEQ. ID 42) |
| (AAN59259.1) | |
| Dimethylallyl | 1. ATACATATGAAAGCCGCAAACG (SEQ. ID 43) |
| tryptophan synthase | 2. TATAAGCTTATGCAGGCCGC (SEQ. ID 44) |
| (FgaPT2) | |
| Aspergillus fumigatus | |
| (cloned in pET28a) | |
| (AX08549.1) | |
| Dimethylallyl | 1. TATAGGATCCGATGAAAGCCGCAAACG (SEQ. ID 45) |
| tryptophan synthase | 2. ATATAAGCTTATGCAGGCCGC (SEQ. ID 46) |
| (FgaPT2) Aspergillus | |
| fumigatus (cloned in | |
| pCDFDuet) | |
| (AX08549.1) | |

Screening of Kinases for Phosphorylation of ISO and DMAA by LC-MS Analysis. Each plasmid containing a cloned kinase gene was transformed into chemically competent E. coli BL21(DE3) Tuner cells and plated on LB agar supplemented with 50 μg/mL kanamycin for incubation overnight at 37° C. Colonies were picked the following day and used to inoculate 3 mL LB supplemented with 50 g/mL kanamycin for incubation overnight at 37° C. A 1 mL portion of this culture was then used to inoculate 100 mL of LB supplemented with 50 μg/mL kanamycin and grown at 37° C. at 250 rpm until the culture reached OD₆₀₀ of ˜0.2 before the temperature was reduced to 18° C. and IPTG was added to a final concentration of 1 mM. The culture was incubated for 18 hours at 250 rpm. The culture was pelleted at 4,000 rpm for 10 min, the supernatant decanted, and the cell pellet resuspended in 5 mL of lysis buffer (100 mM Tris, 300 mM NaCl, 10% glycerol, pH 8.0) and lysed by sonication. The debris was then pelleted at 4,500 rpm for 20 min, decanted, and the soluble protein was spun down additionally at 15,000 rpm for 1 h. The resulting soluble fraction was then purified using loose Ni²⁺ resin from GE Healthcare. 200 μL of resin was added to the soluble fraction of protein and incubated on ice for 1 hour with intermittent agitation to suspend the resin. The resin was then spun down for 10 min at 4,500 rpm at 4° C. and the lysate was removed. The resin was then resuspended in 1 mL of wash buffer (50 mM Tris, 500 mM NaCl, 20 mM imidazole, pH 8.0) and transferred to a 1.5 mL tube. The mixture was allowed to incubate on ice for 10 min before the resin was spun down again as before. This washing procedure was repeated 4 more times before the protein was eluted with 200 μL of elution buffer (50 mM Tris, 500 mM NaCl, 200 mM imidazole, pH 8.0). The protein was then directly assayed and purity was verified by SDS-PAGE with comparison to the soluble fraction of E. coli BL21 (DE3) Tuner cells not harboring any plasmid. The assay consisted of 10 μL of purified protein in a total volume of 100 μL containing 5 mM ATP, 1 mM of ISO and DMAA (stock of 100 mM in DMSO), 50 mM Tris at pH 7.5, and 2.5 mM MgCl2. The reaction was incubated overnight at 37° C. before being quenched with an equal volume of methanol. The mixture was then analyzed by low-resolution LC-MS along with a synthetic standard of isopentenyl phosphate and dimethylallyl phosphate. LC-MS experiments were conducted using a Shimadzu LC-MS 2020 single quadrupole instrument with a Phenomenex Kinetex UPLC C18 column (2.1×50 mm, 2.6 μm particle, 100 Å pores) column. An aliquot (5 μL) was injected onto and separated using a series of linear gradients developed from 0.1% formic acid in H₂O (A) to 0.1% formic acid in acetonitrile (B) at 0.2 mL/min using the following protocol: 0-2.2 min, 95-1% A; 2.21-2.6 min, 1% A; 2.61-2.62 min, 1-95% A; 2.63-3.5 min, 95% A.

Synthesis of IPP and DMAPP. The synthesis of IPP and DMAPP followed a published procedure (Keller, R. K.; Thompson, R., Rapid synthesis of isoprenoid diphosphates and their isolation in one step using either thin layer or flash chromatography. Journal of chromatography 1993, 645 (1), 161-7). Briefly, to 4 mmol of the neat alcohol (IPO or DMAA) in a 50 mL polypropylene tube was added trichloroacetonitrile (10 mL) and the mixture was allowed to incubate at room temperature for 5 min. Bis-triethylammonium phosphate (TEAP) solution was prepared by slowly adding solution A (25 mL phosphoric acid, 94 mL acetonitrile) to solution B (110 mL triethylamine, 100 mL acetonitrile) to generate a solution that was 38% solution A and 62% solution B. To the mixture of alcohol and trichloroacetonitrile was added 10 mL of TEAP solution. The mixture was then incubated in a 37° C. water bath for 5 min before another addition of TEAP. A total of three additions of TEAP solution were added and incubated. The mixture was then separated by column chromatography using 6:2.5:0.5 iPrOH:conc. NH₄OH:H₂O with silica as the stationary phase. Prior to loading the column, the reaction mixture was diluted to 20% vol/vol with chromatography buffer and the resulting precipitate was pelleted by centrifugation prior to loading of the flash column. Fractions were analyzed by using a Shimadzu single quadrupole LCMS-2020 and those containing the diphosphorylated compound ([M−H]⁻), free of tri- or monophosphorylated were pooled. The pooled fractions were then concentrated in vacuo to remove isopropanol and acetonitrile. The concentrated mixture was then filtered using 0.2 m cellulose filter and frozen at −80° C. After being frozen overnight, the sample was lyophilized yielding a salt. The triammonium salt was then characterized and stored frozen in 250 μL aliquots at 25 mM each. Dimethylallyl diphosphate (DMAPP): ¹H NMR (400 MHz, D₂O) δ 5.43 (t, J=7.0 Hz, 1H), 4.43 (dd, J_{H,P}=7.0 Hz, J_{H,H}=7.0 Hz, 2H), 1.76 (s, 3H), 1.71 (s, 3H); ¹³C NMR (101 MHz, D₂O) δ 140.1, 119.7 (d, J_{C,P}=8.2 Hz), 62.7 (d, J_{C,P}=5.4 Hz), 25.0, 17.3; ³¹P NMR (162 MHz, D₂O) δ −6.04 (d, J=21.7 Hz), −9.38 (d, J=21.6 Hz); HRMS m/z calculated for C₅H₁₂O₇P₂ [M−H⁺]⁻ 244.9985, found: 244.9986. Isopentenyl diphosphate (IPP): ¹H NMR (400 MHz, D₂O) δ 4.02-3.92 (m, 2H), 2.30 (t, J=6.7 Hz, 2H), 1.68 (s, 3H); ¹³C NMR (101 MHz, D₂O) δ 143.8, 111.6, 64.1 (d, J_{C,P}=6.0 Hz), 37.9 (d, J_{C,P}=7.6 Hz), 21.7; ³¹P NMR (162 MHz, D₂O) δ −6.22 (d, J=21.6 Hz), −9.54 (d, J=21.5 Hz); HRMS m/z calculated for C₅H₁₂O₇P₂ [M−H⁺]⁻ 244.9985, found: 244.9985.

Preliminary Characterization of IPK via Lycopene Colorimetric Assay. Colonies of E. coli BL21Tuner(DE3) harboring pCDFDuet-GGPP (see below) and pACYCDuet-Lyc (see below) were used to inoculate separate wells of a deep-well plate containing 1 mL of LB media supplemented with ampicillin (150 μg/mL), chloramphenicol (35 μg/mL), and streptomycin (200 μg/mL). The deep-well plate was incubated overnight in a rotary shaker at 37° C. with orbital shaking at 350 rpm. Then, 50 μL of the culture was used to inoculate 400 μL of LB media supplemented with ampicillin (120 μg/mL), chloramphenicol (28 μg/mL), and streptomycin (160 μg/mL). After 3 h of incubation at 37° C. at with shaking at 350 rpm, the OD₆₀₀ of the culture was approximately 0.1, and IPTG, DMAA/ISO, and Fs were each added to give final concentrations of 1 mM, 5 mM, and 0.5 μM, respectively, to bring the cultures to a final volume of 500 μL. For controls that lacked one or more of these components, LB media was added instead. Plates were then incubated in the dark in an incubator/shaker at 30° C. with shaking at 350 rpm for 48 h. Then, the deep-well plate was centrifuged at 3,000 rpm for 7 min to pellet the cells. After removal of the growth media from each well, the pellets were resuspended in 1 mL of phosphate buffer saline buffer with vigorous vortexing and the resuspended pellets were visualized and photographed.

Construction of pCDFDuet-GGPP and pACYCDuet-Lyc. pCDFDuet-GGPP contained a mutated version of the E. coli IspA gene (Y80D) and idi from E. coli that were subcloned sequentially into pCDFDuet. Briefly, ispA and idi were each PCR amplified from E. coli DH5α using the primers listed in Table 3. Each PCR reaction mixture contained 5× Phire II buffer, 0.2 mM dNTPs, 0.5 μM each primer, 1 μL Phire II DNA polymerase, and 1 μL of template DNA in a total volume of 50 μL. The cycling parameters used were as follows: 1) 98° C., 30 s; 2) 98° C., 5 s; 3) 63° C., 15 s; 4) 72° C., 20 s; 5) repeat steps 2-4 34 times; 6) 72° C., 1 min; 7) 4° C., hold. Following amplification, the products were purified by gel electrophoresis and digested with NcoI and Nod for ispA and BgIII for idi and ligated into the appropriately treated pCDFDuet vector. Each ligation mixture was transformed into chemically competent E. coli DH5α and plated on an LB agar plate containing 200 μg/mL streptomycin. Plasmid was prepared from a single colony and the gene sequence confirmed by DNA sequencing. The mutation Y80D was introduced by site-directed mutagenesis.

pACYCDuet-Lyc contains the CrtEBI operon genes from Pantoea ananatis that were sub-cloned sequentially using pACmod-crtE-crtB-crtl as a template. Briefly, crtB and crtI were each PCR amplified from the template pACmod-crtE-crtB-crtI using the primers listed in Table 3. Each PCR reaction mixture contained 5× Phire II buffer, 0.2 mM dNTPs, 0.5 μM each primer, 1 μL Phire II DNA polymerase, and 1 μL of template DNA, in a total volume of 50 μL. The cycling parameters used were as follows: 1) 98° C., 30 s; 2) 98° C., 5 s; 3) 63° C., 15 s; 4) 72° C., 20 s; 5) repeat steps 2-4 34 times; 6) 72° C., 1 min; 7) 4° C., hold. Following amplification, the products were purified by gel electrophoresis and digested with BgIII and XhoI for crtB and NcoI and HindIII for crtI and ligated into the appropriately treated pCDFDuet vector. Each ligation mixture was transformed into chemically competent E. coli DH5α and plated on an LB agar plate containing 35 μg/mL chloramphenicol. Plasmid was prepared from a single colony and the gene sequence confirmed by DNA sequencing.

Cloning of IPK, PhoN, and DGK. The sequence of ipk (Table 2) codon-optimized for heterologous expression in E. coli was synthesized by IDT and PCR amplified using the primers listed in Table 3. The PCR reaction mixture contained 5× Phire II buffer, 0.2 mM dNTPs, 0.5 μM each primer, 1 μL Phire II DNA polymerase, and 1 μL of template DNA, in a total volume of 50 μL. The cycling parameters used were as follows: 1) 98° C., 30 s; 2) 98° C., 5 s; 3) 63° C., 15 s; 4) 72° C., 20 s; 5) repeat steps 2-4 34 times; 6) 72° C., 1 min; 7) 4° C., hold. Following amplification, the product was purified by gel electrophoresis, digested with NdeI and XhoI and ligated into similarly treated pETDuet-1 (into multi-cloning site two). The ligation mixture was transformed into chemically competent E. coli DH5α and plated on LB agar containing 100 μg/mL of ampicillin. Plasmid was prepared from a single colony and the gene sequence confirmed by DNA sequencing using the primer ‘DuetUP2’ (5′-TTGTACACGGCCGCATAATC-3′; SEQ. ID 47). For cloning into pET28a, the ipk gene was PCR amplified using the primers listed in Table 3 and the same PCR conditions as described above. Following amplification, the product was purified by gel electrophoresis and was digested with NdeI and XhoI and ligated into similarly treated pET28a. The ligation mixture was then transformed into chemically competent E. coli DH5α and plated on LB agar containing 50 μg/mL kanamycin. Plasmid was prepared from a single colony and the gene sequence confirmed by DNA sequencing.

The sequence of phoN (Table 2) codon-optimized for expression in E. coli was synthesized by IDT and PCR amplified using the primers listed in Table 2. The PCR reaction contained 5× Phire II buffer, 0.2 mM dNTPs, 0.5 μM each primer, 1 μL Phire II DNA polymerase, and 20 ng of template DNA, in a total volume of 50 μL. The cycling parameters used were as follows: 1) 98° C., 30 s; 2) 98° C., 5 s; 3) 66° C., 15 s; 4) 72° C., 20 s; 5) repeat steps 2-4 34 times; 6) 72° C., 1 min; 7) 4° C., hold. Following amplification, the amplified product was gel purified, digested with BamHI and NotI, and ligated into similarly treated ‘empty’ pETDuet and pETDuet-IPK (into MCS1). The ligation mixture was transformed into chemically competent E. coli DH5α and plated on LB agar containing 100 μg/mL of ampicillin. Plasmid was prepared from a single colony and the gene sequence confirmed by DNA sequencing using the primer ‘pET Upstream’ (ATGCGTCCGGCGTAGA; SEQ. ID 48).

The sequence of DGK from Streptococcus mutans codon optimized for expression in E. coli (Table 2) was synthesized and subcloned into pET28a according to the same protocol for cloning candidate kinase genes above, using the primers listed in Table 3.

Expression and Protein Purification of PhoN. pETDuet-PhoN was transformed into chemically competent E. coli BL21(DE3) for protein expression. An overnight 3 mL culture in LB broth of PhoN-pETDuet containing 100 μg/mL of ampicillin was grown at 37° C. and 270 rpm. A 1 L culture in LB broth was inoculated with 1 mL of the overnight culture and grown at 37° C. and 250 rpm to an OD₆₀₀ of 0.6. The culture was cooled to 18° C., and protein expression was induced by the addition of IPTG to a final concentration of 1 mM. The culture was incubated at 18° C. and 200 rpm for an additional 20 h. The culture was spun down into a pellet which was stored at −20° C. until purification. The cell pellets were thawed and resuspended in 20 mL of lysis buffer (250 mM sodium chloride, 50 mM sodium phosphate, 10 mM imidazole, pH 7.9). The cells were lysed by sonication and spun down. The cell lysate was then separated from the insoluble cell debris, and the His₆-tagged proteins were purified from the lysate using Ni²⁺ beads on agarose using a low-imidazole buffer (50 mM Tris, 300 mM NaCl, 20 mM imidazole, pH 8.0) as the wash buffer and a high-imidazole buffer (50 mM Tris, 300 mM NaCl, 200 mM imidazole, pH 8.0) to isolate the protein. Then, a 10 kDa spin filter was used to concentrate and buffer exchange the protein into storage buffer (50 mM Tris-HCl, 500 mM NaCl, 20% glycerol, pH 7.4). The concentration of protein was determined using a Bradford assay, and small aliquots of protein were stored at −80° C. until needed.

Expression and Protein Purification of IPK. pET28a-IPK was transformed into chemically competent E. coli BL21(DE3) for protein expression. An overnight 3 mL culture in LB broth supplemented with kanamycin (50 μg/mL) was grown at 37° C. with shaking at 270 rpm. A 1 L culture in LB broth was inoculated with 1 mL of the overnight culture and grown at 37° C. with shaking at 250 rpm to an OD₆₀₀ of 0.6. The culture was cooled to 30° C. and protein expression was induced by the addition of IPTG to a final concentration of 1 mM. The culture was incubated at 30° C. and 200 rpm for an additional 20 h. The culture was spun down into a pellet which was stored at −20° C. until purification. Cell pellets were thawed and resuspended in 25 mL lysis buffer (250 mM sodium chloride, 50 mM sodium phosphate, 10 mM imidazole, pH 7.9). The cells were lysed by sonication and centrifuged. The cell lysate was then separated from the insoluble cell debris, and the His₆-tagged proteins were purified from the lysate using the Bio-Rad Profinia system and Bio-Scale Mini Nuvia IMAC Ni-Charged 5-mL columns. Following loading of the sample onto the column, the system washed first with 6 column volumes of 2× Native IMAC Wash 1 solution (1 M NaCl, 100 mM Tris, 10 mM imidazole, pH 8.0) and then with 6 column volumes of 2× Native IMAC Wash 2 solution (1 M NaCl, 100 mM Tris, 40 mM imidazole, pH 8.0). The sample was eluted in 3 column volumes of 2× Native IMAC Elution buffer (1 M NaCl, 100 mM Tris, 500 mM imidazole, pH 8.0). Then the eluent was concentrated and buffer exchanged into protein storage buffer (50 mM Tris-HCl, 500 mM NaCl, 20% glycerol, pH 7.4) using 10 kDa molecular-weight cutoff filters. The concentration of protein was determined using a Bradford assay and small aliquots of protein were stored at −80° C. until needed.

Lycopene quantification. E. coli NovaBlue (DE3) containing pAC-LYCipi and various pETDuet constructs were grown in 250 mL LB supplemented with ampicillin (100 μg/mL) and chloramphenicol (35 μg/mL) at 37° C. overnight with shaking at 250 rpm after inoculation with 0.25 mL of the starter culture. After 5 h the OD₆₀₀ of the culture was ˜0.2 at which point combinations of DMAA/ISO (in DMSO), IPTG, and Fs were added to give final concentrations of 5 mM, 1 mM, and 0.5 μM, respectively. In controls that lacked DMAA/ISO, DMSO was added to give the equivalent volume. At various time points, 600 μL of culture was then removed and the lycopene was extracted and quantified.

Extraction and Quantification of Lycopene. Each aliquot (600 μL) of culture was subjected to acetone extraction and quantification of lycopene by HPLC. The remaining 500 μL was centrifuged at 10,000 rpm, and the supernatant removed. The cell pellets were then dried using a speed vacuum without heat until the pellets were dry. Then, 200 μL of acetone was added to the pellets, and the tubes were sonicated at 37° C. for 20 min before incubation at 55° C. for 30 min. The tubes were then sonicated again as before. The pellets were spun down and 100 μL was removed for HPLC analysis. HPLC was performed by injecting 10 μL of the clarified extract onto a Phenomenex Kinetex EVO C18 column (250×4.6 mm, 5 μm, 100 Å pores) with an isocratic elution buffer consisting of 8:1.5:0.5 isopropanol:acetonitrile:methanol over 20 min. Lycopene was assayed at 470 nm. Areas were extracted and compared to the standard curve for quantification.

Lycopene Standard Curve. A standard curve for lycopene was derived by adding various known amounts of commercial lycopene standard to E. coli Novablue(DE3) cell pellets, extracting, and quantified as outlined above.

Cloning of FgaPT2. The sequence of fgaPT2 codon-optimized for expression in E. coli (Table 2) was synthesized by IDT and PCR amplified using the primers listed in Table 3. The 50 μL reaction for amplification of fgaPT2 contained 5× Phire buffer, 0.2 mM dNTPs, 0.25 μM each primer, 1 μL Phire II DNA polymerase, and 1 μL template DNA. The cycling parameters used for each were as follows: 1) 98° C., 30 s; 2) 98° C., 5 s; 3) 64° C., 15 s; 4) 72° C., 20 s; 5) repeat steps 2-4 34 times; 6) 72° C., 1 min; 7) 4° C., hold. Following PCR amplification, the amplified product was purified by gel electrophoresis, digested with HindIII and NdeI and ligated into similarly treated pET28a to generate pET28a-FgaPT2. The ligation mixture was then transformed into chemically competent E. coli DH5α and plated on LB agar containing 50 μg/mL of kanamycin. Plasmid was prepared from a single colony and the gene sequence confirmed by DNA sequencing using the T7 promoter primer. For cloning into pCDFDuet (used in conjunction with the ADH module in pETDuet-PhoN-IPK), fgaPT2 was PCR amplified using the same conditions as above but with different primers also listed in Table 3. Following amplication, the product was digested with BamHI and HindIII and ligated into similarly treated pCDFDuet. The ligation mixture was then transformed into chemically competent E. coli DH5α and plated on LB agar plates containing 100 μg/mL of streptomycin. Plasmid was prepared from a single colony and the gene sequence confirmed by DNA sequencing using the DuetUP2 and T7 terminator primers.

Expression and Protein Purification of FgaPT2. pET28a-FgaPT2 was transformed into chemically competent Rossetta PLysS cells for expression. An overnight 3 mL culture of these cells containing 50 μg/mL of kanamycin and 25 μg/mL of chloramphenicol in LB media was grown at 37° C. and 270 rpm. A 1 L culture in terrific broth containing 30 μg/mL of kanamycin and 35 μg/mL of chloramphenicol was inoculated with 2 mL of overnight culture and grown at 37° C. and 250 rpm to an OD₆₀₀ of 0.6. Once OD was reached, the culture was cooled to 24° C. and induced by the addition of IPTG to 0.5 mM final concentration. The culture was incubated for 24 h at 24° C. The culture was pelleted and stored in two aliquots at −20° C. until purification. Cell pellets were thawed and resuspended in 25 mL lysis buffer (250 mM sodium chloride, 50 mM sodium phosphate, 10 mM imidazole, pH 7.9). The cells were lysed by sonication and spun down. The cell lysate was then separated from the insoluble cell debris, and the His₆-tagged proteins were purified from the lysate using the Bio-Rad Profinia system and Bio-Scale Mini Nuvia IMAC Ni-Charged 5-mL columns. Following loading of the sample onto the column, the system washed first with 6 column volumes of 2× Native IMAC Wash 1 solution (1 M NaCl, 100 mM Tris, 10 mM imidazole, pH 8.0) and then with 6 column volumes of 2× Native IMAC Wash 2 solution (1 M NaCl, 100 mM Tris, 40 mM imidazole, pH 8.0). The sample was eluted in 3 column volumes of 2× Native IMAC Elution buffer (1 M NaCl, 100 mM Tris, 500 mM imidazole, pH 8.0). Then the eluent was concentrated and buffer exchanged into protein storage buffer (50 mM Tris-HCl, 500 mM NaCl, 20% glycerol, pH 7.4) using 50 kDa molecular-weight cutoff filters. The concentration of protein was determined using a Bradford assay, and small aliquots of protein were stored at −80° C. until needed.

In vitro FgaPT2 assay. FgaPT2 reactions were run at pH 7.5 in 200 μL containing 50 mM Tris-HCl, 5 mM CaCl₂), 1 mM L-tryptophan, 2 mM DMAPP, and 40 g of FgaPT2. The reactions were incubated at 37° C. for 1 h and then quenched by the addition of an equal volume of methanol. Reactions with PhoN-IPK generated DMAPP contained 25 mM Tris-HCl, 5 mM magnesium chloride, 1 mM L-tryptophan, 1.8 mM ATP, 30 mM DMAA, 270 ng/μL FgaPT2, 20 ng/μL IPK, and 87 ng/μL PhoN at pH 8.0 in a total volume of 50 μL. The reactions were incubated at 37° C. overnight and then quenched by the addition of an equal volume of methanol. For analytical-scale HPLC analysis, FgaPT2 reactions were followed at 269 nm using a Phenomenex Kinetex 5u EVO C18 column (250×4.6 mm; 100 Å) at a flow rate of 1 mL/min. A linear gradient of 20-70% acetonitrile in 0.1% aqueous trifluoroacetic acid over 20 min was used.

In vivo FgaPT2 assay. A 3 mL culture of E. coli Rosetta(DE3) pLysS pETDuet-PhoN-IPK+pCDFDuetFgaPT2 was grown overnight at 37° C. and 250 rpm in LB media containing ampicillin (100 μg/mL), chloramphenicol (35 μg/mL), and streptomycin (100 μg/mL). An aliquot (100 μL) of overnight culture were used to inoculate 10 mL cultures in TB media containing the same antibiotics as before, and those cultures were grown at 30° C. for 6 h at 250 rpm until induction with 0.5 mM IPTG (final concentration) and addition to a final concentration of 5 mM DMAA, 5 mM ISO, and 10 mM Trp. The cultures were grown for 48 h after induction. The culture supernatant was diluted 1:1 in methanol before analysis by HPLC and LC-MS.

### Example 3. Probing the Substrate Promiscuity of Isopentenyl Phosphate Kinase as a Platform for Hemiterpene Analogue Production

Unnatural linear terpene precursors with enhanced chemical diversity beyond a carbon-hydrogen scaffold would expand the scope of available chemo-, regio-, and stereo-specific organic transformations that could be used to diversify terpene scaffolds generated by cyclases. In addition, such unnatural analogues may provide uncharacterized modes of reactivity for terpene cyclases. Importantly, such unnatural linear terpene precursors could also be appended to other natural products such as meroterpenoids and ergot alkaloids.

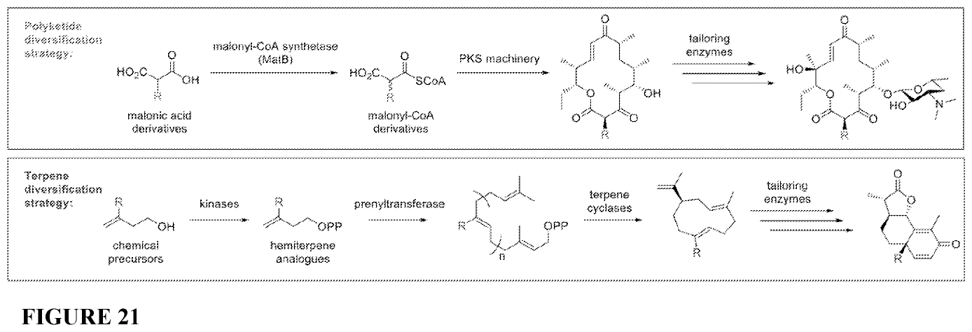

The generation of unnatural terpenes through, for example, a mixed synthetic biology and precursor directed diversification strategy, would provide chemists previously unavailable chemical handles needed for synthetic derivatization for use in structure-activity relationships, pharmaceutical production, and biochemical studies. A similar strategy can also explored for the production of unnatural biosynthetic polyketides (FIG. 21)

Both terpene synthases and terpene cyclases have already been shown to be at least partially promiscuous towards analogues of their natural substrates. A platform enabling the production of hemiterpene analogues would allow for generation of diversified building blocks for which prenyltransferases and terpene cyclases could be engineered to accept.

Terpene precursors are naturally synthesized by the DXP and mevalonate pathways. Terpenes, produced in E. coli by the DXP pathway, are essential for the construction of lipid carriers used in the transportation of glycan components for the maintenance of the cell envelope. Because terpenes are essential for cell survival, modification of the native anabolic pathway may be lethal and in addition, would require the engineering of up to 7 enzymes and methodical planning of how to generate their corresponding substrate analogues from primary metabolites.

In an attempt to generate unnatural hemiterpenes, the lower part of the mevalonate pathway was investigated for plasticity by providing chemically synthesized analogues for sequential biocatalytic conversions. However, these substrates aren't commercially available and require extensive synthesis (9 steps minimum, 4 steps after divergence, 8 chromatographic purifications, 47% overall yield maximally achieved, and racemic). Analogues produced from such a route can only contain the homoallylic diphosphate core of IPP and would result in minimal and restricted modifications to IPP (Schemes 1 and 2). This limited flexibility for analogue generation is a consequence of using biocatalysts that have stereochemical preferences in addition to the requirement of structural motifs essential for full substrate maturation.

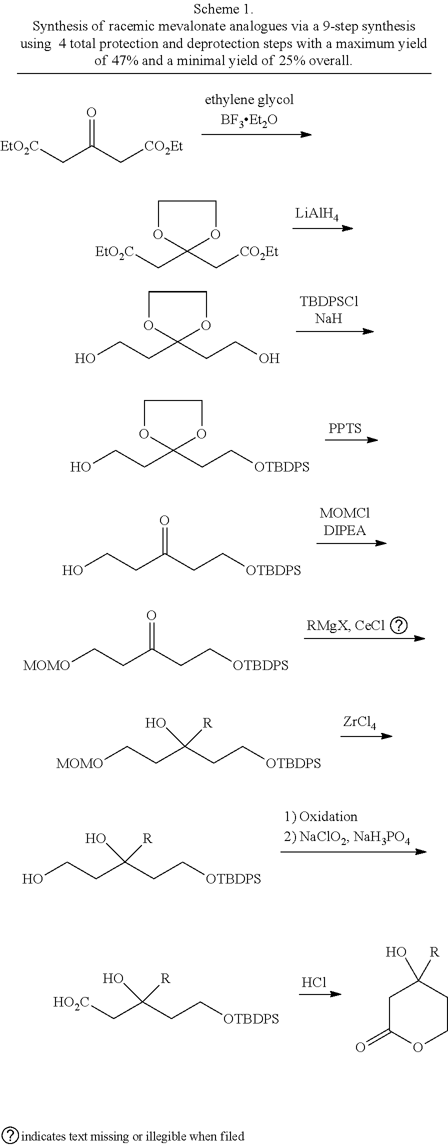

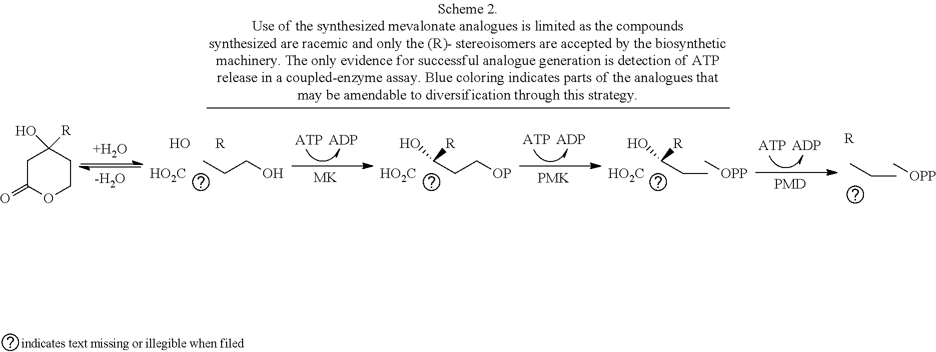

As an alternative to engineering endogenous metabolism to accept structural analogues of their native substrates, unnatural hemiterpenes can be produced by consecutive enzymatic phosphorylation of alcohols, as is accomplished synthetically. Use of an artificial pathway for the generation of natural hemiterpenes from the corresponding alcohols of IPP and DMAPP can be accomplished by the use of PhoN and IPK (Example 2). Instead of supplementing this novel pathway with a carbon precursor that would be converted to natural terpenes, it was envisioned that this pathway could potentially be used to generate hemiterpene analogues by providing the pathway with an alternative substrate.

Conversion of the alcohols to the corresponding hemiterpene analogues would require just two equivalents of ATP, a small energy cost compared to that of the mevalonate pathway (3×ATP and 2×NADH) and the DXP pathway (1×ATP, 1×CTP, and 3×NADH). As hemiterpene production via the mevalonate and DXP pathways often limits downstream production of terpenes, the energy benefit alone justifies inquiry of a novel biosynthesis platform.

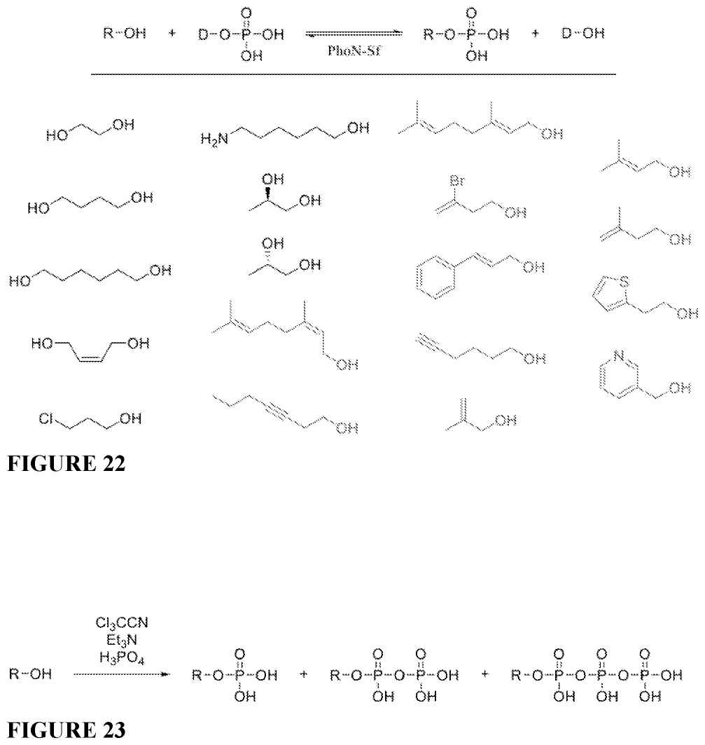

A two-step biosynthetic platform using a non-specific acid phosphatase (PhoN) from Shigella flexneri and IPK from Thermoplasmsa acidophilum was validated in Example 2 by demonstrating that the production of carotenoids in an engineered E. coli reporter strain was dependent on feeding isopentenol (ISO) and dimethylallyl alcohol (DMAA). Importantly, both component enzymes exhibit significant substrate promiscuity. PhoN can phosphorylate a wide variety of alcohols with various phosphate donors (FIG. 22). Minimal studies have been conducted to describe promiscuity of IPK towards various alkyl phosphates that may be relevant to terpene diversification. The objective of this example is to fully explore the substrate promiscuity of IPK by defining its scope and utility as a tool to generate non-natural terpene precursors. It is hypothesized that IPK will display broad specificity towards a wide range of alcohol monophosphates. If so, this can be effectively coupled with an upstream candidate isopentenol kinase (e.g., PhoN) to produce non-natural hemiterpenes.

Results and Discussion

Design and synthesis of a hemiterpene monophosphate analogue library. In order to describe the substrate promiscuity of IPK, a panel of isopentenyl monophosphate analogues was designed and synthesized (FIG. 23). The panel of analogues was designed to have various alkyl chain lengths, branching, substituents, and hybridization in order to characterize the catalytic flexibility of IPK.

The most straightforward and robust method was initially developed by Cramer before being modified by Keller and Thompson. This modified synthesis did not need to be carried out anhydrously, and a single purification step could be used to isolate the desired compounds. While this reaction was simpler to carry out for the production of many analogues, the reaction produces a mixture of the corresponding monophosphate, diphosphate, and triphosphate. This chemistry was carried out with starting materials that had a single alcohol in order to eliminate the isolation and purification of multiple regio-isomers. Building blocks containing other nucleophiles besides a single alcohol were omitted as the phosphorylation reaction would have produced mixed phosphorylation patterns

Alcohols were mixed with trichloroacetonitrile before addition the addition of triethylammonium phosphate solution (TEAP). After TEAP was added, the mixture was incubated at 37° C. for a few minutes before another addition of TEAP was added and the process repeated for a total of three additions. Next, the mixture was diluted by adding 20% v/v modified chromatography buffer to precipitate insoluble contaminants. The mixture was then centrifuged to pellet the contaminants before the mixture was loaded onto a silica column for separation. Initial iterations of the column chromatography were conducted in 60 mL syringes before being transferred to a flash column for larger scale syntheses. Syntheses were optimized for isolation of the diphosphates, however the monophosphate containing fractions could be collected. Initially, monophosphate fractions were identified by MS and concentrated in vacuo. While the resulting residue was not pure, the residues were diluted to 5 mg/mL and IPK was tested with and without ATP to observe whether the compounds were indeed substrates. If sufficient activity was observed, the syntheses were scaled for isolation of the monophosphates.

Because the chromatography in the syringes has a limited flowrate due to elution being dictated by gravity, this chromatographic separation takes more than eight hours and typically yields ˜15 mg of product. After scaling the reaction 20-fold, removing precipitate to increase flow rates, and using flash columns to hold larger volumes of reaction mixtures as well as pressure to increase flow rates, upwards of 150 mg of monophosphate could be isolated with the synthesis and chromatography taking a total of 2.5 hours.

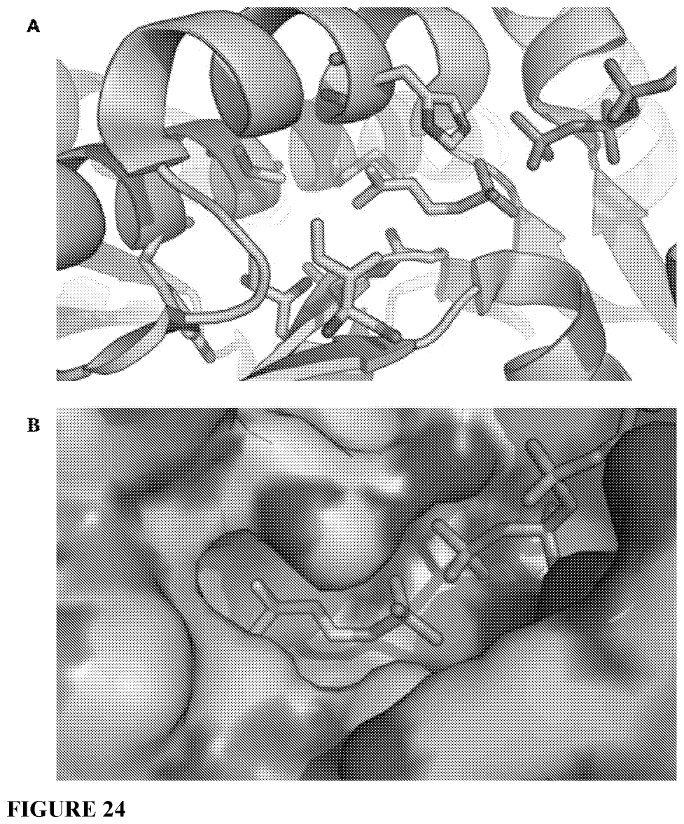

Characterization of the substrate promiscuity of isopentenyl phosphate kinase from Thermoplasma acidophilum. Recently, several isopentenol phosphate kinases (IPK) from archaea have been characterized. In addition to a crystal structure being available, engineering a wider substrate tolerance of IPK from Thermoplasma acidophilum had been successful for the phosphorylation of geranyl monophosphate and farnesyl monophosphate. It was envisioned that this successful rational approach could be used to broaden substrate specificity if the catalytic use of IPK was found to be limiting. Several pieces of evidence suggest that IPK could be successfully engineered to broaden its substrate specificity. For example, the kinetic parameters and optimal conditions for IPK from T. acidophilum have already been determined by others, and the enzyme has been found to have the highest k_{cat}/Kₘ at pH 7.5 and was stable up to 70° C. The thermostability suggests a rigid structure that are often amenable to engineering. Further, IPK was also found to be active towards a variety of C4 and C5 monophosphorylated substrates. After finding some basal level of geranyl monophosphate (GP) phosphorylation, IPK has also been engineered to use GP resulting in 130-fold increase in k_{cat}/Kₘ at the cost of specificity with IP. As the authors simply used structure-guided alanine scanning mutagenesis to identify this mutant, further engineering could result in an increase of k_{cat}/Kₘ toward longer length terpene precursors (FIG. 24).

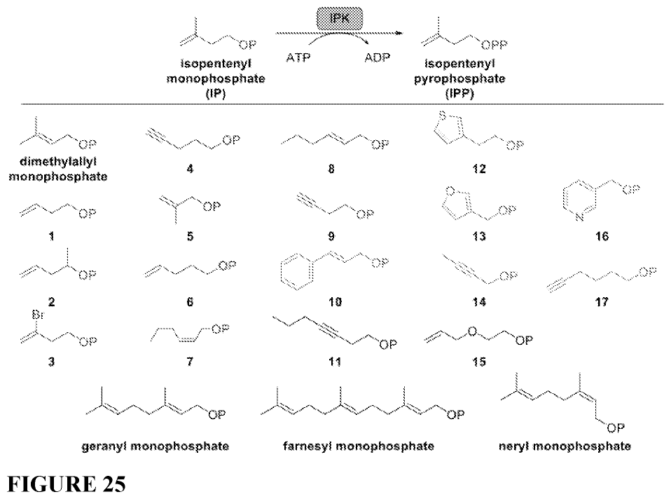

Since the substrate scope of IPK has yet to be fully described, here IPK was tested against a wider panel of substrates. The IPK gene from T. acidophilum was codon optimized and subcloned into pET28a. Following expression in E. coli BL21 DE3, the enzyme was purified via metal-chelation affinity chromatography. Next, the substrate specificity of the enzyme was determined in vitro using a panel of synthesized alcohol monophosphates using low resolution mass spectrometry (FIG. 25). The availability of alcohol diphosphate byproducts (FIG. 23) conveniently served as product standards.

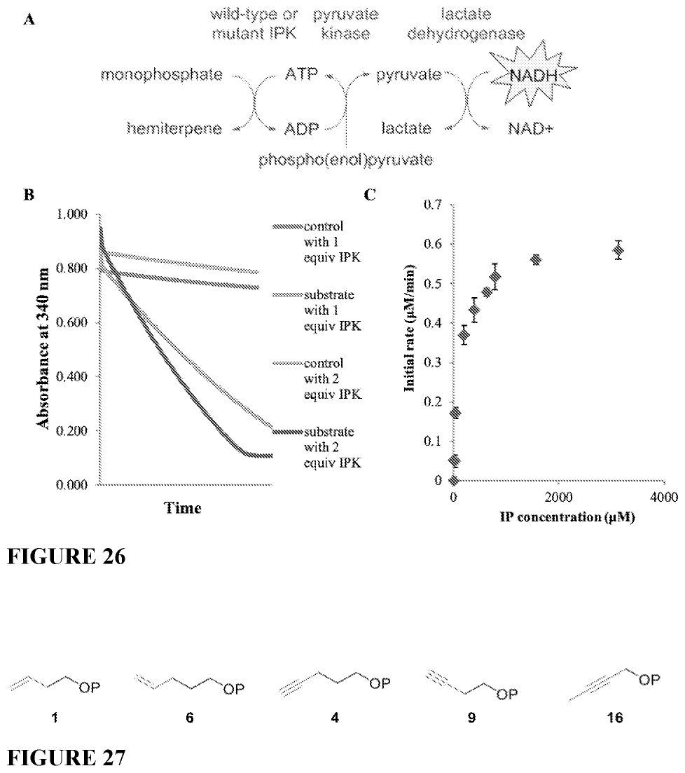

This initial study revealed broad promiscuity of IPK towards a wide variety of substrates. IPK showed some activity with nearly every substrate tested, while 11, 15, geranyl monophosphate (GP), FP, and neryl monophosphate (NP) did not result in detection of the corresponding pyrophosphate, as judged by MS analysis. Substrates 10, 12, 13, 14, 16, and 17 supported detectable levels of phosphorylations as found by MS, but as the conversions were very low after overnight reactions, they were deemed too poor for subsequent kinetic analysis. These results suggest that substrate promiscuity is limited by simple sterics, whereby substrates longer than 17 were not accepted by the enzyme. With the exception of substrate 14, all of the smaller compounds were substrates for IPK. To better understand the limits and utility of IPK for these phosphorylations, kinetics parameters required determination with a wide variety of representative substrates. A simple moderate throughput microplate assay was employed for this purpose. To examine the kinetic parameters of IPK towards novel substrates, a commonly used NADH-coupled assay was employed (FIG. 26).

Steady stake kinetic parameters of the IPK-catalyzed turnover of successful substrates were determined by measuring initial rates using a fixed concentration of phosphate donor and variable concentration of alcohol monophosphate (Table 4, see methods for details). The data was fitted to the Michaelis-Menten equation using SigmaPlot, and the kinetic parameters (k_{cat}, Kₘ, k_{cat}/Kₘ) extracted.

TABLE 4
Kinetic parameters of IPK with monophosphorylated substrates. Values are ± SD of the mean.

| | | | | k_{cat}/Kₘ |
|---|---|---|---|---|
| Structure | Substrate | Kₘ (μM) | k_{cat} (s⁻¹) | (M⁻¹ s⁻¹) |
| 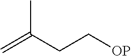 | IP | 27.9 ± 4.37 | 43.9 ± 1.10 | 1.5 × 10⁶ |
| 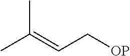 | DMAP | 134 ± 38.2 | 32.7 ± 2.05 | 2.3 × 10⁵ |
| 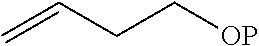 | 23 | 578 ± 18.0 | 187 ± 2.16 | 3.2 × 10⁵ |
| 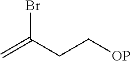 | 24 | 282 ± 64.1 | 225 ± 14.9 | 8.0 × 10⁵ |
| 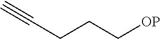 | 25 | 1400 ± 232 | 49.2 ± 3.99 | 3.5 × 10⁴ |
| 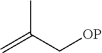 | 26 | 2030 ± 2.24 | 14.6 ± 3.56 | 7.2 × 10³ |
| 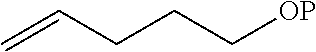 | 27 | 848 ± 57.9 | 93.1 ± 2.67 | 1.1 × 10⁵ |
| 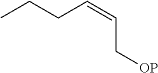 | 28 | 1090 ± 68.8 | 116 ± 3.31 | 1.1 × 10⁵ |
| 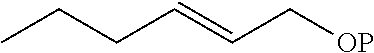 | 29 | 6830 ± 3770 | 43.0 ± 17.8 | 6.3 × 10³ |
| 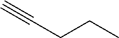 | 30 | 3020 ± 667 | 47.9 ± 6.47 | 1.6 × 10⁴ |

Gratifyingly, the Kₘ and k_{cat} values were within 10% of previously reported kinetic constants for T. acidophilum IPK with IP, DMAP, and 1. Due to limits in solubility for many of the substrates, kinetic values weren't obtained for a large portion of the substrates initially found using LC-MS (10, 12, 13, 14, 16, and 17). Notably, IPK displayed a higher k_{cat} with many of the substrates compared to the natural substrate, IP. For example, the k_{cat} with 1 and 3 was 4.25- and 5.1-fold higher than that with IP. However, this increase in k_{cat} was offset by the large increase in Kₘ which resulted in lower catalytic efficiencies (k_{cat}/Kₘ) for all non-natural substrates tested when compared to the natural substrate IP. While Kₘ isn't a perfect descriptor for affinity, the analogues tested all had significantly higher Kₘ's presumably due to IPK having a high specificity for IP at low concentrations. This could be a mechanism by which IPK only phosphorylates IP instead of any monophosphorylated metabolite in the cellular context. Interestingly, and consistent with previous data, some correlation is observed between overall length of the substrate and catalytic efficiency. For instance, there was a 3-fold drop in k_{cat}/Kₘ between 1 and 6. Branching at C2 also seems to dramatically lower k_{cat}/Kₘ as observed with 5 and the detectable but kinetically irrelevant use of 2. Interestingly, a 17-fold decrease in k_{cat}/Kₘ is observed when the π-bond geometry of 7 is switched from Z to E in substrate 8. Overall, longer substrates and especially those with structural rigidity (high proportions of sp² and sp) are poorer substrates for phosphorylation by IPK (FIG. 27)

As diphosphate moieties are presumably the primary component contributing to the binding energy between prenyltransferases and these short alkyl monophosphates, it makes sense that IPK has a wide substrate tolerance. Prediction of c Log P values of the substrates to measure greasiness and plotting these against measured Kₘ's and k_{cat}'s provided no correlation. No correlation between Kₘ's and k_{cat}'s were observed when plotting against molecular volumes. While IPK exhibits a wide substrate tolerance, Kₘ values very greatly. This indicates that while it was hypothesized that the phosphate was the primary governing force in substrate binding, the remaining alkyl portions of the monophosphates have a large impact on Kₘ. Residues found to allow for the turnover of larger substrates are not involved in other aspects of catalysis and seem to sterically accommodate larger alkyl chains (FIG. 24). The active site may be occupied by water and substrate binding might displace water for favored lipophilic interactions. Besides the phosphate moiety on these analogues, the enzyme simply has to allow for a larger, greasier substrate. Consistent with reported findings, the lower catalytic efficiency of IPK with small monophosphorylated substrate analogues isn't due to k_{cat}, but is attributable to higher Kₘ values. Several examples of expanding the substrate tolerance of IPK towards longer natural prenyl monophosphates has been achieved. In these successes, the authors note that while expansion of the substrate binding pocket increased k_{cat} but did not substantially alter Kₘ for the longer chain phosphates, a larger Kₘ was observed for these mutants with IP. The authors hypothesize that this expansion of the active site allows for IP to bind in more unproductive conformations either due to a looser binding pocket or due to the presence of additional water molecules.

Conclusions and Future Work

The data in this example points towards the plausibility of using IPK to generate small monophosphorylated substrates (from four up to eight carbons) into their corresponding pyrophosphates. Congruent with the promiscuity of PhoN, such a system can be coupled together to convert a broad variety of alcohols into diphosphorylated compounds via the consumption of just two phosphate donors. Notably, Nature has not been afforded the opportunity to select against the use of 1-15 as substrates, and this is effectively leveraged by PhoN-IPK as a platform the hemiterpene production.

In order to generate hemiterpene analogues for non-naturally prenylated compounds and terpene natural product derivatives, the next step is to couple these enzymes in vivo.

Methods

General. All plasmids were verified by DNA sequencing. Purifications of all DNA were performed with kits from BioBasic. Synthetic oligonucleotides were purchased from IDT (Coralville, Iowa, USA). All plate reader assays were performed using a BioTek Hybrid Synergy 4 plate reader (Winooski, Vt., USA). Restriction enzymes were purchased from New England Biolabs (Ipswich, Mass., USA). Polymerase chain reactions were conducted using Phire Hot Start II DNA Polymerase from ThermoFisher Scientific (Waltham, Mass., USA). Chemicals were purchased from Sigma Aldrich (St. Louis, Mo., USA) and Alfa Aesar (Haverhill, Mass., USA).

Gene Cloning. Isopentenyl monophosphate kinase (IPK) from Thermoplasma acidophilum was codon-optimized and synthesized by Genewiz, Inc. The ipk gene was PCR amplified from the provided template using then cloned into pET28a using NdeI and XhoI restriction sites. PCR was performed using Phire Hot Start II polymerase (ThermoFisher) according to supplier's protocol. PCR product was purified prior to and after digestion by agarose gel electrophoresis. Digested PCR product and similarly treated pET28a were ligated at room temperature with T4 ligase (New England BioLabs) according to supplier's protocol. Ligated plasmid was then transformed into DH5α and plated onto LB agar plates containing 50 μg/mL kanamycin. Individual colonies were picked, grown in the presence of kanamycin, plasmids purified and the ipk gene sequence and frame verified by DNA sequencing (Genewiz).

Expression and Purification of IPK. pET28a-IPK plasmid was transformed into E. coli BL21 (DE3) for protein expression. A single colony was used to inoculate a 3 mL culture in LB media supplemented with 50 μg/mL kanamycin. A 1 L culture containing 50 μg/mL kanamycin in LB media was then inoculated with 1 mL of the overnight culture and grown to an OD₆₀₀ of ˜0.6 at 37° C. with shaking at 300 rpm at which point protein expression was induced by the addition of 1 mM IPTG. The temperature of the incubator-shaker was reduced to 30° C. and the culture incubated for approximately 18 hours. The culture was pelleted at 4000 rpm for 10 mins, the supernatant was decanted, the cell pellet resuspended in 15 mL of lysis buffer (100 mM Tris, 300 mM NaCl, 10% glycerol, pH 8.0) and lysed by sonication. The lysate was then pelleted at 4500 rpm for 10 mins, decanted, and the soluble protein was spun down at 15,000 rpm for 1 hour. The resulting soluble fraction was then purified by fast protein liquid chromatography (FPLC) using nickel-bead column chromatography for the extraction of His₆-tagged proteins. The column was first equilibrated with wash buffer (50 mM TRIS-HCl, 500 mM NaCl, 20 mM imidazole, pH 8.0) prior to loading of the soluble fraction. The soluble fraction was then eluted with elution buffer (50 mM TRIS-HCl, 500 mM NaCl, 200 mM imidazole, pH 8.0) using a gradient of 0% elution buffer 0-7.5 min., 0-50% 7.5-18 min., 50-100% 18-22 min., 100% 22-27.5 min, and equilibrated for additional runs with 0% elution buffer 27.5-35 min. Fractions containing the desired protein were identified by SDS-PAGE and pooled. The pooled protein was then concentrated using a 10 KDa molecular weight cut-off filter (Millipore Amicon-Ultra) and the buffer was exchanged with protein storage buffer (50 mM Tris-HCl, 100 mM NaCl, and 20% glycerol at pH 8.0). Protein aliquots were flash frozen with a dry ice isopropanol bath before storage at −80° C. Protein purity was confirmed by SDS-PAGE while concentration was determined by absorbance using a Pierce Bradford Protein Assay kit.

General Procedure for the Synthesis of Isoprenoid Monophosphates. 400 μmol of the neat alcohol substrate was added to a 15 mL falcon tube. Trichloroacetonitrile (1 mL, 10 mmol) was then added and the mixture was allowed to incubate at room temperature for 5 min. Bis-triethylammonium phosphate (TEAP) solution was prepared by slowly adding solution A (25 mL phosphoric acid, 94 mL acetonitrile) to solution B (110 mL triethylamine, 100 mL acetonitrile) to generate a solution that was 38% solution A and 62% solution B. To the mixture of alcohol and trichloroacetonitrile was added 1 mL of TEAP solution. The mixture was then incubated in a 37° C. water bath for 5 min before another addition of TEAP was added. A total of three additions of TEAP solution were added and incubated. The mixture was then separated by column chromatography using 6:2.5:0.5 iPrOH:conc. NH₄OH:H₂O with silica as the stationary phase. Prior to loading the column, the reaction mixture was diluted 20% v/v with chromatography buffer and the resulting precipitate was pelleted by centrifugation prior to loading of the flash column. Generally, each column was eluted with a total of 400 mL of eluent with a total silica load of 50 mL pre-equilibrated stationary phase slurry. Fractions of 10 mL (around 24 total) were collected after the yellow color of the solvent front disappeared. Fractions were analyzed by using a Shimadzu single quadrupole LCMS-2020 and those containing the diphosphorylated compound, (M−H)−, free of tri- or mono-phosphorylated were pooled. The pooled fractions were then concentrated in vacuo to remove isopropanol and acetonitrile. The concentrated mixture was then filtered using 0.2 m cellulose filter and frozen at −80° C. After being frozen overnight, the sample was lyophilized yielding a salt. The triammonium salt was then characterized and stored frozen as 250 μL 25 mM aliquots.

Mass Spectrometry. Samples from synthesized monophosphates were subjected to negative-mode mass analysis on a Thermo Fisher Scientific Exactive Plus operating with a heated ESI source connected to a UV detector with a Phenomenex Kinetex UPLC C18 column (2.1×50 mm, 2.6 μm particle, 100 Å pores). 1 μL was injected onto a and separated using a series of linear gradients was developed from 20 mM NH₄HCO₃ in H₂O (A) to 4:1 acetonitrile:H₂O (B) at 0.2 mL/min using the following protocol: 0-2 min, 100-80% A; 2-6 min, 80-0% A; 6-7 min, 0% A; 7-7.1 min, 0-100% A; 7.1-12 min, 100% A.

Assay for Initial Activity of Monophosphates with IPK. Fractions from the synthesis of the diphosphates on a 400 μmol scale containing the monophosphates were concentrated in vacuo and resuspended to 5 mg/mL in water. Enzymatic reaction mixtures contained 50 mM Tris (pH 8.0), 2.5 mM MgCl2, 0.05 mM DTT, 1 mM ATP, and 4.2 μg of enzyme in a 200 μL reaction with 40 μL of substrate (1 mg/mL final). A reaction mixture without enzyme was setup as a control. Reactions were incubated overnight at 37° C. and checked by low-resolution LC-MS for diphosphate product formation. Standards from the isolated diphosphates were used to confirm retention time and mass. Reactions with a >10% diphosphate generation as compared to the no enzyme control were selected for kinetic experiments. LC-MS experiments were conducted using a Shimadzu LC-MS 2020 single quadrupole instrument with a Phenomenex Kinetex UPLC C18 column (2.1×50 mm, 2.6 m particle, 100 Å pores) column. 5 μL was injected onto and separated using a series of linear gradients developed from 0.1% formic acid in H₂O (A) to 0.1% formic acid in acetonitrile (B) at 0.2 mL/min using the following protocol: 0-2.2 min, 95-1% A; 2.21-2.6 min, 1% A; 2.61-2.62 min, 1-95% A; 2.63-3.5 min, 95% A. Products of enzymatic reactions were verified by mass and comparison with diphosphate standards previously synthesized.

NADH-Coupled Kinetic Assays. NADH coupled assays were performed with purified enzymes. Reaction progress was monitored by absorbance at 340 nm at 30° C. in a 96-well plate using a Biotek Synergy 4 plate reader (Winooski, Vt.). 200 μL enzymatic mixtures contained 50 mM Tris (pH 8.0), 25 mM KCl, 2.5 mM MgCl2, 0.05 mM DTT, 1 mM ATP, 320 μM NADH, 400 μM phosphoenolpyruvate, 0.5 U pyruvate kinase, 0.7 U lactate dehydrogenase, and various amounts of substrate. Conditions were verified by doubling enzyme and verifying the initial rate was doubled as well.

Kinetics of IPK were done with purified enzyme using 0.09 μg of enzyme per well. Serial dilution was used to generate specific concentrations of substrates (3125, 1562, 781, 625, 391, 195, 98, 24, 12, and 3 μM). Each condition was performed in triplicate. Nonlinear regression was fitted using SigmaPlot (Systat Software Inc., San Jose, Calif., USA).

In silico Modeling of Molecular Properties of Substrates. Compounds were modeled in Chem3D Pro 13.0 (Perkin Elmer, Waltham, Mass., USA). c Log P values were determined by using the c Log P driver. Surface areas were calculated after MM2 minimization using the Connolly Solvent Excluded Volume.

Sequence of Codon-Optimized IPK from Thermoplasma acidophilum:

(SEQ. ID 49)
ATGGATCCGTTCACCATGATGATCCTGAAGATTGGCGGCAGCGTGATTAC
ATAAGAGCGCATATCGCACCGCACGCACCTACGCCATTCGTAGCATCGTG
AACGAGTGCTGAGCGGCATTGAAGATCTGGTGTGCGTGGTGCATGGCGGT
GGTAGCTTTGGCCACATCAAGGCGATGGAGTTTGGTCTGCCGGGTCCGAA
AAATCCGCGTAGCAGCATCGGCTACAGCATCGTGCATCGCGACATGGAAA
ACCTGGACCTGATGGTGATCGACGCAATGATCGAGATGGGTATGCGCCCG
ATTAGCGTGCCGATTAGCGCCCTGCGTTATGATGGTCGCTTTGACTACAC
CCCGCTGATCCGCTATATTGATGCAGGCTTCGTGCCGGTGAGCTATGGCG
ACGTGTATATCAAGGACGAACATAGCTATGGCATCTACAGCGGCGACGAT
ATTATGGCCGATATGGCCGAACTGCTGAAGCCGGATGTGGCCGTGTTCCT
GACCGATGTGGATGGCATCTATAGCAAGGACCCGAAACGCAATCCGGATG
CCGTGCTGCTGCGCGACATCGATACAAACATCACCTTCGATCGCGTGCAG
AACGATGTGACCGGCGGCATTGGCAAGAAATTCGAAAGCATGGTTAAAAT
GAAAAGTAGCGTTAAAAATGGTGTGTACCTGATTAATGGCAATCACCCGG
AGCGCATTGGTGACATCGGCAAGGAGAGCTTCATCGGTACCGTGATTCGC

Compounds

3-Methylbut-2-en-1-yl monophosphate (dimethylallyl monophosphate, DMAP): ¹H NMR (400 MHz, D₂O) δ 5.40 (t, J=5.8 Hz, 1H), 4.34 (dd, J_{H,P}=6.8 Hz, J_{H,H}=6.8 Hz, 2H), 1.75 (s, 3H), 1.70 (s, 3H); ³¹P NMR (162 MHz, D₂O) δ 1.58; HRMS m/z calculated for C₅H₁₁O₄P [M−H⁺]⁻ 165.0322, found: 165.0316.

3-Methylbut-3-en-1-yl monophosphate (isopentenyl monophosphate, IP): ¹H NMR (400 MHz, D₂O) δ 3.92 (dt, J_{H,P}=6.6 Hz, J_{H,H}=6.6 Hz, 2H), 2.34 (t, J=6.6 Hz, 2H), 1.74 (s, 3H); ³¹P NMR (162 MHz, D₂O) δ 1.56; HRMS m/z calculated for C₅H₁₁O₄P [M−H⁺]⁻ 165.0322, found: 165.0317.

But-3-en-1-yl monophosphate (1): ¹H NMR (400 MHz, D₂O) δ 5.70 (ddtd, J=17.0, 9.9, 6.8 Hz, 1H), 4.99 (dd, J=17.0, 3.2 Hz, 1H), 4.97-4.87 (m, 1H), 3.70 (dt, J_{H,P}=6.6 Hz, J_{H,H}=6.6, 2H), 2.20 (dt, J=6.8, 6.6 Hz, 2H); ³¹P NMR (162 MHz, D₂O) δ 2.31; HRMS m/z calculated for C₄H₉O₄P [M−H⁺]⁻ 151.0166, found: 151.0164.

Pent-4-en-2-yl monophosphate (2): ¹H NMR (400 MHz, D₂O) δ 5.87 (ddt, J=17.3, 10.5, 7.1 Hz, 1H), 5.19-5.06 (m, 2H), 4.29 (dtq, J_{H,P}=6.5 Hz, J_{H,H}=6.5, 6.3 Hz, 1H), 2.38-2.32 (m, 2H), 1.24 (d, J=6.3 Hz, 3H); ³¹P NMR (162 MHz, D₂O) δ 1.82; HRMS m/z calculated for C₅H₁₁O₄P [M−H⁺]⁻ 165.0322, found: 165.0320.

3-Bromobut-3-en-1-yl monophosphate (3): ¹H NMR (400 MHz, D₂O) δ 5.81-5.71 (m, 1H), 5.53 (t, J=2.3 Hz, 1H), 3.94 (dt, J_{H,P}=6.4 Hz, J_{H,H}=6.3 Hz, 2H), 2.72 (t, J=6.3, 2H); ³¹P NMR (162 MHz, D₂O) δ 1.59; HRMS m/z calculated for C₄H₈BrO₄P [M−H⁺]⁻ 228.9271; found: 228.9272.

Pent-4-yn-1-yl monophosphate (4): ¹H-NMR (400 MHz, D₂O): δ 3.90 (dt, J_{H,P}=6.4 Hz, J_{H,H}=6.4, 2H), 2.33-2.28 (m, 4H), 1.83-1.80 (m, 1H); ³¹P NMR (162 MHz, D₂O) δ 1.73; HRMS m/z calculated for C₅H₉O₄P [M−H⁺]⁻ 163.01656, found: 163.0164.

2-Methylallyl monophosphate (5): ¹H NMR (400 MHz, D₂O) δ 5.75-5.43 (m, 2H), 4.14 (d, J_{H,P}=6.7 Hz, 2H), 0.72 (s, 3H); ³¹P NMR (162 MHz, D₂O) δ 2.08; HRMS m/z calculated for C₄H₉O₄P [M−H⁺]⁻ 151.0166, found: 151.0162.

Pent-4-en-1-yl monophosphate (6): ¹H-NMR (400 MHz, D₂O): □ 5.91-5.88 (m, 1H), 5.11-4.99 (m, 2H), 3.85 (dt, J_{H,P}=6.6 Hz, J_{H,H}=6.6 Hz, 2H), 2.13 (q, J=6.4 Hz, 2H), 1.70 (p, J=6.6 Hz, 2H); ³¹P NMR (162 MHz, D₂O) δ 2.33; HRMS m/z calculated for C₅H₁₁O₄P [M−H⁺]⁻ 165.03221, found: 165.0320.

(Z)-Hex-2-en-1-yl monophosphate (7): ¹H-NMR (400 MHz, D₂O): □ 5.67-5.61 (m, 2H), 4.41 (dd, J_{H,P}=6.2 Hz, J_{H,H}=8.0 Hz, 2H), 2.10-2.07 (m, 2H), 1.52-1.48 (m, 2H), 0.87 (t, J=6.3 Hz, 3H); ³¹P NMR (162 MHz, D₂O) δ 1.51; HRMS m/z calculated for C₆H₁₃O₄P [M−H⁺]⁻ 179.0479; found: 179.0477.

(E)-2-Hexen-1-yl monophosphate (8): ¹H-NMR (400 MHz, D₂O): □ 5.83-5.80 (m, 1H), 5.66-5.61 (1H, m), 4.29 (dd, J_{H,P}=6.8 Hz, J_{H,H}=6.8 Hz), 2.04 (q, J=7.0 Hz, 2H), 1.40 (q, J=7.5 Hz, 2H), 0.88 (t, J=7.5 Hz, 3H); ³¹P NMR (162 MHz, D₂O) δ 1.92; HRMS m/z calculated for C₆H₁₃O₄P [M−H⁺]⁻ 179.0479, found: 179.0477.

But-3-yn-1-yl monophosphate (9): ¹H-NMR (400 MHz, D₂O): δ 3.91-3.95 (2H, m), 2.52-2.55 (2H, m), 2.36-2.40 (1H, m); ³¹P NMR (162 MHz, D₂O) δ 2.38; HRMS m/z calculated for C₄H₇O₄P [M−H⁺]⁻ 149.0009, found: 149.0007.

TABLE 5
Masses of isolated monophosphates used in initial characterization.

| | Chemical | Calculated Mass | Found |
|---|---|---|---|
| Compound | Formula | [M − H⁺]⁻ | [M − H⁺]⁻ |
| 10 | C₉H₁₁O₄P | 213.0322 | 213.0319 |
| 11 | C₇H₁₃O₄P | 191.0479 | 191.0479 |
| 12 | C₆H₉O₄PS | 206.9886 | 206.9887 |
| 13 | C₅H₇O₅P | 176.9958 | 176.9955 |
| 14 | C₄H₇O₄P | 149.0009 | 149.0008 |
| 15 | C₅H₁₁O₅P | 181.0271 | 181.0271 |
| 16 | C₆H₈NO₄P | 188.0118 | 188.0117 |
| 17 | C₆H₁₁O₄P | 177.0322 | 177.0319 |
| GP | C₁₀H₁₉O₄P | 233.0948 | 233.0944 |
| FP | C₁₅H₂₇O₄P | 301.1574 | 301.1575 |

### Example 4. Remarkable Catalytic and Mechanistic Versatility of a Trans-Prenyltransferase

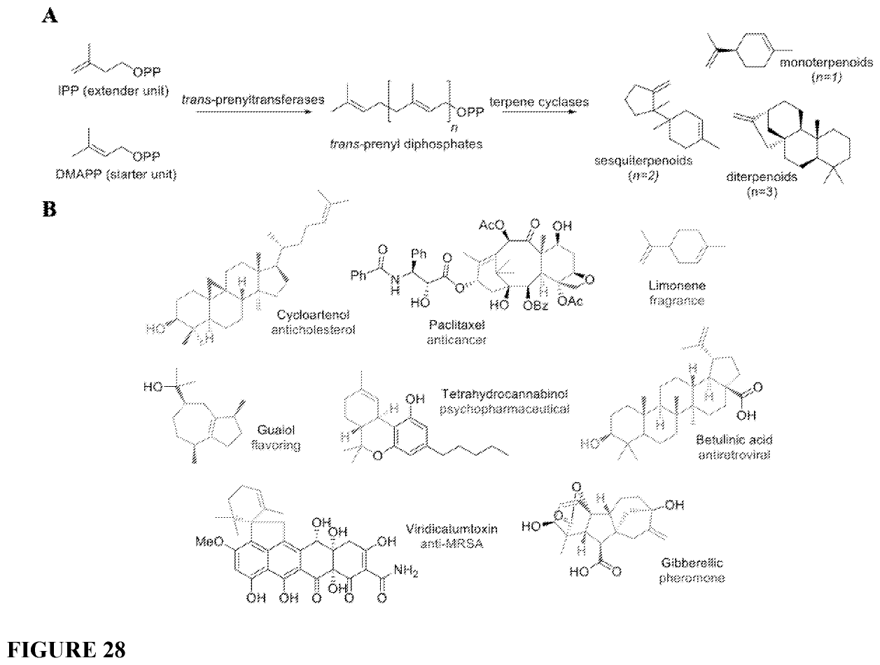

Terpene natural products are used in pharmaceuticals (taxol and artemisinin), pesticides (coumarin and pyrethrin), flavors (hopanoids and menthol), fragrances (citronel and limonene), pigments (carotenoids and xanthophylls), potential biofuels (bisabolane) and a variety of other commercial products. Biosynthesis of terpenes proceeds through condensation of dimethylallyl pyrophosphate (DMAPP) with consecutive isopentenyl pyrophosphate extender units (IPP) to generate prenyl diphosphates. These linear precursors are then cyclized to generate cyclic terpenes via terpene cyclases (FIG. 28, panel A). The utility of terpenes as scaffolds for chemical diversification is limited to leveraging the reactivity of chemical handles that may be present in naturally occurring terpenes. This in turn limits the scope of structure activity relationship (SAR) studies of terpenes. As cyclized terpenes are almost exclusively composed of only hydrocarbon backbones, decoration of these scaffolds is accomplished synthetically by oxidation using C—H activation or biosynthetically using P450s (FIG. 28, panel B). Accordingly, there is emerging interest in harnessing the terpene biosynthetic apparatus to generate non-natural terpene analogues with chemical diversity built into the linear precursors that are in-turn cyclized into these elaborate structures.

In this example, the ability of prenyltransferases to condense non-natural hemiterpenes to form potential linear precursors for terpene cyclases is explored.

Prenyltransferases catalyze elongation of the hemiterpene starter unit, DMAPP, utilizing sequential additions of the hemiterpene extender unit, IPP (Scheme 4). Prenyltransferases are responsible for the generation of linear terpenoid intermediates from these two interconvertible endogenous building blocks and therefore directly impact composition of the final terpene natural product. Prenyltransferases can utilize DMAPP analogues containing alternative diphosphate moieties, alkyl extensions at the methyl positions, epoxidized alkenes, and unsaturated alkenes. Even though a range of DMAPP analogues can potentially serve as substrates for prenyltransferases, the structural diversity of known analogues is limited to derivatives of allylic diphosphates containing trisubstituted alkenes. While extensive studies on DMAPP derivatives have been conducted, minimal work has been carried out to describe the promiscuity of prenyltransferases with IPP analogues. Work done previously with IPP analogues has been limited to extender units containing the natural homoallylic diphosphate core with no exploration into alternative nucleophiles and only a single study using one substrate altering the spacing between the nucleophile and diphosphate moiety. To overcome this severely limited scope, it was hypothesized that prenyltransferases might be able to use a variety of nucleophiles in place of the natural extender unit IPP as a general strategy to biosynthesize isoprenoid analogues. In support of this, prenyltransferases catalyze carbon-carbon bond formation simply by directing the nucleophilic attack of a relatively non-nucleophilic homoallylic alkene to an allylic carbocation followed by stereospecific desaturation. Importantly, additional diversity accessed through this approach could provide new chemical handles or varying oxidation states of carbons in terpene backbones not afforded by endogenous P450s.

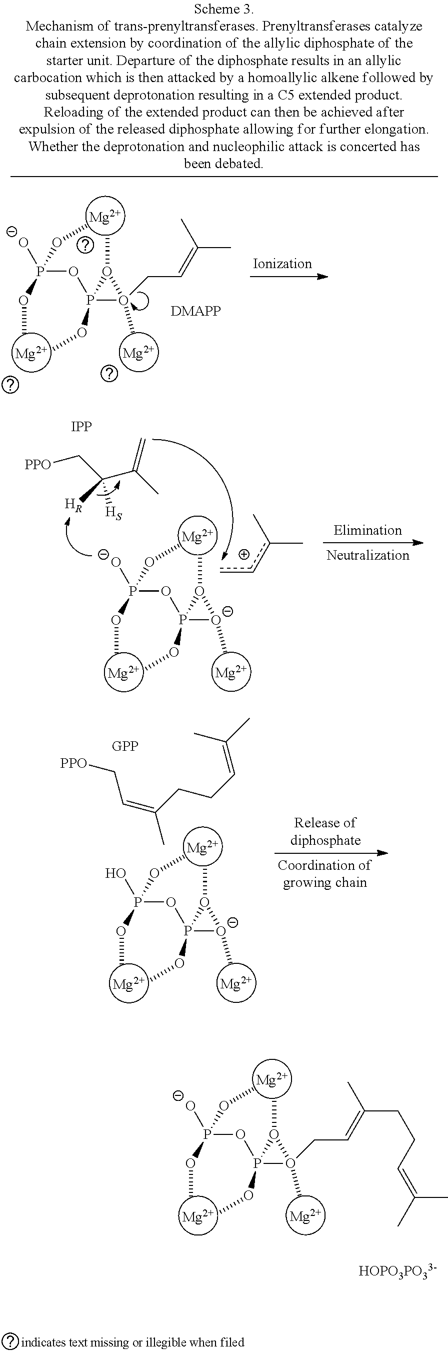

As the substrate scope of prenyltransferases has not been found to be limited, the tolerance of prenyltransferases towards unnatural nucleophiles should be further characterized for their ability to catalyze irreversible carbon-carbon bond formation. To more fully understand the inherent promiscuity of prenyltransferases, IspA, a farnesyl diphosphate synthase (FPPase) from Escherichia coli, was characterized for its ability to utilize a panel of unnatural extender units for the generation of extended prenyl diphosphate analogues.

Results and Discussion

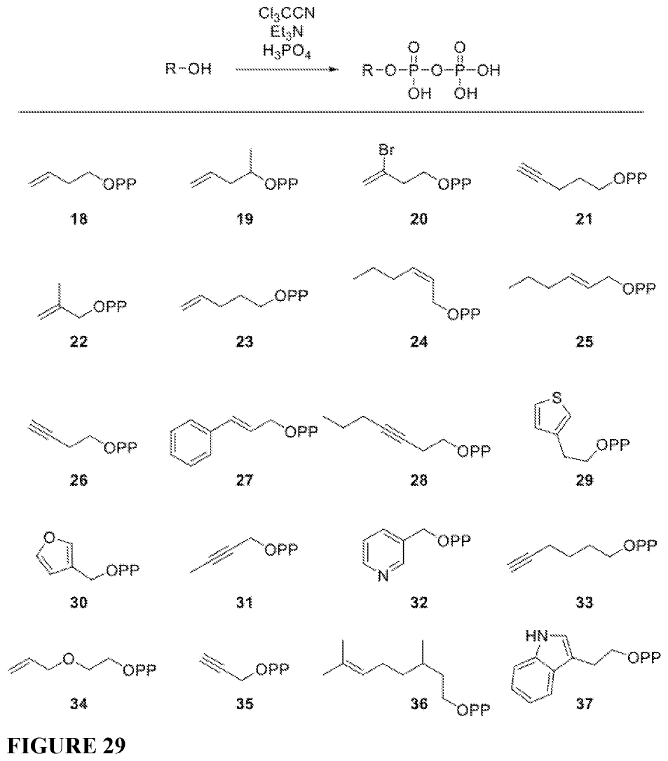

Design and Synthesis of a Panel of DMAPP/IPP Analogues. A panel of potential extender and starter units was synthesized from their respective commercially available alcohols, as outlined in Example 3. A wide panel of alcohols was selected for phosphorylation and included functionalities such as alkynes, aromatics, and hetereoatomic moieties as potential nucleophiles in chain extension (FIG. 29).

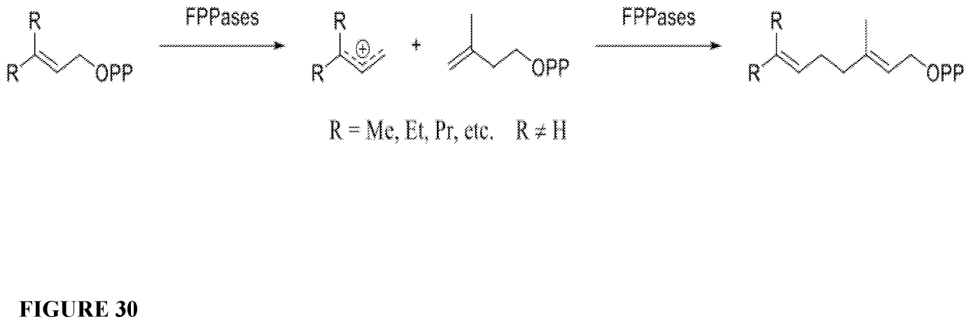

IspA Specificity with Starter Unit Analogues. The majority of research on prenyltransferase substrate tolerance has been focused on varying DMAPP. While the substrate scope of these enzymes has not been fully characterized, all substrates previously tested by others in place of DMAPP have had structural similarity (FIG. 30). Here, the panel members (FIG. 29) are incredibly diverse. Each unnatural diphosphate 18-37 was first tested as a replacement to DMAPP as foreign starter units in the presence of the natural extender unit IPP.

Prenyltransferase reactions were run with 200 μM starter unit and 600 μM extender unit in 50 mM Tris and 10 mM MgCl2 at pH 7.5 with enzyme. Reactions were allowed to proceed overnight at 37° C. before being quenched with an equal volume of methanol. Products were analyzed by high-res mass spectrometry.

None of the substrates resulted in the detection of the predicted condensation product, as judged by LC-MS analysis of the product mixtures. Accordingly, none of the diphosphates are able to act as unnatural starter units, most likely because they lack the trisubstituted allylic double bond featured in previously reported DMAPP analogues utilized. Only analogues 27, 30, and 32 included trisubstituted allylic double bonds, however these analogues may have been too sterically demanding to be accommodated in the active site of IspA.

Use of Extender Unit Analogues. As IPP recognition is presumably principally driven by the electrostatic forces of the diphosphate rather than that of the greasy interactions of the alkyl tail, it was envisioned that the enzyme may be agnostic towards the remaining part of the extender unit. Besides the pyrophosphate segment, IPP must sterically fit into the extender unit binding pocket and must be recognized by low energy lipophilic binding forces facilitated by the displacement of water. The homoallylic alkene of IPP is unactivated suggesting that the FPPases may also accommodate alternative 71-bonds as nucleophiles. While studies have shown that farnesyl pyrophosphate synthases (FPPases) are quite promiscuous towards DMAPP analogues, only a few reports provide evidence for the incorporation of IPP analogues which have consisted of aliphatic analogues and a chlorinated analogue, all of which contain the seemingly requisite homoallylic diphosphate.

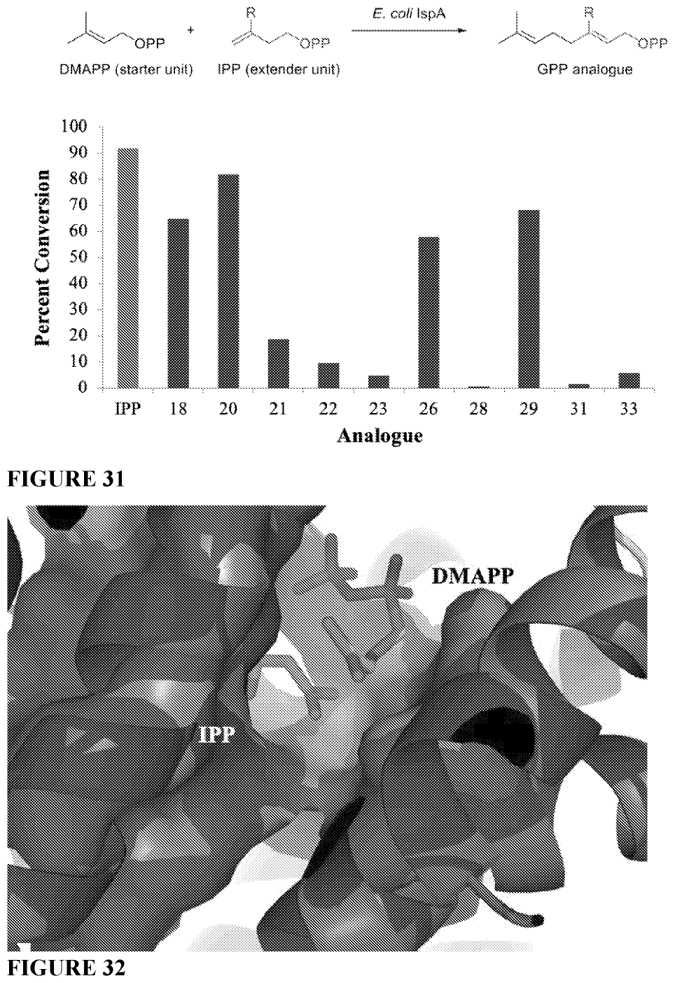

Next, the panel members were tested as potential replacements of IPP (unnatural extender units) by incubating IspA with 18-37 and DMAPP as the usual starter unit. Remarkably, most of the diphosphate extender units (18, 20-23, 26, 28-29, 31, 33) were used by IspA to generate the corresponding geranyl pyrophosphate (GPP) analogues, as judged by LC-MS analysis of the product mixtures (FIG. 31). In agreement with findings about chlorinated-IPP analogue incorporation, once a prenyltransferase has catalyzed an unnatural chain-extension with an unnatural extender unit, it will not subsequently use the GPP analogue as a substrate for consecutive condensations. No product ions consistent with multiple unnatural extensions (corresponding C15 products) could be detected.

Alkene diphosphate 18 was efficiently utilized by IspA to extend the natural starter unit DMAPP. Compared to the natural extender unit IPP, 18 only lacks a methyl group, and it is expected to occupy the IspA extender unit binding pocket as well as IPP does. Notably, the vinyl bromide 20 was used almost as efficiently as IPP. Low activity of IspA towards 22 is of note as extender units do not typically have allylic diphosphates unless the prenyltransferase is catalyzing head-to-head condensation. The alkene 23 was also found to be a good substrate indicating that IspA was able to accommodate catalysis of carbon-carbon forming reactions between unactivated 71-systems and DMAPP as is the case with the use of the natural substrate IPP (FIG. 32).

As there is ample space for IPP analogues to occupy the active site in the presence of DMAPP, it is not entirely unexpected that a properly positioned nucleophile (e.g., 18, 20-23) can participate in catalysis (FIG. 31). Notably, the two highest molecular weight analogues were not utilized by IspA (e.g., 27 and 37). In addition, analogues with improperly positioned nucleophiles did not facilitate carbon-carbon bond formation either (24, 25, 27). For example, π-bonds extending too short towards the presumed allylic carbocation of the ionized starter unit (22 and 31) and those extending past (21 and 33) were poor substrates for carbon-carbon bond formation, as compared to those analogues with appropriately positioned nucleophiles. To characterize this kinetically, the commercial PiPer assay was used (measure pyrophosphate release), however, no correlation between carbon-carbon bond formation and the rate of pyrophosphate was observed indicating pyrophosphate release could not be used to determine kinetic parameters of these reactions. This could be due to the release of pyrophosphate from DMAPP when triggered by the binding of an analogue that acts as an inhibitor rather than a substrate.

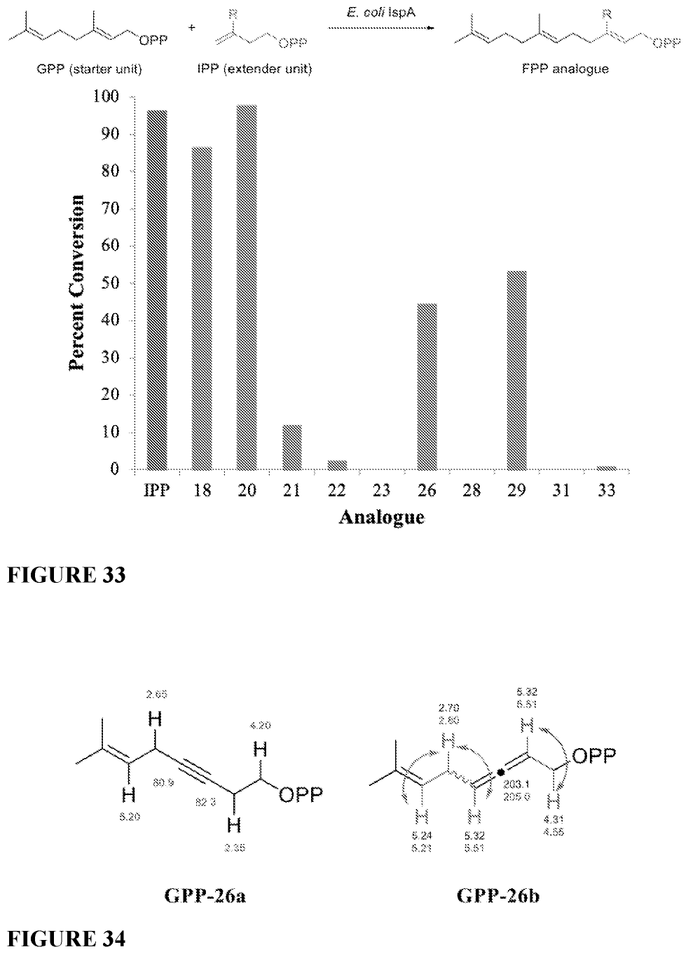

Cumulatively, this data suggests that simple sterics and nucleophile positioning are sufficient to predict the substrate promiscuity of IspA. If this is accurate, then the extender unit specificity when an alternative, longer starter unit is used, should largely parallel that with DMAPP. To test this hypothesis, diphosphates 18-37 were each incubated with IspA in the presence of GPP as a starter unit and production of the corresponding farnesyl pyrophosphate (FPP) analogues was determined by LC-MS analysis of the product mixtures (FIG. 33).

Most of the IPP analogues were efficiently utilized by IspA with GPP as the starter unit. For example, conversion of 26 and 29 were almost as good as that with IPP. Almost quantitative conversion was detected with 18 and 20. The only analogues that were not detectable substrates were 23, 28, and 31. While the specificity of analogue use in place of IPP is not identical with DMAPP and GPP as starter units, the similarity is striking. This implies that the binding pocket in which the starter unit occupies is entirely separate of that in which the extender unit resides.

In addition to the analogues discussed so far, utilization of 21, 23, 26, 28, 29, 31, and 33 reveal that IspA displays unprecedented use of unnatural nucleophiles, including non-homoallylic alkenic (23), alkynyl (21, 26, 28, 31, and 33), and aromatic π electrons (29). While the aromatic analogues 27, 30, 32, and 37 did not serve as an extender unit, 29 was accepted by IspA with both DMAPP and GPP as the starter unit. It is likely that the electron-rich thiophene motif increases the nucleophilicity of the aromatic π-electrons.

Unprecedented Use of Alkynes as Nucleophiles by IspA. Of particular interest were the alkynic analogues utilized by IspA, 21, 26, 28, 31, 33. If the alkynic analogues were activated in the same manner as the natural substrate and assuming the same stepwise mechanism was utilized, the 7E electrons of the alkyne would behave as a nucleophile and would subsequently generate an alkenyl cation before desaturation (Scheme 4). In the case of a terminal alkyne as the extender unit, desaturation by abstraction of Hₐ or H_{b} would generate the corresponding internal alkyne or allene, respectively. For the internal alkyne as the extender unit (28 and 31), only desaturation by abstraction of Hₐ is possible, forming the allene as the only possible product. While mechanistically indicative of an allene, 28 and 31 were very poor substrates (<5% conversion) and as such were not considered for scale-up and isolation.

The generation of an allene via prenyltransferase catalysis, regardless of substrate identity, is unprecedented and prompts multiple mechanistic considerations. First, if ionization of the starter unit precedes attack of the terminal alkyne on the carbocation, it is possible that the resulting leaving group, pyrophosphate, can act as a base and generate the zwitterionic intermediate DMAPP and 26a (Scheme 4, Route A). While this is possible, the presence of a zwitterionic species in an active site is unlikely. Another possibility is the attack of the terminal alkyne to generate a vicinal carbocation (26b) as is seen in the addition of HCl across alkynes (Route B). A third possibility would be an E2′/S_{N}2′ mechanism in which little carbocationic character of the extended species is achieved due to the concerted nature of addition and elimination analogous to the generation of hopene from squalene by hopanoids cyclases in triterpene biosynthesis (Route C). Several lines of experimental evidence support the generation of allenes via IspA catalysis.

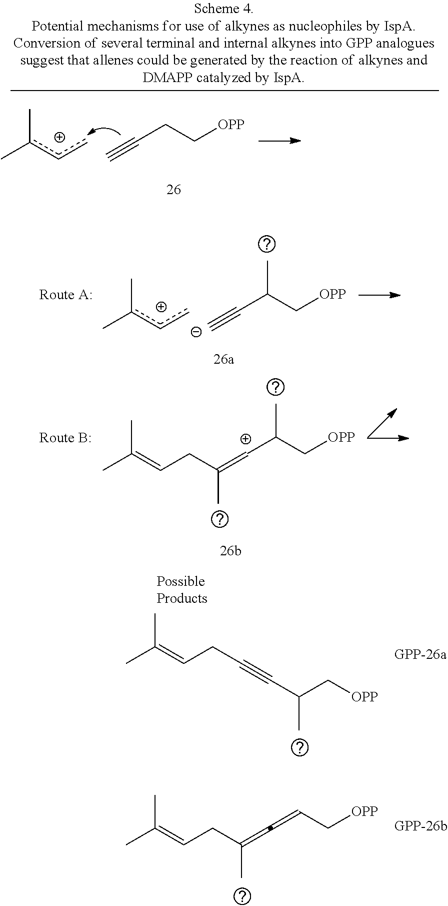

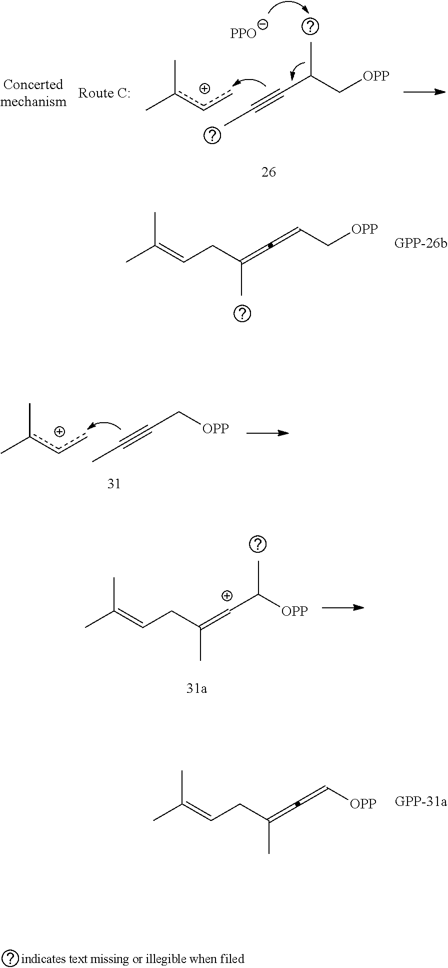

First, the internal alkynes (28 and 31) were utilized by IspA, albeit very poorly with DMAPP as the starter unit, and the allene must be generated as only a single proton is available (31a) to regenerate a neutral species (Scheme 4).

To fully elucidate this elusive structure, the enzymatic reaction using 26 and DMAPP was scaled-up for product isolation. Removal of the protein followed by chromatography provided a product with the expected mass for the allene GPP-26b. For comparison, the hypothetical alkyne GPP-26a was chemically synthesized.

The ¹³C NMR spectrum of the isolated product included a signal at 206 ppm characteristic of a carbon at the center of an allene. At the same time, signals characteristic of an internal alkyne (75-85 ppm) were not observed. Moreover, the ¹H, ¹³C, and ¹H-¹H COSY NMR spectra were in full agreement with the assigned structure of GPP-26b (FIG. 34). Finally, an optical rotation measurement provided evidence of enantiomeric excess. This observation is fully consistent with the expected stereospecific course of the enzyme catalyzed reaction versus the non-enzymatically generated allene from isomerization, which would be racemic. Thus, consistent with the accepted mechanism of IspA-catalyzed chain extension, enantiomeric excess is enabled by stereospecific deprotonation of the prochiral hydrogen vicinal to the carbocation generated from nucleophilic attack by π-electrons of the extender unit. While insight into the mechanism through Routes B or Route C (Scheme 4) have not been deciphered, generation of a vicinal carbocation is highly unlikely which suggests that use of alkynyl nucleophiles proceeds through a concerted mechanism in opposition to that of the natural reaction.

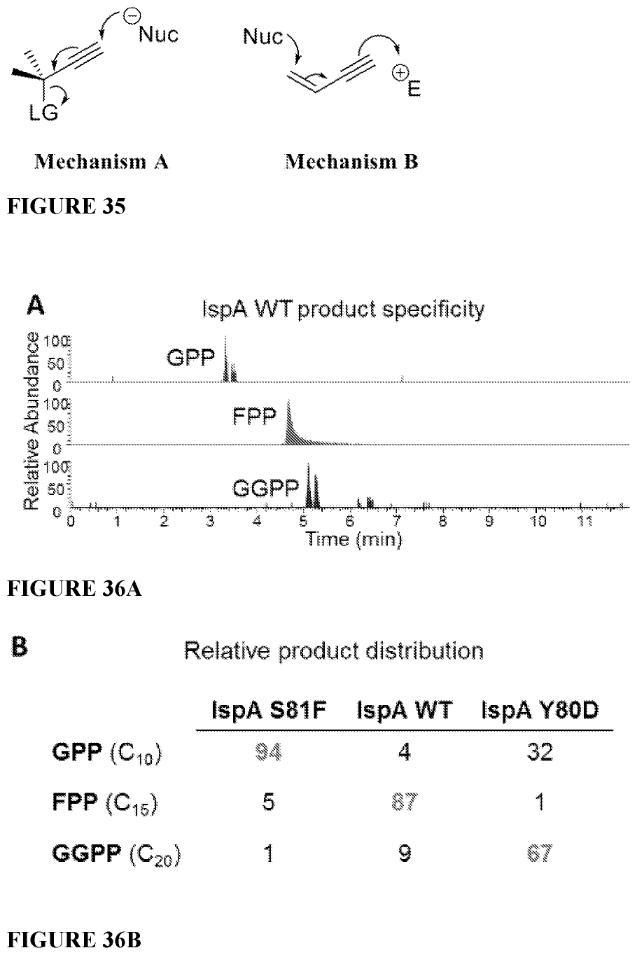

To summarize, all the available evidence is fully consistent with the IspA-catalyzed formation of an allene from alkynyl IPP substrate analogues. Allenes are usually biosynthesized through enzyme catalyzed isomerization of alkyltriynes or base catalyzed elimination of hydrogen peroxide from allyl peroxides. This proposed manner of generating allenes from the addition of an electrophile is unprecedented in both biosynthesis and organic chemistry (FIG. 35).

While there are many methods for stereospecific generations of allenes, the most common is probably through a S_{N}2′ mechanism. For instance, in the chemical synthesis of panacene has used stereospecific anti-S_{N}2′ addition on a propargylic mesylate with LiCuBr₂. This mechanism is often employed with the use of Gilman reagents and propargylic leaving groups. The mechanism utilized by IspA for generation of an allene using an alkyne-containing extender unit is most like mechanism B. This mechanism is usually employed with an intramolecular nucleophile. When trying to synthesize the allene in panacene, Feldman attempted addition of a halogen using NBS, however this reaction did not occur stereospecifically. While most similar to mechanism B, use of alkynes by IspA presumably occurs stereospecifically due to steric constraints provided by the active site of the enzyme. Mechanism B utilizes the addition of a nucleophile to a conjugated alkene to facilitate a second addition to an electrophile while the biosynthesis of GPP-26b most likely is Brønsted base catalyzed addition of an alkyne to an electrophile in a stereospecific manner. Optical rotation of the isolated allene GPP-26b suggests this is the case.

Substrate Specificity of IspA “Chain Extension” Mutants. Beyond understanding the substrate scope and mechanism of IspA, this enzyme and other prenyltransferases have been the subject of multiple engineering efforts to better understand and manipulate carbon-carbon bond formation using the natural substrates. Perhaps most notably, enzyme engineering efforts afforded by chimeric shuffling have resulted in biocatalysts capable of all four reaction modes available to prenyltransferases: head-to-tail, head-to-head, and head-to-middle.

In addition to forming various types of structures, these enzymes are also capable of catalyzing multiple extension events. For instance, IspA catalyzes two extensions of DMAPP with IPP to first generate geranyl pyrophosphate (GPP) and then farnesyl pyrophosphate (FPP). Using random mutagenesis, the product specificity of IspA has been shifted by discovering mutants capable of a third extension to generate geranylgeranyl diphosphate (GGPP) or limit IspA to a single extension to generate GPP. These mutations were made using site-directed mutagenesis and their activities were confirmed in vitro (FIGS. 36A-36B).

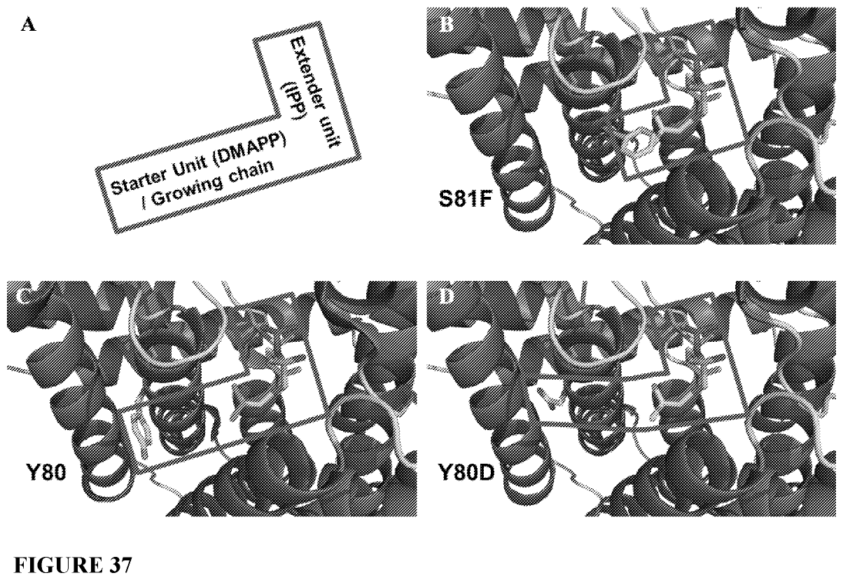

These mutations were mapped onto an IspA crystal structure and map to the bottom of a cavity in the active site where the prenyl acceptors, DMAPP and GPP, extend. The mutation S81F appears to prematurely block the active site to only allow the binding of DMAPP, but not the extended product GPP, effectively limiting IspA to a single extension event. In contrast, Tyr80 is a residue that appears to limit the number of additional extensions to produce isoprenoids no longer than GPP. The mutation Y80D introduces a shorter amino acid sidechain that appears to point away from the active site, allowing for binding of FPP and the subsequent production of GGPP. These mutations therefore act as a molecular ruler (FIG. 37).

While this molecular ruler hypothesis has been confirmed in vitro with the natural substrates, we wanted to see if these mutations had any effect on the ability of IspA to use unnatural extender units. Accordingly, the mutations S81F and Y80D were each introduced into the wild-type IspA gene sequence by site-directed mutagenesis. The mutant proteins were expressed in E. coli and purified as hexa-histidine fusion proteins via immobilized metal affinity chromatography. Next, the wild-type, S81F and Y80D IspA were each incubated with IPP and each of 18-37 and the resulting product mixtures analyzed by LC-MS analysis in order to quantify the total conversion of starter unit into C10 (GPP) products. As was observed before with the wild-type IspA, product could not be detected when any of the non-natural pyrophosphates were used in the place of DMAPP as the starter unit with the IspA mutants S81F and Y80D. Thus, consistent with the hypothesized ‘molecular ruler’ role of these two positions, these two mutations are not able to impact utilization of non-natural starter units in this context.

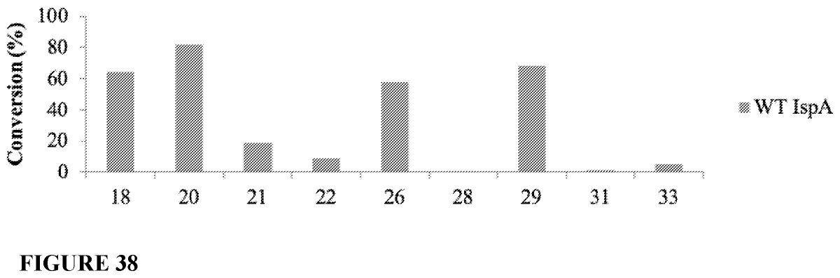

Notably, when the mutants were tested with the unnatural extender units in place of IPP, the specificity of the mutants appeared quite similar to that of the wild-type enzyme (FIG. 38). For example, pyrophosphates 18, 20, 26, and 29 were the most efficient analogues, as judged by percent conversion, by the mutant and wild-type enzymes. Similarly, 28 was converted to product very poorly for all three enzymes. Conversion of 21, 22, 31, 33

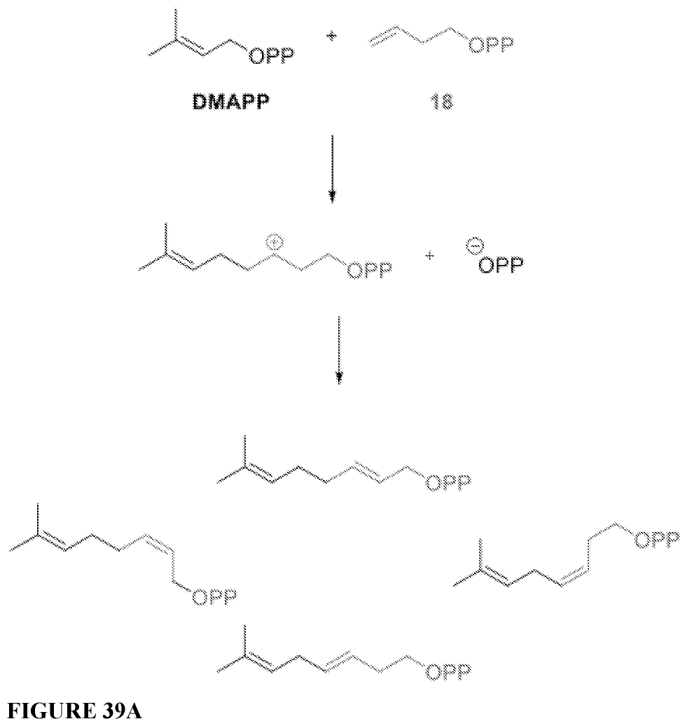

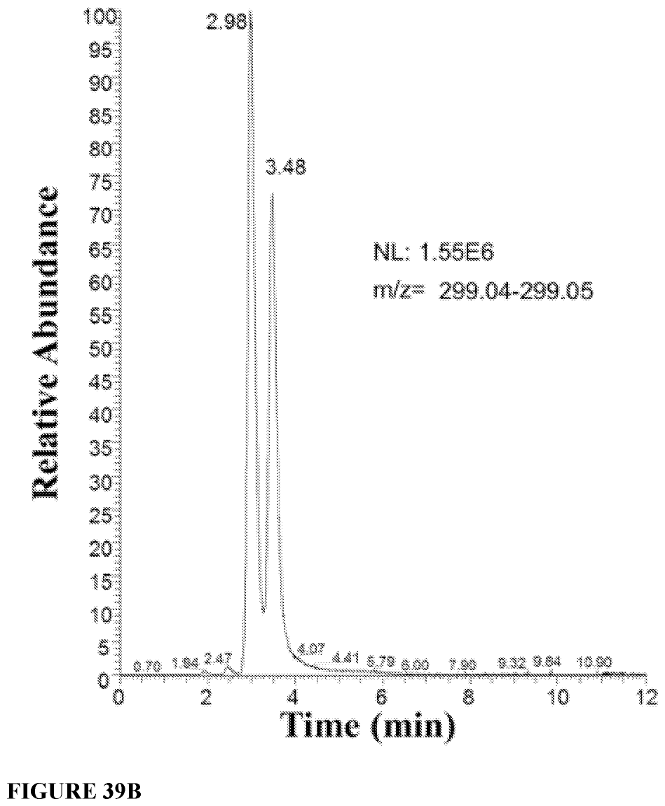

to product could not be detected with IspA S81F and Y80D, likely because the mutants are less active than the wild-type with all substrates, and the level of activity with these analogues fall below the detection limit of the LC-MS assay. It is well established that wild-type IspA produces a single GPP (C10) product with the natural substrates, DMAPP and IPP, highlighting the strict stereo- and regio-specificity of proton abstraction from the intermediate. Notably however, LC-MS analysis of the product profile generated by the wild-type, Y80D, and S81F IspA using some of novel non-natural extender units (18 and 22) revealed several product ions with unique retention times. For example, using 18 as the non-natural IPP analogue, two unique ions with masses consistent with the predicted C10 GPP analogue were detected (FIGS. 39A-39B). Each product ion could represent the corresponding cis/trans isomer or an isomer generated via carbocation rearrangement. While the enzyme catalyzed additions of the analogues to DMAPP sometimes produced multiple products, these product ratios seem to be consistent among the molecular ruler mutations. The hypothesis of these residues acting as molecular rulers and having no impact on catalysis beyond distinction of starter units holds with the use of analogues tested in this example.

Conclusions and Future Work

Without engineering IspA, the wild-type enzyme was able to catalyze chain extension using 10 of 20 unnatural extender units containing various functionalities. While some diversification of prenyl units has been achieved using unnatural prenyl diphosphates, this unprecedented use of non-natural extender units emphasizes not only the substrate promiscuity of FPPases, but the mechanistic plasticity. Use of aromatic- and alkynyl-n systems as nucleophiles suggests these enzymes are capable of far more than addition of an alkene to an allylic diphosphate.

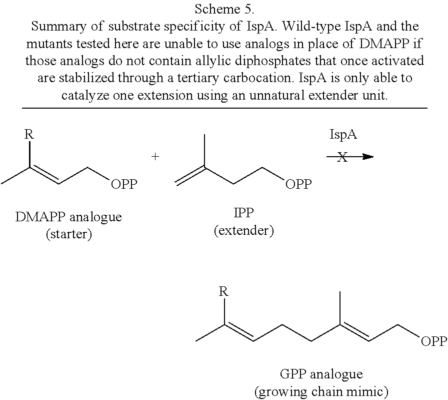

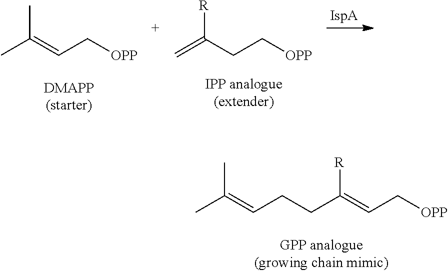

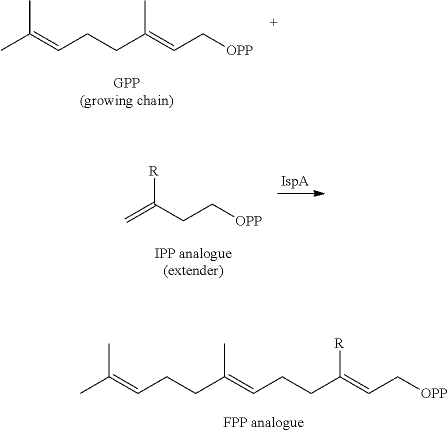

As engineering of chain elongating prenyltransferases has been accomplished in vivo by screening using volatiles or colorimetric methods, it is expected that the same could be done with these analogues. Products generated by terpene cyclases could generate chiral quaternary carbons replacing the natural non-chiral gem-dimethyl moieties. If the terpene cyclases naturally generate alkenic methyls, proposed substitutions with the brominated substrate could afford unnatural terpenes with sp² halogens enabling Heck coupling.

Diversification of prenyl units and by extension, generation of GPP and FPP analogues, may provide unprecedented diversity in cyclized terpenes. As terpene cyclases have been shown to be quite promiscuous towards unnatural analogues, these analogues may provide further diversity in the extensive collection of cyclized terpenes afforded by the elaborate and highly coordinated cyclization of linear precursors. As chemical alteration of this expansive natural product class is limited towards leveraging existing oxidation or providing oxidation through C—H activation, the diversity afforded by this approach could complement synthetic efforts towards alteration of such complex ring systems. By directly incorporating oxidation and diversity into the backbone of these structures, chemists may be afforded with built-in chemical handles or even new structures not provided by Nature due to the potential of novel ring structures generated from substrate directed cyclization patterns instead of strictly enzyme guided bond formation.

Methods

General. All plasmids were verified by DNA sequencing. Purifications of all DNA were performed with kits from BioBasic. Synthetic oligonucleotides were purchased from IDT (Coralville, Iowa, USA). All plate reader assays were performed using a BioTek Hybrid Synergy 4 plate reader (Winooski, Vt., USA). Restriction enzymes were purchased from New England Biolabs (Ipswich, Mass., USA). Polymerase chain reactions were conducted using Phire Hot Start II DNA Polymerase from ThermoFisher Scientific (Waltham, Mass., USA). Chemicals were purchased from Sigma Aldrich (St. Louis, Mo., USA) and Alfa Aesar (Haverhill, Mass., USA).

Gene Cloning. IspA (NP_414955.1) was PCR amplified from E. coli BL21 genomic DNA using the oligos IspA-BamHI-FWD and IspA-XhoI-REV, and cloned into pET28a using BamHI and XhoI restriction sites. PCR was performed using Phire Hot Start II polymerase (ThermoScientific) according to the manufacturer's protocol. PCR product was purified prior to and after digestion by agarose gel electrophoresis. Digested PCR product and similarly treated pET28a were ligated at room temperature with T4 ligase (New England BioLabs) according to supplier's protocol. Ligated plasmid was then transformed into DH5α and plated onto LB agar plates containing 50 μg/mL kanamycin. Individual colonies were picked, grown in the presence of kanamycin, plasmids purified and the IspA gene sequence verified by DNA sequencing.

Site-Directed Mutagenesis of IspA. The mutations S81F and Y80D were introduced into the wild-type IspA template by QuickChange II site-directed mutagenesis. Primers used previously were employed¹⁰⁶. Briefly, mutagenic primers for each mutation were used to amplify the IspA gene from the pET28a-IspA template using Pfu turbo polymerase, digested with DpnI to remove the parent template, and ligated using T4 DNA ligase. Ligation mixtures were then transformed into E. coli DH5α and plated onto LB agar plates containing 50 μg/mL kanamycin. Individual colonies were then used to prepare plasmids, and the desired mutations confirmed by sequencing. No spurious mutations were identified. Purified plasmid was transformed into E. coli BL21 DE3 for expression.

Expression and Purification of IspA. Each pET28a-IspA, pET28a-IspA-S81F, and pET28a-IspA-Y80D plasmid was transformed into E. coli BL21 (DE3) for protein expression. A single colony was used to inoculate a 3 mL culture in LB media supplemented with 50 μg/mL kanamycin. A 1 L culture containing 50 μg/mL kanamycin in LB media was then inoculated with 1 mL of the overnight culture and grown to an OD600 of ˜0.6 at 37° C. with shaking at 300 rpm at which point protein expression was induced by the addition of 1 mM IPTG. The temperature of the incubator-shaker was reduced to 18° C. and the culture incubated for approximately 18 hours. The culture was pelleted at 4000 rpm for 10 mins, the supernatant was decanted, the cell pellet resuspended in 15 mL of lysis buffer (100 mM TRIS-HCl, 300 mM NaCl, 10% glycerol, pH 8.0) and lysed by sonication. The lysate was then pelleted at 4500 rpm for 10 mins, decanted, and the soluble protein was spun down at 15,000 rpm for 1 hour. The resulting soluble fraction was then purified by fast protein liquid chromatography (FPLC) using nickel-bead column chromatography for the extraction of His6-tagged proteins. The column was first equilibrated with wash buffer (50 mM TRIS-HCl, 500 mM NaCl, 20 mM imidazole, pH 8.0) prior to loading of the soluble fraction. The soluble fraction was then eluted with elution buffer (50 mM TRIS-HCl, 500 mM NaCl, 200 mM imidazole, pH 8.0) using a gradient of 0% elution buffer 0-7.5 min., 0-50% 7.5-18 min., 50-100% 18-22 min., 100% 22-27.5 min, and equilibrated for additional runs with 0% elution buffer 27.5-35 min. Fractions containing the desired protein were identified by SDS-PAGE and pooled. The pooled protein was then concentrated using a 10,000 molecular weight cut-off filter (Millipore Amicon-Ultra) and the buffer was exchanged with protein storage buffer (50 mM TRIS-HCl, 100 mM NaCl, and 20% glycerol at pH 8.0). Protein aliquots were flash frozen with a dry ice ethanol bath before storage at −80° C. Protein purity was confirmed by SDS-PAGE while concentration was determined by absorbance using a Pierce Bradford Protein Assay kit.

In silico Modeling of IspA Mutants. IspA mutants were modeled using PDB file 1RQI with PyMol. PyMol's mutagenesis wizard tool was used for visualization of different chain length determinant mutants. Modeling of analogues with IspA was conducted using Glide (Schrödinger) with DMAPP docked.

General Procedure for the Synthesis of Isoprenoid Diphosphates. 4000 μmol of the neat alcohol substrate was added to a 50 mL polypropylene tube. Alcohols were purchased from Sigma Aldrich. 7-Methyloct-6-en-3-yn-1-ol was synthesized using a method adapted from Brunel, Y. and Rousseau, G. J. Org. Chem., 1996, 61 (17), pp 5793-5800. Trichloroacetonitrile (10 mL) was then added and the mixture was allowed to incubate at room temperature for 5 min. Bis-triethylammonium phosphate (TEAP) solution was prepared by slowly adding solution A (25 mL phosphoric acid, 94 mL acetonitrile) to solution B (110 mL triethylamine, 100 mL acetonitrile) to generate a solution that was 38% solution A and 62% solution B. To the mixture of alcohol and trichloroacetonitrile was added 10 mL of TEAP solution. The mixture was then incubated in a 37° C. water bath for 5 min before another addition of TEAP was added. A total of three additions of TEAP solution were added and incubated. The mixture was then separated by column chromatography using 6:2.5:0.5 iPrOH:conc. NH₄OH:H₂O with silica as the stationary phase. Prior to loading the column, the reaction mixture was diluted 20% v/v with chromatography buffer and the resulting precipitate was pelleted by centrifugation prior to loading of the flash column. Fractions were analyzed by using a Shimadzu single quadrupole LCMS-2020 and those containing the diphosphorylated compound, (M−H)−, free of tri- or mono-phosphorylated were pooled. The pooled fractions were then concentrated in vacuo to remove isopropanol and acetonitrile. The concentrated mixture was then filtered using 0.2 m cellulose filter and frozen at −80° C. After being frozen overnight, the sample was lyophilized yielding a salt. The triammonium salt was then characterized and stored frozen as 250 μL 25 mM aliquots.

Mass Spectrometry. Samples were subjected to negative-mode mass analysis on a Thermo Fisher Scientific Exactive Plus operating with a heated ESI source in the negative mode connected to a UV detector with a Phenomenex Kinetex UPLC C18 column (2.1×50 mm, 2.6 μm particle, 100 Å pores). 1 μL was injected onto a and separated using a series of linear gradients was developed from 20 mM NH₄HCO₃ in H₂O (A) to 4:1 acetonitrile:H₂O (B) at 0.2 mL/min using the following protocol: 0-2 min, 100-80% A; 2-6 min, 80-0% A; 6-7 min, 0% A; 7-7.1 min, 0-100% A; 7.1-12 min, 100% A. Linear detection ranges for GPP and FPP were determined by serial dilution and were found to be from 5 μM to 500 μM.

Phenyltransferase Assays. 1 μL IspA WT, IspA S81F, or IspA Y80D (4.24, 3.13, and 5.78 μg/L respectively) An aliquot (5 μL) of wild-type or mutant IspA was added to a total volume of 100 μL 50 mM Tris buffer containing 200 μM dimethylallyl diphosphate (starter unit), 600 μM isopentenyl diphosphate (extender unit), and 5 mM MgCl2 at pH 7.5 and incubated at 37° C. Analogue reactions were conducted in the same manner. Reactions were initiated by the addition of purified enzyme and were incubated overnight. Aliquots were quenched after 16 hours with twice the volume of methanol and stored at −20° C. until analysis. Conversions were internally quantified by dividing the extracted ion counts (EIC) of the products by the EIC of DMAPP plus EIC of the product and multiplying the resulting fraction by 100.

Isolation of Prenyltransferase Product. The prenyltransferase reactions were scaled to 50 mL and were carried out in 50 mL polypropylene tubes with incubation at 37° C. in a shaker at 250 rpm. Importantly, agitation and introduction of ambient air resulted in a loss of product formation potentially attributable to oxidation of IspA. Reactions were run for 48 hours and monitored by LC-MS. Upon complete consumption of DMAPP, Chelex (200 mg) was added to the mixture and the reaction was incubated as before for 3 hours in order to remove Mg²⁺. The resin was then pelleted by centrifugation and the reaction was then passed through a 3K MWCO filter (Millipore) to remove protein. The mixture was then lyophilized to yield a white precipitate. The mixture was then suspended in 10 mL of 0.1 M ammonium bicarbonate.

Semipreparative HPLC was carried out using a Phenomenex Kinetex HPLC C18 column (10×150 mm, 2.6 μm particle, 100 Å pores) with a gradient consisting of an aqueous mobile phase of 25 mM ammonium bicarbonate (A) and an organic mobile phase of acetonitrile (B) at 4.5 mL/min using the following protocol: 0-5 min, 100% A; 5-30 min, 100-60% A; 30-35 min, 0% A; 35-40 min, 100% A. Fractions were collected using a fraction collector with 60 second windows. Fractions containing the desired product were identified by LC-MS, pooled, and lyophilized.

PᵢPer™ Assay. The PiPer assay (Invitrogen) was carried out according to manufacturer's instructions. Reactions were carried out as outlined in Prenyltransferase Assays.

Compounds in this Study

3-Methylbut-2-en-1-yl diphosphate (dimethylallyl diphosphate, DMAPP): ¹H NMR (400 MHz, D₂O) δ 5.43 (t, J=7.0 Hz, 1H), 4.43 (dd, J_{H,P}=7.0 Hz, J_{H,H}=7.0 Hz, 2H), 1.76 (s, 3H), 1.71 (s, 3H); ¹³C NMR (101 MHz, D₂O) δ 140.1, 119.7 (d, J_{C,P}=8.2 Hz), 62.7 (d, J_{C,P}=5.4 Hz), 25.0, 17.3; ³¹P NMR (162 MHz, D₂O) δ −6.04 (d, J=21.7 Hz), −9.38 (d, J=21.6 Hz); HRMS m/z calculated for C₅H₁₂O₇P₂ [M−H⁺]⁻ 244.9985, found: 244.9986.

3-Methylbut-3-en-1-yl diphosphate (isopentenyl diphosphate, IPP): ¹H NMR (400 MHz, D₂O) δ 4.02-3.92 (m, 2H), 2.30 (t, J=6.7 Hz, 2H), 1.68 (s, 3H); ¹³C NMR (101 MHz, D₂O) δ 143.8, 111.6, 64.1 (d, J_{C,P}=6.0 Hz), 37.9 (d, J_{C,P}=7.6 Hz), 21.7; ³¹P NMR (162 MHz, D₂O) δ −6.22 (d, J=21.6 Hz), −9.54 (d, J=21.5 Hz); HRMS m/z calculated for C₅H₁₂O₇P₂ [M−H⁺]⁻ 244.9985, found: 244.9985.

But-3-en-1-yl diphosphate (18): ¹H NMR (400 MHz, D₂O) δ 5.93-5.78 (m, 1H), 5.18-5.10 (m, 1H), 5.06 (ddd, J=10.4, 2.2, 1.1 Hz, 1H), 3.95 (dt, J_{H,P}=1.0 Hz, J_{H,H}=6.7, 2H), 2.37 (dt, J=6.7, 6.4 Hz, 2H); ¹³C NMR (101 MHz, D₂O) δ 135.4, 116.9, 64.7, 34.5 (d, J_{C,P}=7.2 Hz); ³¹P NMR (162 MHz, D₂O) δ −7.03 (d, J=21.7 Hz), −9.52 (d, J=21.7 Hz); HRMS m/z calculated for C₄H₁₀O₇P₂ [M−H⁺]⁻ 230.9829, found: 230.9831.

Pent-4-en-2-yl diphosphate (19): ¹H NMR (400 MHz, D₂O) δ 5.88 (ddt, J=17.3, 10.3, 7.1 Hz, 1H), 5.22-5.04 (m, 2H), 4.44-4.33 (m, 1H), 2.36 (dq, J=25.1, 7.2 Hz, 2H), 1.25 (d, J=6.3 Hz, 3H); ¹³C NMR (101 MHz, D₂O) δ 134.9, 117.6, 73.2 (d, J_{C,P}=5.7 Hz), 41.4, 20.45; ³¹P NMR (162 MHz, D₂O) δ −8.96 (d, J=20.5 Hz), −11.51 (d, J=20.6 Hz); HRMS m/z calculated for C₅H₁₂O₇P₂ [M−H⁺]⁻ 244.9985, found: 244.9987.

3-Bromobut-3-en-1-yl diphosphate (20): ¹H NMR (400 MHz, D₂O) δ 5.80 (s, 1H), 5.56 (s, 1H), 4.10 (dd, J_{H,P}=6.8 Hz, J_{H,H}=6.2 Hz, 2H), 2.78 (t, J=6.2 Hz, 2H); ¹³C NMR (101 MHz, D₂O) δ 129.9, 119.4, 63.6 (d, J_{C,P}=5.6 Hz), 41.70; ³¹P NMR (162 MHz, D₂O) δ −8.05 (d, J=21.4 Hz), −10.72 (d, J=21.1 Hz); HRMS m/z calculated for C₄H9BrO₇P2 [M−H⁺]⁻ 308.8935, found: 308.8940.

Pent-4-yn-1-yl diphosphate (21): ¹H NMR (400 MHz, D₂O) δ 5.85-5.98 (s, 1H), 5.24-4.98 (m, 2H), 3.95-3.90 (m, 2H), 2.14 (q, J=7.9 Hz, 2H), 1.72 (t, J=4.0 Hz, 2H); ¹³C NMR (101 MHz, D₂O) δ 85.1, 69.5, 65.1, 28.8, 14.3; ³¹P NMR (162 MHz, D₂O) δ −7.03 (d, J=20.1 Hz), −9.72 (d, J=21.9 Hz); HRMS m/z calculated for C₅H₁₀O₇P₂ [M−H⁺]⁻ 242.9828, found 242.9830.

2-Methylallyl diphosphate (22): ¹H NMR (400 MHz, D₂O) δ 5.06 (s, 1H), 4.93 (s, 1H), 4.35 (d, J=7.0 Hz, 2H), 1.75 (s, 3H); ¹³C NMR (101 MHz, D₂O) δ 142.3, 111.4, 69.2, 18.4; ³¹P NMR (162 MHz, D₂O) δ −7.13 (d, J=20.8 Hz), −9.61 (d, J=20.9 Hz); HRMS m/z calculated for C₄H₁₀O₇P₂ [M−H⁺]⁻ 230.9829, found: 230.9824.

Pent-4-en-1-yl diphosphate (23): ¹H NMR (400 MHz, D₂O) δ 5.98-5.83 (m, 1H), 5.07 (d, J=17.4 Hz, 1H), 4.98 (d, J=10.5 Hz, 1H), 3.91 (dt, J_{H,P}=6.6 Hz, J_{H,H}=3.3 Hz, 2H), 2.12 (dt, J=7.5, 7.4 Hz, 2H), 1.78-1.62 (m, 2H); ¹³C NMR (101 MHz, D₂O) δ 139.1, 114.8, 65.8, 29.4, 29.2 (d, J_{C,P}=7.3 Hz); ³¹P NMR (162 MHz, D₂O) δ −6.71 (d, J=21.2 Hz), −9.38 (d, J=21.5 Hz); HRMS m/z calculated for C₅H1207P2 [M−H⁺]⁻ 244.9985, found: 244.9988.

(Z)-Hex-2-en-1-yl diphosphate (24): ¹H NMR (400 MHz, D₂O) δ 5.74-5.55 (m, 2H), 4.50 (dd, J_{H,P}=6.8 Hz, J_{H,H}=2.1 Hz, 2H), 2.12-2.02 (m, 2H), 1.36 (dq, J=13.3, 7.4, Hz, 2H), 0.86 (t, J=7.4 Hz, 3H); ¹³C NMR (101 MHz, D₂O) δ 135.2, 125.3 (d, J_{C,P}=8.0 Hz), 61.9, 29.0, 22.2, 13.1; ³¹P NMR (162 MHz, D₂O) δ −7.08 (d, J=21.4 Hz), −9.42 (d, J=21.3 Hz); HRMS m/z calculated for C₆H₁₄O₇P₂ [M−H⁺]⁻ 259.0142, found: 259.0145.

(E)-Hex-2-en-1-yl diphosphate (25): ¹H NMR (400 MHz, D₂O) δ 5.85 (dt, J=14.0, 6.8 Hz, 1H), 5.64 (dt, J=14.0, 6.4 Hz, 1H), 4.36 (dd, J_{H,P}=6.8 Hz, J_{H,H}=6.8 Hz, 2H), 2.02 (dt, J=6.4, 7.4 Hz, 2H), 1.36 (dtd, J=7.4, 7.3 Hz, 2H), 0.85 (td, J=7.3 Hz, 3H); ¹³C NMR (101 MHz, D₂O) δ 136.2, 125.6, 66.9 (d, J_{C,P}=5.4 Hz), 33.8, 21.7, 13.1; ³¹P NMR (162 MHz, D₂O) δ −6.92 (d, J=21.4 Hz), −9.55 (d, J=21.4 Hz); HRMS m/z calculated for C₆H₁₄O₇P₂ [M−H⁺]⁻ 259.0142, found: 259.0145.

But-3-yn-1-yl diphosphate (26): ¹H NMR (400 MHz, D₂O) δ 3.85 (dt, J_{H,P}=6.8 Hz, J_{H,H}=6.8 Hz, 2H), 2.44 (m, 2H), 2.13 (t, J=2.7 Hz, 1H); ¹³C NMR (101 MHz, D₂O) δ 82.1, 70.6, 64.1 (d, J_{C,P}=96.6, 5.5 Hz), 20.2; ³¹P NMR (162 MHz, D₂O) δ −7.82 (d, J=20.4 Hz), −9.94 (d, J=20.9 Hz); HRMS m/z calculated for C₄H₈O₇P₂ [M−H⁺]⁻ 228.9672, found: 228.9675.

Cinnamyl diphosphate (27): ¹H NMR (400 MHz, D₂O) δ 7.54-7.49 (m, 1H), 7.39 (td, J=7.5, 1.8 Hz, 1H), 7.34-7.30 (m, 1H), 6.60 (d, J=15.2, Hz, 1H), 6.30 (dt, J=15.2, 6.2, Hz, 1H), 4.50 (dd, J_{H,P}=6.2 Hz, J_{H,H}=6.2 Hz, 2H); ¹³C NMR (101 MHz, D₂O) δ 136.4, 132.2, 128.9, 126.6, 125.6 (d, J_{C,P}=8.0 Hz), 66.5 (d, J_{C,P}=5.1 Hz); ³¹P NMR (162 MHz, D₂O) δ −6.98 (d, J=21.0 Hz), −9.50 (d, J=20.8 Hz); HRMS m/z calculated for C₉H₁₂O₇P₂ [M−H⁺]⁻ 292.9985, found: 292.9991.

Hept-3-yn-1-yl diphosphate (28): ¹H NMR (400 MHz, D₂O) δ 4.00-3.85 (m, 2H), 2.47 (t, J=6.5 Hz, 2H), 2.08 (d, J=7.4 Hz, 2H), 1.42 (tq, J=7.4, 7.3 Hz, 2H), 0.88 (t, J=7.3 Hz, 3H); ¹³C NMR (101 MHz, D₂O) δ 83.3, 77.6, 64.4 (d, J_{C,P}=5.0 Hz), 23.3, 21.7, 20.4 (d, J_{C,P}=Hz), 20, 12.9; ³¹P NMR (162 MHz, D₂O) δ −6.91 (d, J=21.1 Hz), −9.79 (d, J=21.2 Hz); HRMS m/z calculated for C₇H₁₄O₇P₂ [M−H⁺]⁻ 271.0142, found: 271.0146.

2-(Thiophen-3-yl)ethyl diphosphate (29): ¹H NMR (400 MHz, D₂O) δ 7.20 (dd, J=4.9, 3.0 Hz, 1H), 7.04 (dd, J=3.0, 1.3 Hz, 1H), 6.92 (dd, J=4.9, 1.3 Hz, 1H), 3.93 (dt, J_{H,P}=7.0 Hz, J_{H,H}=6.6 Hz, 2H), 2.79 (t, J=6.6 Hz, 2H); ¹³C NMR (101 MHz, D₂O) δ 138.8, 128.6, 125.9, 121.8, 65.7 (d, J_{C,P}=5.8 Hz), 30.6 (d, J_{C,P}=7.6 Hz); ³¹P NMR (162 MHz, D₂O) δ −7.19 (d, J=20.9 Hz), −10.53 (d, J=21.0 Hz); HRMS m/z calculated for C₆H₁₀O₇P₂S [M−H⁺]⁻ 286.9550, found: 286.9554.

Furan-3-ylmethyl diphosphate (30): ¹H NMR (400 MHz, D₂O) δ 7.50 (d, J=3.2 Hz, 1H), 6.49 (dd, J=3.7, 3.2 Hz, 1H), 6.41 (d, J=3.7 Hz, 1H), 4.88 (d, J_{H,P}=6.8 Hz, 1H); ¹³C NMR (101 MHz, D₂O) δ 150.7, 143.7, 110.8, 110.1, 59.7 (d, J_{C,P}=5.3 Hz); ³¹P NMR (162 MHz, D₂O) δ −6.62 (d, J=21.8 Hz), −9.91 (d, J=21.3 Hz); HRMS m/z calculated for C₅H₈O₈P₂ [M−H⁺]⁻ 256.9622, found: 256.9626.

But-2-yn-1-yl diphosphate (31): ¹H NMR (400 MHz, D₂O) δ 3.17 (dd, J_{H,P}=7.4 Hz, J_{H,H}=7.3 Hz, 2H), 1.25 (t, J=7.3 Hz, 3H); ¹³C NMR (101 MHz, D₂O) δ 79.8 (d, J_{C,P}=9.5 Hz), 75.8, 54.2 (d, J_{C,P}=4.6 Hz), 1.7; ³¹P NMR (162 MHz, D₂O) δ −6.13 (d, J=21.5 Hz), −9.78 (d, J=21.6 Hz); HRMS m/z calculated for C₄H₈O₇P₂ [M−H⁺]⁻ 228.9672; found: 228.9673.

Pyridin-3-ylmethyl diphosphate (32): ¹H NMR (400 MHz, D₂O) δ 8.54 (s, 1H), 8.43 (d, J=5.0 Hz, 1H), 7.97 (d, J=8.1 Hz, 1H), 7.35-7.26 (m, 1H), 5.02 (d, J_{H,P}=7.4 Hz, 2H); ¹³C NMR (101 MHz, D₂O) δ 147.0, 146.5, 138.1, 134.6, 124.6, 64.9 (d, J_{C,P}=5.0 Hz); ³¹P NMR (162 MHz, D₂O) δ −6.79 (d, J=21.5 Hz), −9.75 (d, J=21.4 Hz); HRMS m/z calculated for C₆H9NO₇P₂ [M−H⁺]⁻ 267.9781, found: 267.9785.

Hex-5-yn-1-yl diphosphate (33): ¹H NMR (400 MHz, D₂O) δ 3.86 (dt, J_{H,P}=6.4 Hz, J_{H,H}=6.4 Hz, 2H), 2.32 (d, J=2.7 Hz, 1H), 2.28-2.17 (m, 2H), 1.76-1.65 (m, 2H), 1.62-1.52 (m, 2H); ¹³C NMR (101 MHz, D₂O) δ 85.8, 69.2, 65.2 (d, J_{C,P}=5.5 Hz), 28.8 (d, J_{C,P}=7.0 Hz), 24.0, 17.1; ³¹P NMR (162 MHz, D₂O) δ −6.58 (d, J=22.1 Hz), −10.66 (d, J=22.1 Hz); HRMS m/z calculated for C₆H₁₂O₇P₂ [M−H⁺]⁻ 256.9985, found: 256.9989.

2-(Allyloxy)ethyl diphosphate (34): ¹H NMR (400 MHz, D₂O) δ 5.93 (ddt, J=13.6, 7.2, 3.9 Hz, 1H), 5.32 (dt, J=13.6, 2.1 Hz, 1H), 5.28-5.20 (m, 1H), 4.08-3.95 (m, J=10.4, 7.9, 2.6 Hz, 4H), 3.75-3.68 (m, 2H); ¹³C NMR (101 MHz, D₂O) δ 133.6, 118.7, 90.3, 73.1, 69.4 (d, J_{C,P}=7.3 Hz); ³¹P NMR (162 MHz, D₂O) δ −9.46 (d, J=25.1 Hz), −9.74 (d, J=25.1 Hz); HRMS m/z calculated for C₅H₁₂O₈P₂ [M−H⁺]⁻ 260.9935, found: 260.9937.

2-(1H-Indol-3-yl)ethyl diphosphate (35): ¹H NMR (400 MHz, D₂O) δ 7.65 (d, J=8.1 Hz, 1H), 7.41 (d, J=8.1 Hz, 1H), 7.23 (s, 1H), 7.15 (t, J=7.6 Hz, 1H), 7.07 (t, J=7.6 Hz, 1H), 4.12 (dd, J_{H,P}=7.3 Hz, J_{H,H}=7.0 Hz, 2H), 3.05 (t, J=7.0 Hz, 2H); ¹³C NMR (101 MHz, D₂O) δ 136.1, 127.0, 123.8, 121.9, 119.2, 118.8, 111.9, 111.22, 66.0 (d, J_{C,P}=2.6 Hz), 26.0; ³¹P NMR (162 MHz, D₂O) δ −5.63 (d, J=21.6 Hz), −9.34 (d, J=21.6 Hz); HRMS m/z calculated for C₁₀H₁₃NO₇P₂ [M−H⁺]⁻ 320.0094, found: 320.0098.

Prop-2-yn-1-yl diphosphate (36): ¹H NMR (400 MHz, D₂O) δ 4.40 (d, J_{H,P}=7.8 Hz, 2H), 2.57 (s, 1H); ¹³C NMR (101 MHz, D₂O) δ 77.9, 75.9, 52.6 (d, J_{C,P}=7.8 Hz); ³¹P NMR (162 MHz, D₂O) δ −7.69 (d, J=20.8 Hz), −10.11 (d, J=20.8 Hz); HRMS m/z calculated for C₃H₆O₇P₂ [M−H⁺]⁻ 214.9516, found: 214.9519.

3,7-dimethyloct-6-en-1-yl diphosphate (37): ¹H NMR (400 MHz, D₂O) δ 5.21 (t, J=7.4 Hz, 1H), 3.99-3.88 (m, 2H), 2.07-1.91 (m, 2H), 1.66 (s, 3H), 1.59 (s, 3H), 1.49-1.37 (m, 2H), 1.38-1.26 (m, 2H), 1.21-1.19 (m, J=6.3, 1.5 Hz, 1H), 0.87 (d, J=6.3 Hz, 3H); ¹³C NMR (101 MHz, D₂O) δ 133.0, 125.2, 64.8 (d, J_{C,P}=5.8 Hz), 36.7 (d, J_{C,P}=7.3 Hz), 36.3, 24.7, 24.7, 23.1, 18.5, 16.8; ³¹P NMR (162 MHz, D₂O) δ −7.08 (d, J=17.7 Hz), −9.39 (d, J=21.4 Hz); HRMS m/z calculated for C₁₀H₂₂O₇P₂ [M−H⁺]⁻ 315.0768, found: 315.0770.

(E)-3,7-Dimethylocta-2,6-dien-1-yl diphosphate (geranyl diphosphate, GPP): ¹H NMR (400 MHz, D₂O) δ 5.17 (t, J=6.0 Hz, 1H), 5.00-4.82 (m, 1H), 4.20 (dt, J_{H,P}=7.2 Hz, J_{H,H}=6.0 Hz, 2H), 1.92-1.72 (m, 4H), 1.44 (s, 3H), 1.41 (s, 3H), 1.34 (s, 3H); ¹³C NMR (101 MHz, D₂O) δ 142.8, 133.2, 124.1, 119.7 (d, J_{C,P}=8.4 Hz), 62.8 (d, J_{C,P}=5.3 Hz), 39.0, 25.8, 25.0, 17.1, 15.7; ³¹P NMR (162 MHz, D₂O) δ −8.18 (d, J=20.2 Hz), −9.59 (d, J=20.7 Hz); HRMS m/z calculated for C₁₀H₂₀O₇P₂ [M−H⁺]⁻ 313.0611, found: 313.0616.

7-methyloct-6-en-3-yn-1-yl diphosphate (GPP-26a): ¹H NMR (400 MHz, D₂O) δ 5.05 (tdd, J=5.5, 2.0, 1.5 Hz, 1H), 3.79 (dt, J_{H,P}=6.8 Hz, J_{H,H}=6.8 Hz J=6.9, 2H), 2.77-2.66 (m, 2H), 2.35 (td, J=6.9, 3.6 Hz, 2H), 1.53 (s, 3H), 1.46 (s, 3H); ¹³C NMR (101 MHz, D₂O) δ 135.7, 118.7, 81.5, 77.2, 64.3 (d, J_{C,P}=5.8 Hz), 24.8, 20.5, 17.1, 16.9; ³¹P NMR (162 MHz, D₂O) δ −6.95 (d, J=25.6 Hz), −9.85 (d, J=21.5 Hz); HRMS m/z calculated for C₉H₁₆O₇P₂ [M−H⁺]⁻ 297.0298, found: 297.0299.

7-methylocta-2,3,6-trien-1-yl diphosphate (GPP-26b): ¹H NMR (700 MHz, D₂O) δ 5.32 (m, 2H), 5.26-5.21 (m, 1H), 4.31 (dd, J_{H,P}=8.2 Hz, J_{H,H}=6.8 Hz, 2H), 2.70 (d, J=7.7 Hz, 2H), 1.70-1.66 (m, 3H), 1.60 (s, 2H); ¹³C NMR (176 MHz, D₂O) δ 203.1, 133.6, 119.9, 90.6, 87.5, 62.7 (d, J_{C,P}=6.4 Hz), 25.1, 23.3, 15.5; ³¹P NMR (162 MHz, D₂O) δ −6.79 (d, J=20.4 Hz), −9.83 (d, J=21.8 Hz); [α]D₂₅=2.9°; HRMS m/z calculated for C₉H₁₆O₇P₂ [M−H⁺]⁻ 297.0298, found: 297.0291.

TABLE 6
Masses of detected GPP analogues from extender unit analogue reactions
with IspA and DMAPP.
GPP Analogues

| | Chemical | Calculated Mass | Found | Mass error |
|---|---|---|---|---|
| Extender | Formula | [M − H]⁻ | [M − H]⁻ | (ppm) |
| 18 | C₉H₁₈O₇P₂ | 299.0455 | 299.0459 | 1.5 |
| 20 | C₉H₁₇BrO₇P₂ | 376.9561 | 376.9568 | 2.2 |
| 21 | C₁₀H₁₈O₇P₂ | 311.0455 | 311.0463 | 2.6 |
| 22 | C₉H₁₈O₇P₂ | 299.0455 | 299.0460 | 1.6 |
| 23 | C₁₀H₂₀O₇P₂ | 313.0611 | 313.0620 | 2.7 |
| 26 | C₉H₁₆O₇P₂ | 297.0298 | 297.0307 | 2.7 |
| 28 | C₁₂H₂₂O₇P₂ | 339.0768 | 339.0779 | 3.4 |
| 29 | C₁₁H₁₈O₇P₂S | 355.0176 | 355.0185 | 2.6 |
| 31 | C₉H₁₆O₇P₂ | 297.0298 | 297.0305 | 2.3 |
| 33 | C₁₁H₂₀O₇P₂ | 325.0611 | 325.0621 | 3.0 |

TABLE 7
Masses of detected FPP analogues from extender unit analogue reactions
with IspA and GPP.
FPP Analogues

| | Chemical | Calculated Mass | Found | Mass error |
|---|---|---|---|---|
| Extender | Formula | [M − H]⁻ | [M − H]⁻ | (ppm) |
| 18 | C₁₄H₂₆O₇P₂ | 367.1081 | 367.1083 | 0.5 |
| 20 | C₁₄H₂₅BrO₇P₂ | 445.0187 | 445.0185 | 0.3 |
| 21 | C₁₅H₂₆O₇P₂ | 379.1081 | 379.1083 | 0.5 |
| 22 | C₁₄H₂₆O₇P₂ | 367.1081 | 367.1084 | 0.8 |
| 26 | C₁₄H₂₂O₇P₂ | 365.0924 | 365.0925 | 0.2 |
| 29 | C₁₆H₂₆O₇P₂S | 423.0802 | 423.0801 | 0.2 |
| 33 | C₁₆H₂₈O₇P₂ | 393.1237 | 393.1231 | 1.6 |

### Example 5. Further Efforts—Applications of Unnatural Terpene Precursors

Unnatural terpenes have been used in a variety of reactions and applications. Initially, terpene analogues were applied as substrate mimics for the purpose of developing inhibitors. For instance, bisphosphonate drugs are substrate mimics that are used to target enzymes utilizing pyrophosphorylated substrates.

Terpene analogues have also been used in a variety of chemical biology studies. Researchers use farnesyl pyrophosphate analogues that contain Click handles for in vivo studies of protein farnesylation which is used to study ubiquitination pathways in cancer. More recently, terpene analogues have been used to study mechanisms utilized by terpene cyclases. Such fluorinated analogues of substrates for terpene cyclases halt reaction cascade processes by restricting hydride shifts and deprotonation events that may occur in the process of product maturation.

Generation of novel substrates for studying terpene cyclases and protein prenyltransferases has previously been accomplished through traditional organic synthesis in lieu of suitable biosynthetic approaches. While organic synthesis has generated a modest albeit limited pool of rationally designed potential substrates, biosynthesis of non-natural and/or non-native precursors has been the subject of only limited inquiry.

The platform presented herein provides a means by which a plethora of opportunities to diversify terpenes using a synthetic biology approach can be accomplished

Unnatural Diphosphate Cyclization

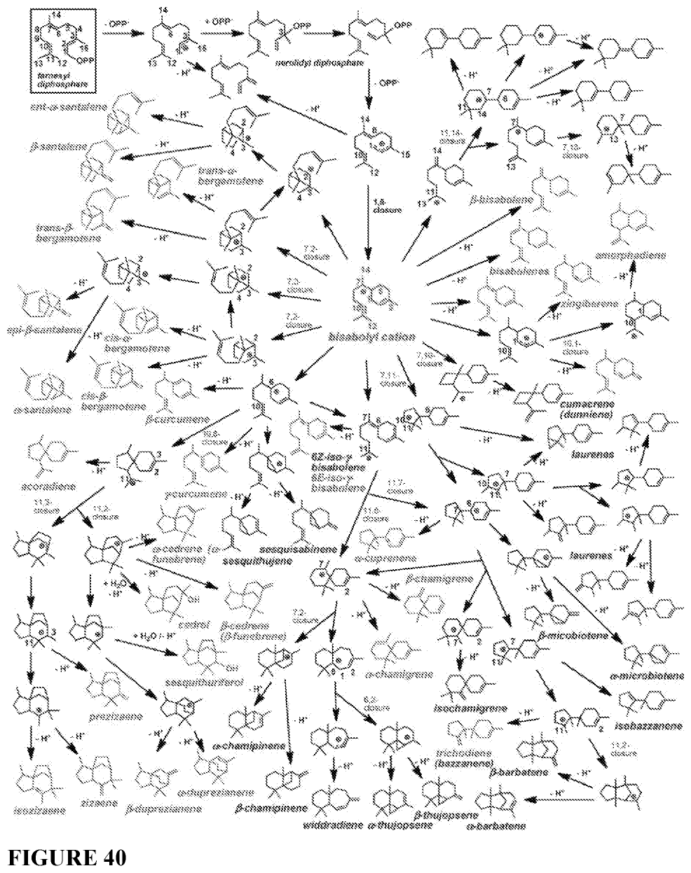

Preliminary investigation of terpene cyclase activity. Terpene cyclases afford incredible molecular diversity from relatively simply starting materials. Using carbocationic chemistry, these enzymes elegantly direct complex cascade reactions by navigating high energy intermediates to complex ring structures (FIG. 40). Prior to investigating the ability of these enzymes to use previously generated FPP analogues, confirmation of activity in vivo is needed.

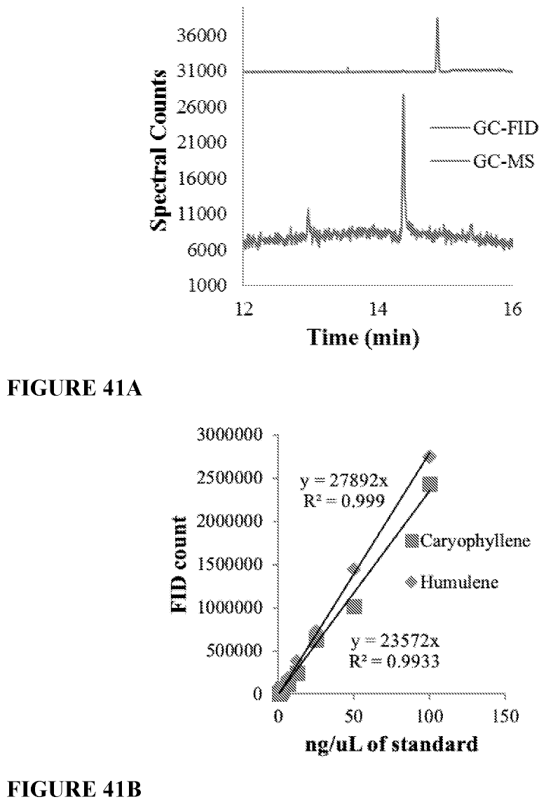

Before embarking on measuring activity in vitro, an analytical method had to be developed in order to minimize detection limits to assure that if unnatural terpenes were generated, even the smallest amount could be detected. GC-FID was qualitatively compared to GC-MS for detection limits and signal to noise (FIGS. 41A-41B). Using standards, GC-FID, a minimum detection limit of 390 ng/uL was detected using trans-caryophyllene and γ-humulene. The precursor diphosphates themselves cannot be detected by GC as the boiling points and stability are not suitable for GC. Together, the best manner for detecting unnatural terpene cyclization would be to first screen for activity using a higher sensitivity method using FID followed by EI-MS for potential structure identification.

Following this result, it was then tested to see if terpenes could be detected from in vitro reactions. Aristolochene synthase was expressed from ATAS. Aristolochene synthase was then tested with synthesized FPP to observe the natural reaction in vitro and products were confirmed by EI-MS using the NIST database.

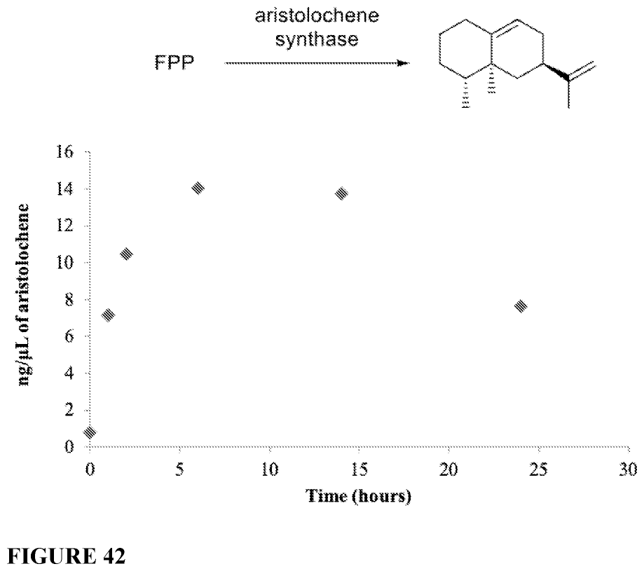

Following the confirmation of activity in vitro, production overtime was then measured using FID (FIG. 42).

These results cumulatively suggest a work flow for screening terpene cyclases. 1) Semi-purify terpene cyclases to increase the concentration of catalyst. 2) Screen for product cyclization using a high sensitivity method (FID). 3) Confirm product identity by GC-MS. 4) Elucidate structure by isolation and NMR.

Unnatural diphosphates as substrates for terpene cyclases. In vitro studies in prior examples have shown the production of unnatural farnesyl and geranyl diphosphates. To generate unnatural terpenes, the analogues afforded through incorporation of unnatural substrates into GPP and FPP analogues using IspA must be coupled with terpene cyclases.

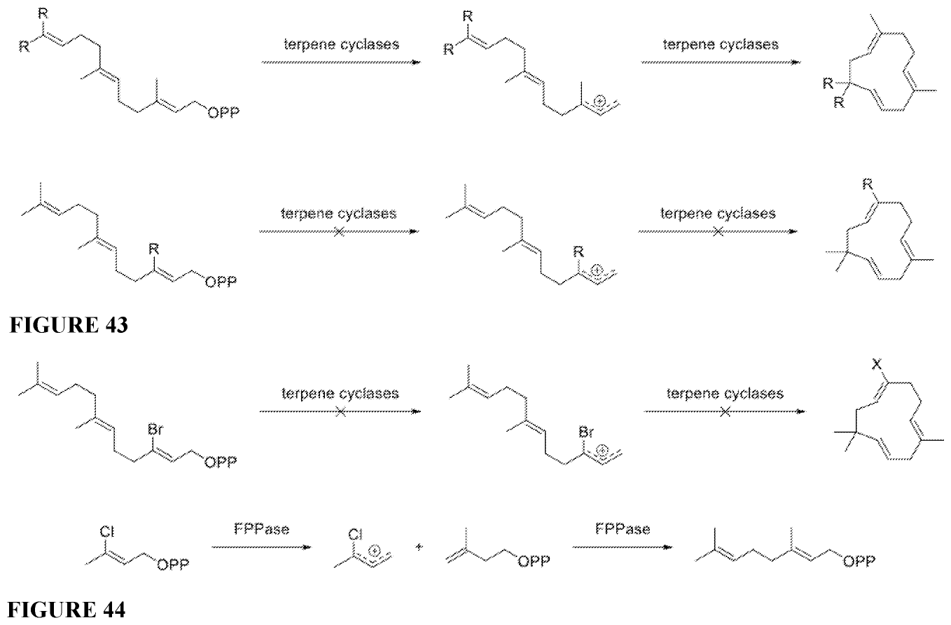

These analogues may be incorporated into cyclic structures by terpene cyclases, but also may require enzyme engineering for few possible reasons. 1) The substrates did not contain the seemingly requisite allylic diphosphate moiety. This would require the engineering of a terpene cyclase capable of directing an intramolecular S_{N}2 reaction for the release of diphosphate. 2) Terpene cyclases have a strict substrate specificity at the head of the diphosphates (FIG. 43). If not limited by energetics, rational engineering may sterically permit analogues in place of the natural substrates.

Attempts to cyclize substrates diversified at the head portion of the molecule have been limited. In one example, attempts were made to cyclize a halogenated derivative of FPP, but no ionization was detected. While it was found that the halogenated analogue directly inhibited the terpene cyclase, it was hypothesized that ionization of the halogenated analogue wasn't energetically feasible as the resulting allylic carbocation was too high in energy. In direct opposition to this assertion, halogenated DMAPP analogues have been used for prenyltransferase reactions. As these reactions would proceed through similar intermediates, engineering of terpene cyclases to accept halogenated derivatives should be feasible (FIG. 44).

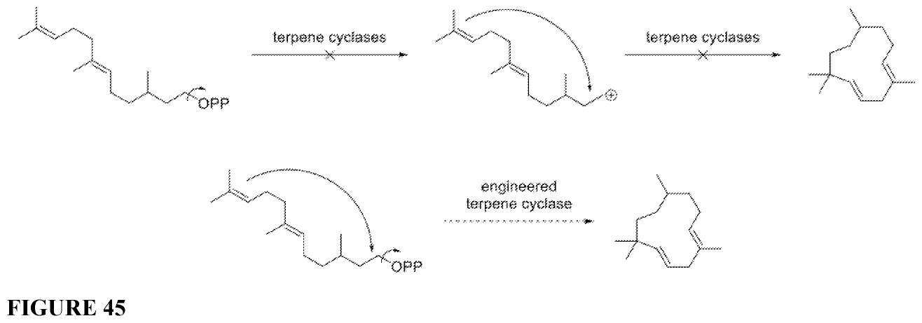

Another effort that would address these issues of substrate specificity would be screening for a terpene cyclase capable of cyclization of non-allylic diphosphates. An enzyme capable of cyclizing such a substrate would proceed through a concerted reaction mechanism which would surpass the requirement of an allylic carbocation (FIG. 45).

Transfer of Non-Natural Prenyl Donors to Aromatic Acceptors

In vitro studies of aromatic prenyltransferases. Beyond use of hemiterpenes for elongation and subsequent cyclization by terpene cyclases, hemiterpenes can be appended to a variety of other natural products. Meroterpenoids are natural products such as polyketides or non-ribosomal peptides that have been prenylated. The prenyl groups often are critical for bioactivity as the prenyl side chains alter C log P values to increase bioavailability.

ABBA aromatic prenyltransferases are a class of soluble magnesium-independent enzymes that append prenyl groups to various aromatic prenyl acceptors. These enzymes have been noted for their broad promiscuity in terms of prenyl acceptors, but less so in terms of prenyl donors.

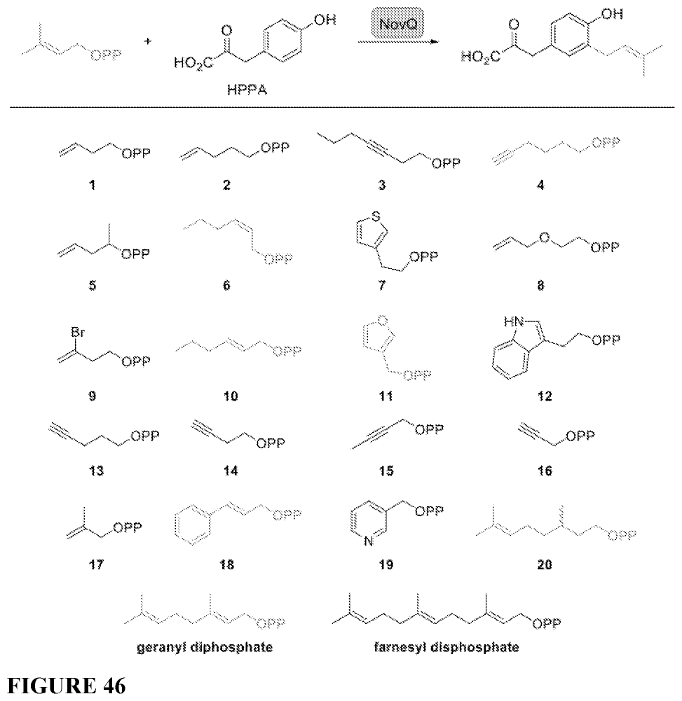

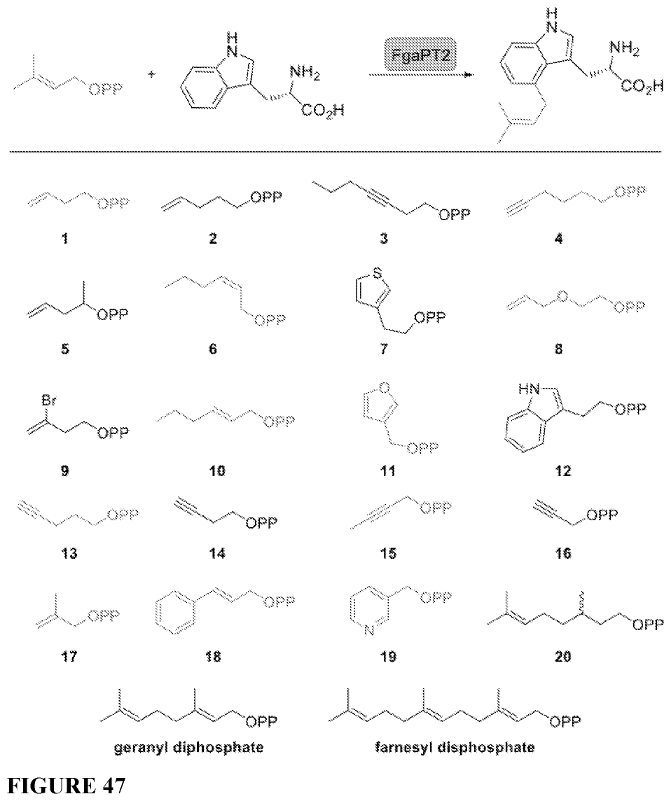

From the enzymes studied to date, prenyltransferases have shown remarkable promiscuity in terms of prenyl identity suggesting some catalytic flexibility never before noted (FIG. 46 and FIG. 47).

As many of the substrate utilized do not contain an allylic diphosphate, this work cumulatively suggests that ABBA prenyltransferases can utilize a concerted mechanism for prenylation. This mechanistic tolerance of analogues suggests that these catalysts can be used for general C—C bond formation between various alkyl diphosphates and aromatic structures.

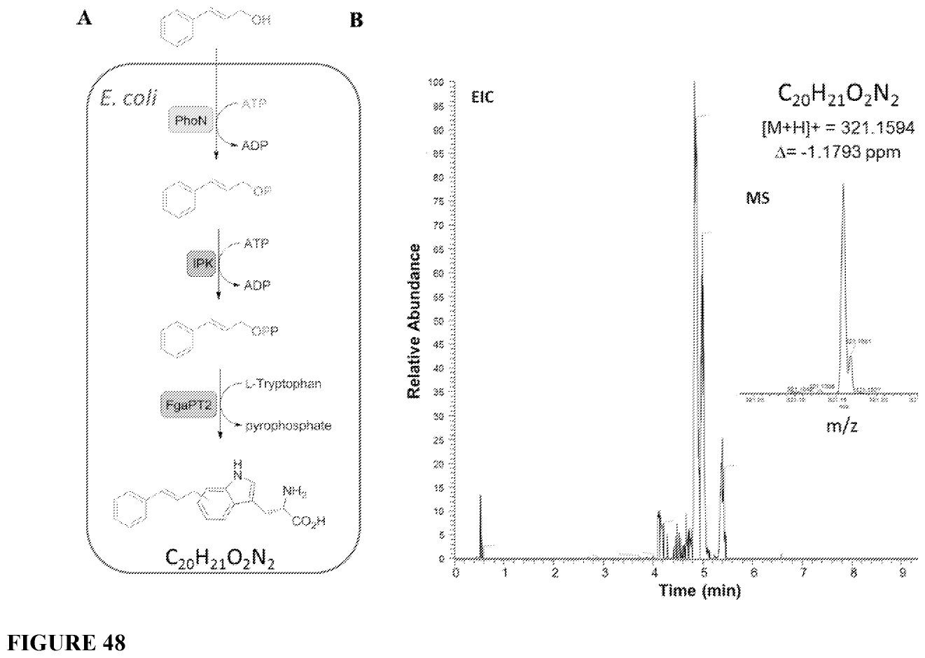

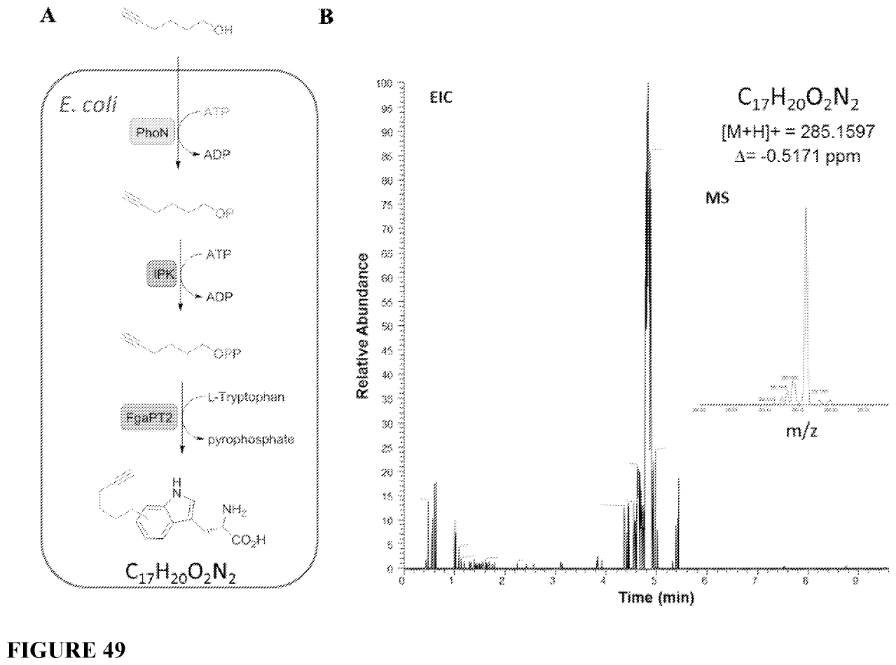

In vivo studies of aromatic prenyltransferases. As several aromatic prenyltransferases were shown to be promiscuous in vitro, we next evaluated if these activities could be coupled with the putative hemiterpene analogue production platform in vivo. The system consisted of BL21 DE3 harboring pETDuet-PhoN+IPK and pET28a-FgaPT2. Analogues found to be substrates in vitro for all three enzymes were the candidates for analoging in vivo. Briefly, cells containing the vectors and single enzyme omission controls were grown to a density of OD₆₀₀=0.6 before protein expression was induced by the addition of IPTG. At this same point, substrate was provided to the cultures and the fermentation was allowed to proceed for 48 hours. Aliquots were taken and the cell lysate was examined by high resolution LC-MS (FIG. 48 and FIG. 49)

Once assembled in vivo, the parts functioned as found in vitro. The hemiterpene production pathway was successfully coupled with FgaPT2 to generate two tryptophan derivatives in vivo. Controls omitting any of the enzymes or substrate did not provide any product consistent with the tryptophan derivatives generated in vitro. When the hemiterpene production pathway was used with dimethylallyl alcohol, as was done with the carotenoid assay, prenylated tryptophan was observed at twice the concentration of that observed without the artificial pathway. While these vectors were not optimized for in vivo production, analogues were detected nonetheless. Future studies will use compatible vectors to increase analogue production for scale-up, isolation, and structural characterization.

ABBA Prenyltransferases with IspA Generated Terpene Analogues

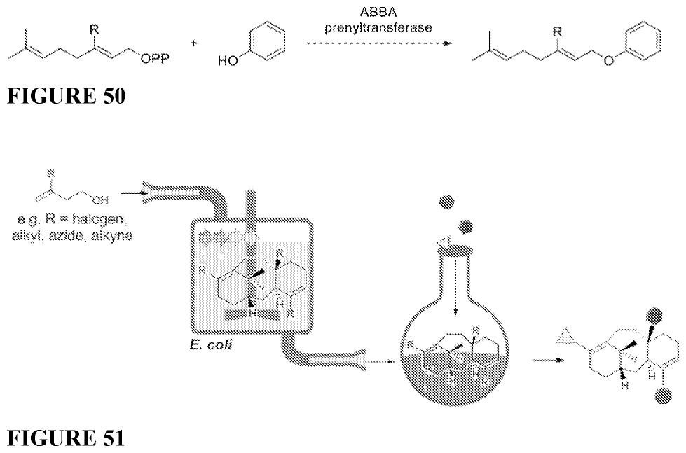

Isolation of the prenyltransferase generated GPP and FPP analogues is very difficult due to the ease of product decomposition, challenging separation, and the preparation of starting material. Stemming from the promiscuity observed with the ABBA prenyltransferases, it is envisioned that the mechanistic plasticity of these prenyltransferases can be used for the prenylation of aromatic systems using these unnatural prenyl donors generated by IspA (FIG. 50).

Using a sufficiently promiscuous prenyltransferase for the addition of unnatural prenyl groups to aromatic systems should ease difficulties with isolation as the prenylated aromatic rings are much more stable and easier to separate by chromatography. Ideally such a system could be coupled in vivo for scale up.

Conclusions

The work outlined in this example sets the foundation for the biosynthesis of hemiterpene analogues from chemical precursors. These precursors show broad application for potential use in terpene and prenylated natural product derivatives. This can provide non-natural chemical handles for synthetic diversification not available to the native products themselves (FIG. 51).

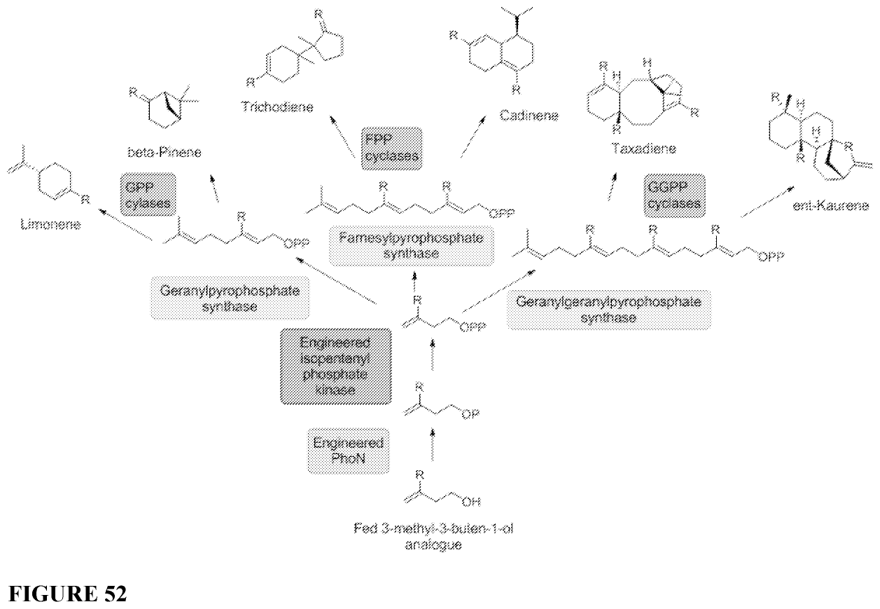

By validating this platform part by part in vitro, the promiscuity and limits of each catalyst is more fully realized. Enzymes such as the kinases as PhoN from S. flexneri and IPK from T. acidophilum carry out simple phosphorylations and appear to have a broad range in substrate specificity based on the simplicity of the chemical transformation and mechanism. While IspA catalyzes several distinct steps to elongate prenyl groups, promiscuity towards non-natural nucleophiles is observed. While no success was had in generating cyclic terpene analogues, these enzymes can be engineered by methodically screening for use of target substrates. Perhaps another application of terpene analogues is their appendage to aromatic rings as is observed with the aromatic prenyltransferases NovQ and FgaPT2 This work lays the foundation and validates use of this platform as a path for precursor directed terpene diversification that has not yet been investigated and attempted for this class of natural products (FIG. 52).

Methods

General methods and material. All plasmids were verified by DNA sequencing. Purifications of all DNA were performed with kits from BioBasic. Synthetic oligonucleotides were purchased from IDT (Coralville, Iowa, USA). Restriction enzymes were purchased from New England Biolabs (Ipswich, Mass., USA). Polymerase chain reactions were conducted using Phire Hot Start II DNA Polymerase from ThermoFisher Scientific (Waltham, Mass., USA). Standards of trans-caryophyllene and α-humulene as well as farnesyl pyrophosphate lithium salt were purchased from Sigma Aldrich (St. Louis, Mo., USA).

Expression and purification of IspA. ATAS plasmid was transformed into E. coli BL21 (DE3) for protein expression. A single colony was used to inoculate a 3 mL culture in LB media supplemented with chloramphenicol (35 μg/mL). A 1 L culture containing chloramphenicol (35 μg/mL) in LB media was then inoculated with 1 mL of the overnight culture and grown to an OD600 of ˜0.6 at 37° C. with shaking at 300 rpm at which point protein expression was induced by the addition of 1 mM IPTG. The temperature of the incubator-shaker was reduced to 18° C. and the culture incubated for approximately 18 hours. The culture was pelleted at 4000 rpm for 10 mins, the supernatant was decanted, the cell pellet resuspended in 15 mL of lysis buffer (100 mM TRIS-HCl, 300 mM NaCl, 10% glycerol, pH 8.0) and lysed by sonication. The lysate was then pelleted at 4500 rpm for 10 mins, decanted, and the soluble protein was spun down at 15,000 rpm for 1 hour. The resulting soluble fraction was then purified by fast protein liquid chromatography (FPLC) using nickel-bead column chromatography for the extraction of His6-tagged proteins. The column was first equilibrated with wash buffer (50 mM TRIS-HCl, 500 mM NaCl, 20 mM imidazole, pH 8.0) prior to loading of the soluble fraction. The soluble fraction was then eluted with elution buffer (50 mM TRIS-HCl, 500 mM NaCl, 200 mM imidazole, pH 8.0) using a gradient of 0% elution buffer 0-7.5 min., 0-50% 7.5-18 min., 50-100% 18-22 min., 100% 22-27.5 min, and equilibrated for additional runs with 0% elution buffer 27.5-35 min. Fractions containing the desired protein were identified by SDS-PAGE and pooled. The pooled protein was then concentrated using a 10,000 molecular weight cut-off filter (Millipore Amicon-Ultra) and the buffer was exchanged with protein storage buffer (50 mM TRIS-HCl, 100 mM NaCl, and 20% glycerol at pH 8.0). Protein aliquots were flash frozen with a dry ice ethanol bath before storage at −80° C. Protein purity was confirmed by SDS-PAGE while concentration was determined by absorbance using a Pierce Bradford Protein Assay kit.

GC analysis of terpenes. Standards were diluted serially in ethyl acetate. GC-MS and GC-FID were performed on an Agilent Technologies 5975 GC/MS equipped with an HP-5MS capillary column (0.25 mm i.d., 30 m) (Agilent Technologies). The GC was operated using splitless injections at a volume of 1 μL with an injector temperature of 250° C. The initial oven temperature was set to 50° C. for 5 minutes before being ramped at 15° C./min to 230° C. and then held at 240° C. for 1 minute. Product peaks were integrated using Agilent ChemStation software.

Reactions of aristolochene synthase. Reactions were performed in 1.5 mL polypropylene tubes. Reactions were run in volumes of 200 μL overlaid with 200 μL of ethyl acetate. Reactions contained 2.5 mM farnesyl pyrophosphate, 2.5 mM MgCl2, 25 mM Tris-HCl at pH 7.5, and 7.8 μg of enzyme. Time points were taken by halting the reaction by vortexing the mixture and removing the ethyl acetate layer for subsequent analysis.

### Example 6. Further Applications of the Artificial Isoprenoid Pathway

The following data demonstrates the ability of the artificial isoprenoid pathway (PhoN-IPK) to support production of natural isoprenoids (via DMAPP/IPP) and non-natural isoprenoids (via various non-natural alkyl-pyrophosphates). The PhoN-IPK is coupled to various downstream enzymes (FgaPT2, IspA, CpaD, FtmPT1) to afford a range of compounds.

Production of prenylated amino acids in vitro via PhoN-IPK-FgaPT2 pathway. Reactions including purified PhoN, IPK, and FgaPT2 with ATP, DMAA, and Trp were run with initial conditions similar to individual enzyme reactions.

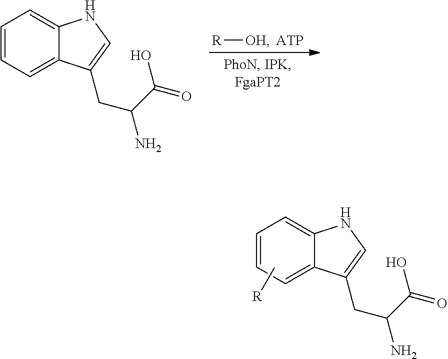

Buffer was optimized, followed by iterative optimization of substrate and enzyme concentrations. Reactions were followed by HPLC, and percent conversion was calculated in the same manner as FgaPT2 in vitro reactions. The results are shown in Table 8 below.

TABLE 8
In situ production of DMAPP from DMAA by PhoN and IPK followed by
use by FgaPT2. Concentration of reaction components were optimization
for higher percent conversion as detected by HPLC at 269 nm.

| | Best | | | |
|---|---|---|---|---|
| | Conditions | | Initial Conditions | |
| Component | Concentration | | Concentration | |
| Trp | 1 | mM | 1 | mM |
| ATP | 3.6 | mM | 2.5 | mM |
| R-OH | 70 | mM | 5 | mM |
| FgaPT2 | 350 | ng/uL | 200 | ng/uL |
| IPK | 50 | ng/uL | 20 | ng/uL |
| PhoN | 87 | ng/uL | 20 | ng/uL |

| | | |
|---|---|---|
| % Conversion | 80.1 | 1.6 |

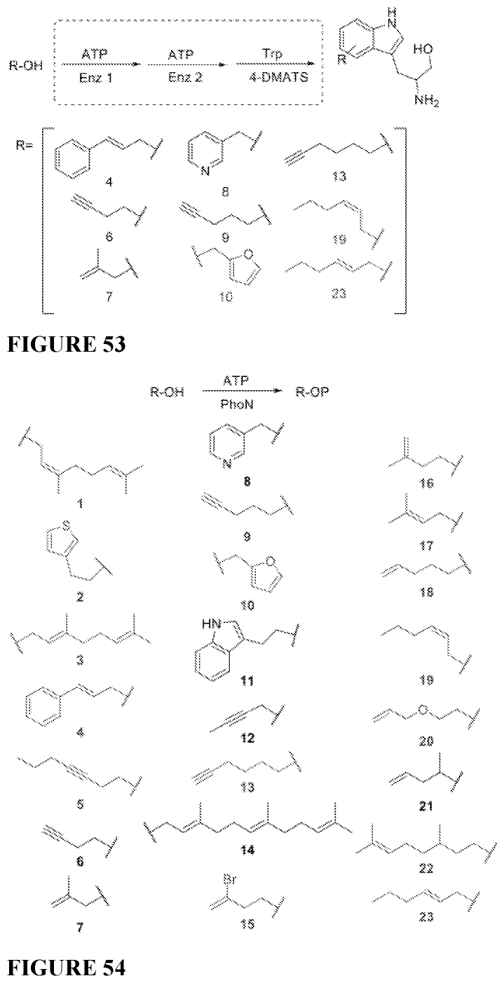

Production of prenylated amino acids in vivo via the PhoN-IPK, FgaPT2 pathway. Alcohols of interest (5 mM) were fed into cultures expressing FgaPT2, PhoN, and IPK in E. coli Rosetta PLysS. Media and cell lysate were analyzed by HPLC detecting at 269 nm and HR-LCMS searching for mass ion consistent with expected product. Experiments were performed in duplicate. The results are illustrated in FIG. 53.

The following experiments demonstrate the broad specificity any utility of the individual component enzymes.

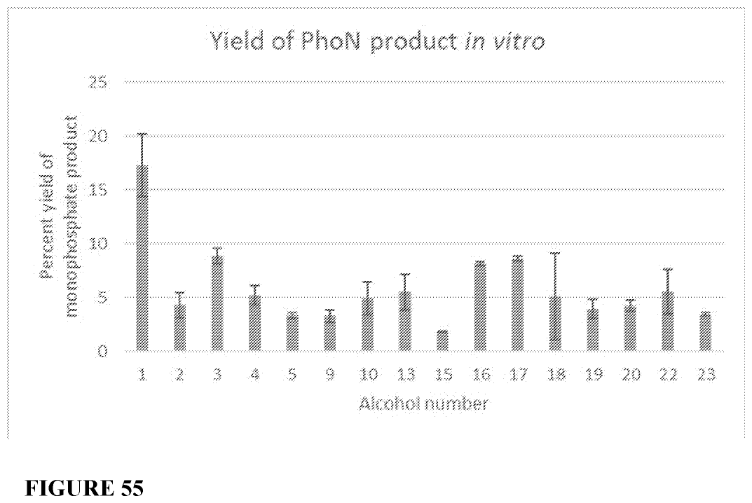

Broad substrate specificity of wild-type PhoN. Each commercially available alcohol (100 mM) was tested for activity with purified PhoN (20 μg/mL) and ATP (50 mM). The product mixtures were analyzed by high resolution liquid chromatography mass spectrometry (HR-LCMS) to quantify the conversion of alcohol to mono-phosphate. Quantification was achieved by the addition of an internal standard (either geranyl phosphate or cinnamyl monophosphate) at known fixed concentration. The results are illustrated in FIG. 54 and FIG. 55.

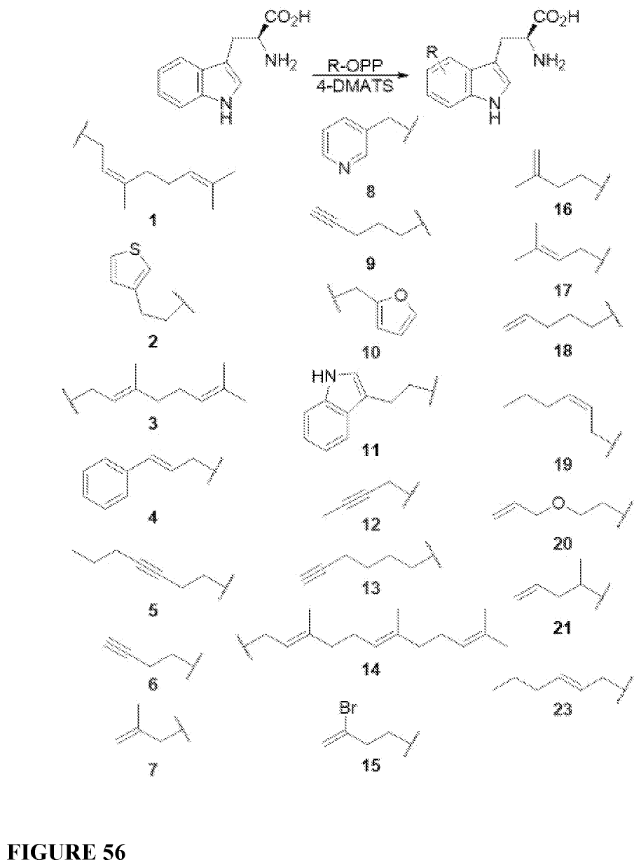

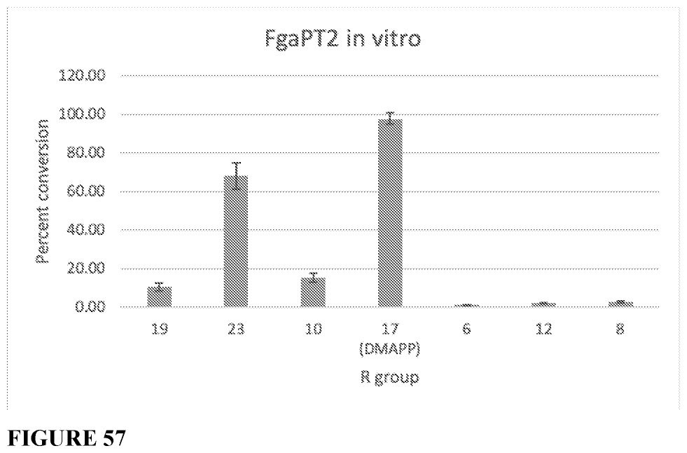

Broad substrate specificity of the wild-type prenyltransferase FgaPT2 in vitro. Each chemically synthesized pyrophosphate (3 mM) was tested for activity with purified FgaPT2 (200 μg/mL) and Trp (1 mM). The product mixtures were analyzed by HR-LCMS to identify the mass ions consistent with the expected alkylated product. The substrate (Trp) and product peak areas were determined by high performance liquid chromatography (HPLC) at a detection wavelength of 269 nm, and the conversion was calculated. The results are illustrated in FIG. 56 and FIG. 57.

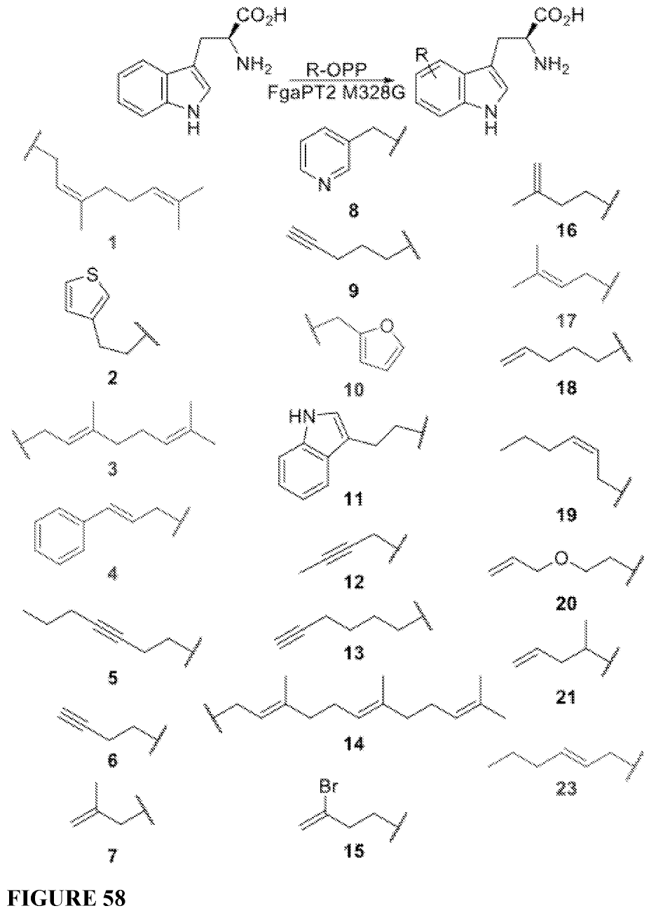

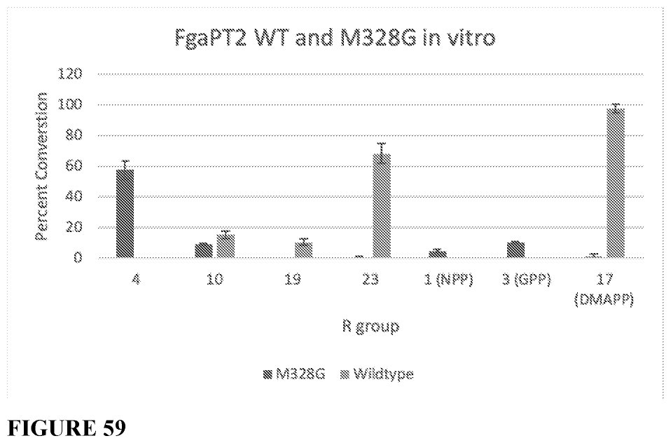

Altered substrate specificity of the prenyltransferase FgaPT2 mutant M328G in vitro. Each chemically synthesized pyrophosphate (3 mM) was tested for activity with purified FgaPT2 M328 (350 μg/mL) and Trp (1 mM). The product mixtures were analyzed by HPLC and confirmed by low-resolution liquid chromatography mass spectrometry (LR-LCMS) with mass ion consistent with expected product. The substrate (Trp) and product peak areas were determined by HPLC at a detection wavelength of 269 nm, and the conversion was calculated. The results are illustrated in FIG. 58 and FIG. 59.

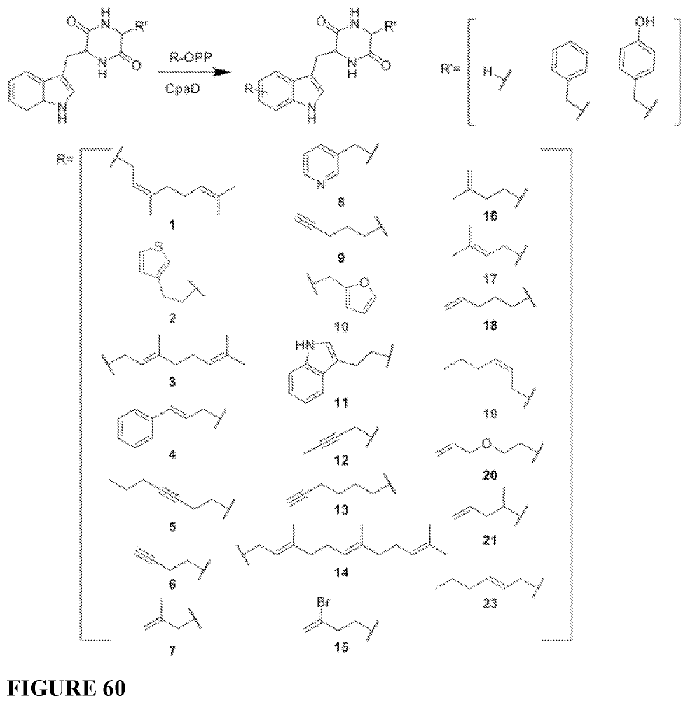

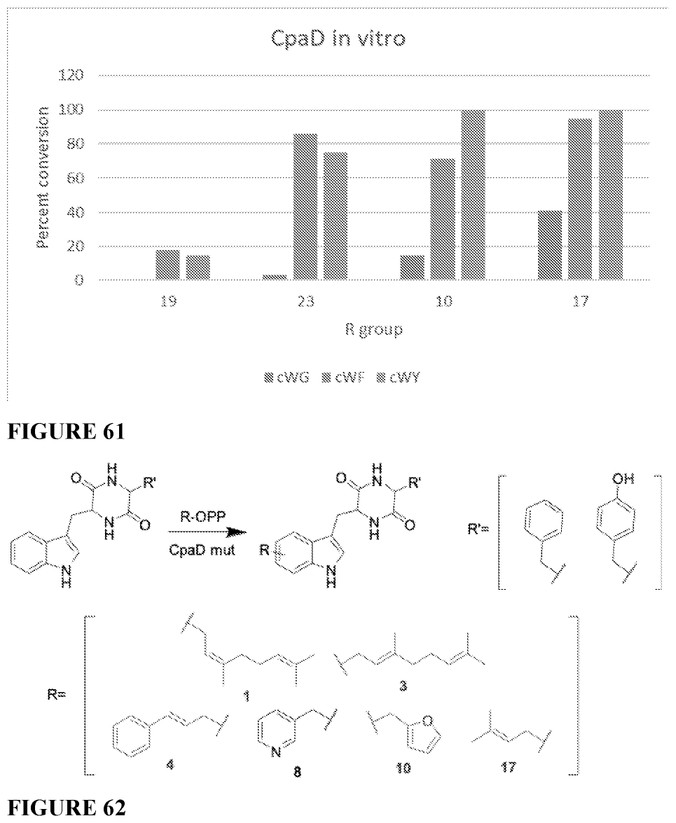

Broad substrate specificity of the prenyltransferase CpaD in vitro. Each chemically synthesized pyrophosphate (0.3 mM) and cyclic dipeptide (0.25 mM) were tested for activity with purified CpaD (1 mg/mL). The product mixtures were analyzed by HPLC and confirmed by LR-LCMS with mass ion consistent with expected product. The substrate (cyclic dipeptide) and product peak areas were determined by HPLC at a detection wavelength of 254 nm, and the conversion was calculated. The results are illustrated in FIG. 60 and FIG. 61.

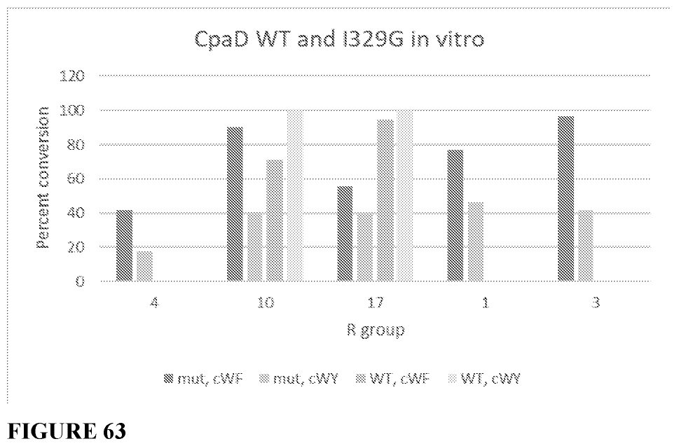

Altered substrate specificity of the prenyltransferase mutant CpaD 1329G in vitro. Each chemically synthesized pyrophosphate (2 mM) and cyclic dipeptide (1 mM) were tested for activity with purified CpaD I329G (1 mg/mL). The product mixtures were analyzed by HPLC and confirmed by LR-LCMS with mass ion consistent with expected product. The substrate (cyclic dipeptide) and product peak areas were determined by HPLC at a detection wavelength of 254 nm, and the conversion was calculated. The results are illustrated in FIG. 62 and FIG. 63.

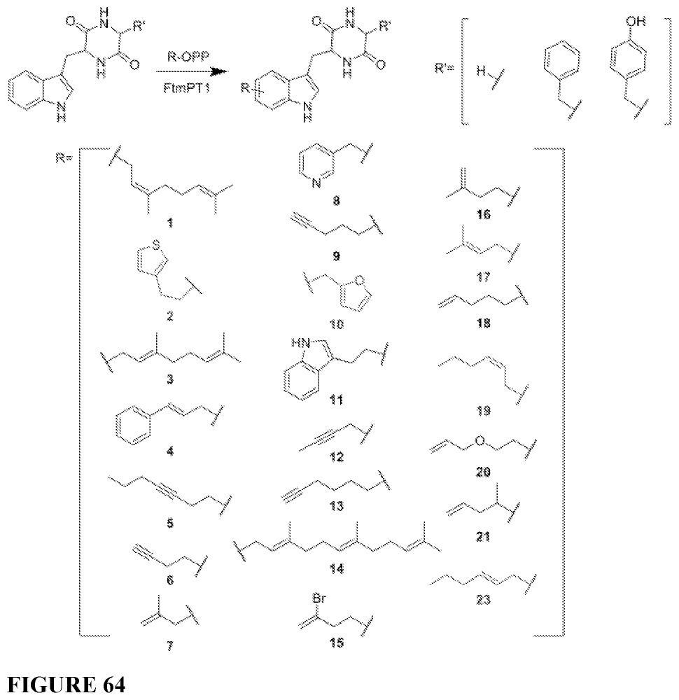

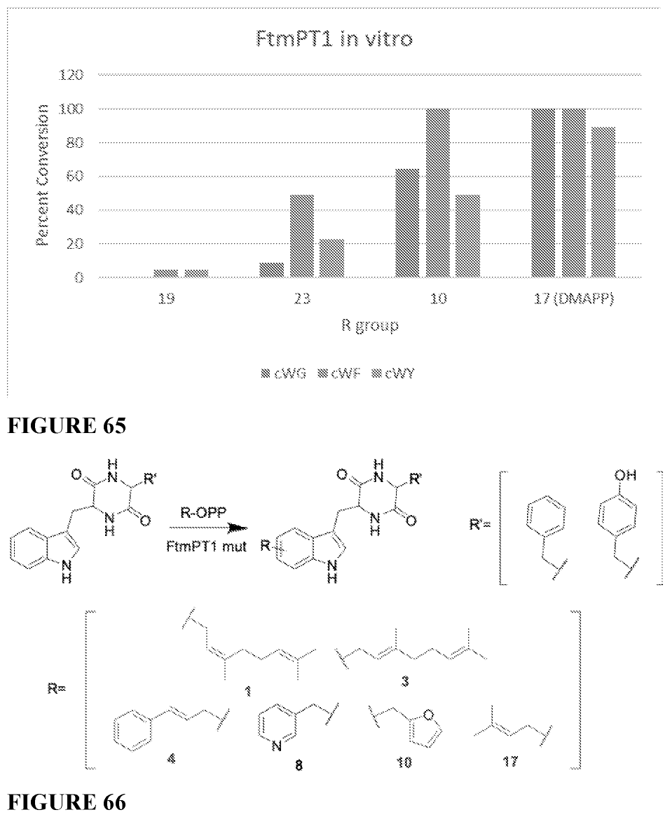

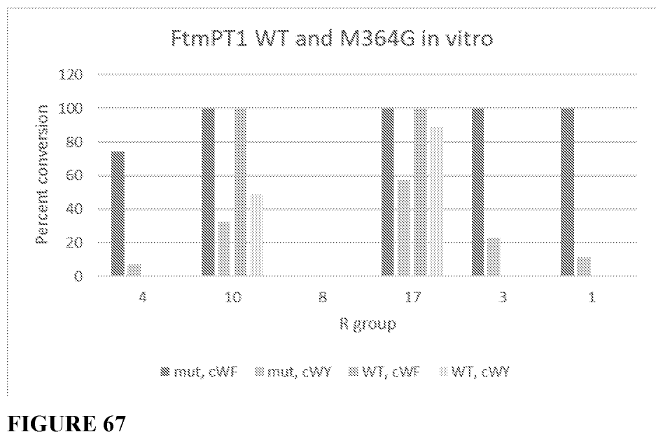

Broad substrate specificity of the prenyltransferase FtmPT1 in vitro. Each chemically synthesized pyrophosphate (2 mM) and cyclic dipeptide (1 mM) were tested for activity with purified FtmPT1 (100 mg/mL). The product mixtures were analyzed by HPLC and confirmed by LR-LCMS with mass ion consistent with expected product. The substrate (cyclic dipeptide) and product peak areas were determined by HPLC at a detection wavelength of 254 nm, and the conversion was calculated. The results are illustrated in FIG. 64 and FIG. 65.

Broad substrate specificity of the prenyltransferase mutant FtmPT1 M364G in vitro. Each chemically synthesized pyrophosphate (2 mM) and cyclic dipeptide (1 mM) were tested for activity with purified FtmPT1 (100 mg/mL). The product mixtures were analyzed by HPLC and confirmed by LR-LCMS with mass ion consistent with expected product. The substrate (cyclic dipeptide) and product peak areas were determined by HPLC at a detection wavelength of 254 nm, and the conversion was calculated. The results are illustrated in FIG. 64 and FIG. 65.

The compositions, systems, and methods of the appended claims are not limited in scope by the specific compositions, systems, and methods described herein, which are intended as illustrations of a few aspects of the claims. Any compositions, systems, and methods that are functionally equivalent are intended to fall within the scope of the claims. Various modifications of the compositions, systems, and methods in addition to those shown and described herein are intended to fall within the scope of the appended claims. Further, while only certain representative compositions, systems, and method steps disclosed herein are specifically described, other combinations of the components, compositions, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1-13. (canceled)

14. A method for synthesizing an isoprenoid subunit, the method comprising:
(i) providing a cell comprising genes encoding for (1) a phosphatase that exhibits bidirectional activity, and (2) a kinase;
(ii) incubating the cell in a fermentation broth with ATP and a primary alcohol defined by Formula I below 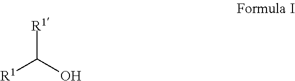
wherein
R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups;
R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
thereby generating the isoprenoid subunit.

15. The method of claim 14, wherein the phosphatase comprises a non-specific acid phosphatase.

16. The method of claim 14, wherein the phosphatase comprises PhoN.

17. The method of claim 14, wherein the kinase comprises a kinase that uses a phosphate acceptor.

18. The method of claim 14, wherein the kinase is chosen from a polyphosphate kinase, a phosphomevalonate kinase, a phosphomethylpyrimidine kinase, a farnesyl-diphosphate kinase, or a combination thereof.

19. The method of claim 14, wherein the kinase comprises isopentenyl phosphate kinase (IPK).

20. The method of claim 14, wherein the phosphatase, the kinase, or a combination thereof comprise a mutant enzyme engineered to increase substrate promiscuity, improve enzyme activity, increase enzyme specificity with respect to a particular substrate, or a combination thereof.

21. The method of claim 14, wherein the primary alcohol defined by Formula I is not one of the following

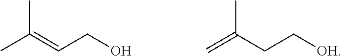

22. The method of claim 14, further comprising introducing the isoprenoid subunit into a natural or artificial isoprenoid biosynthetic pathway to synthesize an isoprenoid.

23. The method of claim 22, further comprising isolating the isoprenoid.

24-35. (canceled)

36. A method for synthesizing an isoprenoid subunit, the method comprising:
(i) providing a cell comprising genes encoding for a first kinase and a second kinase;
(ii) incubating the cell in a fermentation broth with ATP and a primary alcohol defined by Formula I below 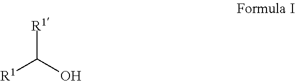
wherein
R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups
R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino,
with the proviso that the primary alcohol defined by Formula I is not one of the following 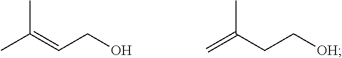
thereby generating the isoprenoid subunit.

37-55. (canceled)

56. A method for synthesizing an isoprenoid subunit, the method comprising:
(i) incubating a cell in a fermentation broth with ATP and a primary alcohol defined by Formula I below 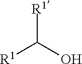
wherein
R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups
R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino,
thereby generating the isoprenoid subunit defined by Formula III below 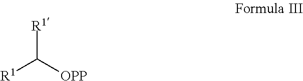
wherein R¹, R^{1′}, and R^{X} are as defined above with respect to Formula I and PP represents a pyrophosphate group;
wherein the cell comprises a gene encoding for a phosphotransferase that can catalyze both a first phosphorylation and a second phosphorylation of the primary alcohol defined by Formula I to generate the isoprenoid subunit.

57-74. (canceled)

75. A method for synthesizing an isoprenoid subunit, the method comprising:
(i) providing a cell comprising genes encoding for a first kinase and a second kinase, wherein the first kinase, the second kinase, or a combination thereof comprise a mutant enzyme engineered to increase substrate promiscuity, improve enzyme activity, increase enzyme specificity with respect to a particular substrate, or a combination thereof;
(ii) incubating the cell in a fermentation broth with ATP and a primary alcohol defined by Formula I below 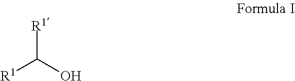
wherein
R¹ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups
R^{1′} is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
thereby generating the isoprenoid subunit.

76-83. (canceled)

84. An isoprenoid defined by Formula IV below 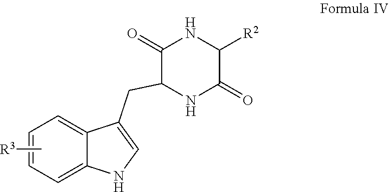
wherein
R² is selected from the group consisting of hydrogen, C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups
R³ is selected from the group consisting of C₁₋₁₀ alkyl, C₁₋₁₀ heteroalkyl, C₂₋₁₀ alkenyl, C₂₋₁₀ heteroalkenyl, C₂₋₁₀ alkynyl, C₂₋₁₀ heteroalkynyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, C₃₋₁₀ cycloalkyl-C₁₋₄ heteroalkylene, 4-10 membered heterocycloalkyl-C₁₋₄ alkylene, 4-10 membered heterocycloalkyl-C₁₋₄ heteroalkylene, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

85. The isoprenoid of claim 84, wherein R² is hydrogen.

86. The isoprenoid of claim 84, wherein R² is selected from the group consisting of 6-10 membered aryl, 5-10 membered heteroaryl, 6-10 membered aryl-C₁₋₄ alkylene, 6-10 membered aryl-C₁₋₄ heteroalkylene, 5-10 membered heteroaryl-C₁₋₄ alkylene, and 5-10 membered heteroaryl-C₁₋₄ heteroalkylene, each optionally substituted with 1, 2, 3, or 4 independently selected R^{X} groups.

87. The isoprenoid of claim 86, wherein R² comprises 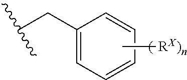
wherein n is 0, 1, or 2 and R^{X} is as defined above with respect to Formula IV.

88. The isoprenoid of claim 84, wherein R³ is not one of the following

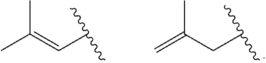

89. The isoprenoid of claim 84, wherein R³ is one of the following:

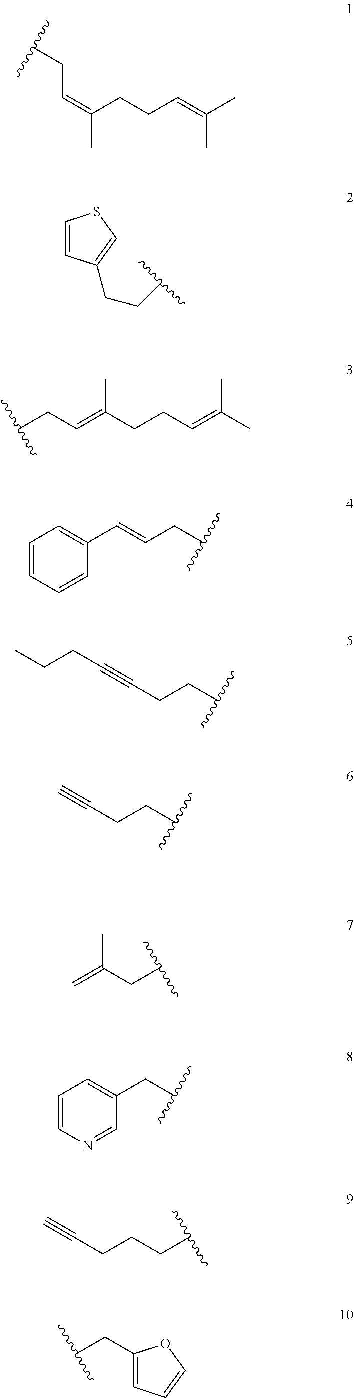

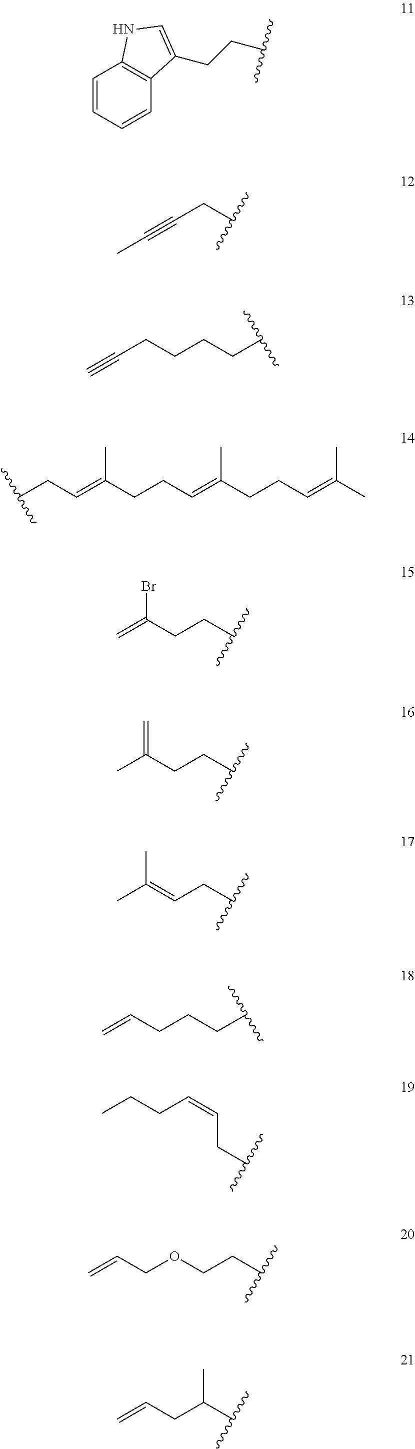

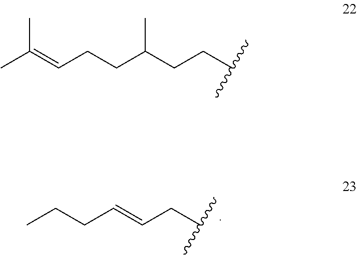

90-93. (canceled)


SEQUENCE LISTING (49 sequences)

SEQ ID NO: 1 - DNA, 750 bp, Artificial Sequence
  SYNTHETIC CONSTRUCT
atggatccgttcaccatgatgatcctgaagattggcggcagcgtgattaccgataagagcgcatatcgcaccgcacgcacctacgccattcgtagcatcgtgaaagtgctgagcggcattgaagatctggtgtgcgtggtgcatggcggtggtagctttggccacatcaaggcgatggagtttggtctgccgggtccgaaaaatccgcgtagcagcatcggctacagcatcgtgcatcgcgacatggaaaacctggacctgatggtgatcgacgcaatgatcgagatgggtatgcgcccgattagcgtgccgattagcgccctgcgttatgatggtcgctttgactacaccccgctgatccgctatattgatgcaggcttcgtgccggtgagctatggcgacgtgtatatcaaggacgaacatagctatggcatctacagcggcgacgatattatggccgatatggccgaactgctgaagccggatgtggccgtgttcctgaccgatgtggatggcatctatagcaaggacccgaaacgcaatccggatgccgtgctgctgcgcgacatcgatacaaacatcaccttcgatcgcgtgcagaacgatgtgaccggcggcattggcaagaaattcgaaagcatggttaaaatgaaaagtagcgttaaaaatggtgtgtacctgattaatggcaatcacccggagcgcattggtgacatcggcaaggagagcttcatcggtaccgtgattcgc

SEQ ID NO: 2 - DNA, 411 bp, Artificial Sequence
  SYNTHETIC CONSTRUCT
atgccgatggatctgcgcgacaacaaacagagccagaagaaatggaaaaaccgcaccctgaccagcagcctggaatttgccctgaccggcatttttaccgccttcaaagaagaacgcaacatgaaaaaacacgccgtgagcgcactgctggccgtgattgccggtctggtgttcaaagtgagcgtgatcgagtggctgtttctgctgctgagcatcttcctggtgatcaccttcgagatcgtgaacagtgccatcgagaatgtggtggatctggccagcgactatcacttcagcatgctggccaaaaacgccaaagatatggccgccggtgccgtgctggttattagcggttttgccgccctgaccggcctgattatttttctgctgaaaatttggtttctgctgtttcat

SEQ ID NO: 3 - DNA, 411 bp, Artificial Sequence; identical to SEQ ID NO: 2
  SYNTHETIC CONSTRUCT

SEQ ID NO: 4 - DNA, 1377 bp, Artificial Sequence
  SYNTHETIC CONSTRUCT
atgaaagccgcaaacgcaagcagcgcagaagcatatcgcgtgctgagccgcgccttccgctttgacaacgaggatcagaaactgtggtggcacagcaccgcaccgatgtttgcaaagatgctggaaaccgccaactatactaccccgtgccagtaccagtatctgatcacctataaggagtgtgtgatcccgagcttaggctgctatcctaccaatagcgcacctcgctggctgagtatcctgacccgttacggtaccccgtttgagctgagcctgaactgcagcaacagcatcgtgcgctacacctttgagccgattaaccagcataccggcaccgataaagacccgttcaacacccatgccatttgggaaagtctgcagcatctgctgccgttagagaagagcatcgatctggaatggttccgccacttcaaacacgacctgaccctgaatagcgaagaaagcgcctttctggcccataacgatcgcctggtgggcggtaccatccgcacccagaacaaactggcactggacctgaaagacggtcgcttcgccctgaaaacctatatctacccggccctgaaagccgtggtgaccggcaagaccattcacgagctggtgttcggtagcgttcgtcgcttagccgttcgcgaaccgcgcattctgccgccgctgaacatgctggaagagtatatccgtagccgcggcagcaaaagcaccgcaagtccgcgtctggttagctgtgacctgaccagcccggcaaaaagccgcatcaaaatctacctgctggagcagatggtgagcctggaagccatggaagatctgtggacattaggcggccgtcgccgtgatgccagcaccctggaaggtctgagcctggttcgtgaactgtgggatctgattcagctgagcccgggcctgaaaagctaccctgccccgtatctgccgctgggtgtgattcctgacgagcgtttaccgctgatggccaattttaccctgcaccagaacgatccggtgccggaaccgcaggtgtactttacaaccttcggcatgaatgacatggccgtggccgatgcactgaccaccttttttgagcgtcgcggctggagtgaaatggcacgcacctatgaaaccaccctgaagagctactacccgcatgccgaccatgataaactgaactatttacatgcctacatcagctttagctatcgcgatcgcaccccgtatctgagtgtgtacctgcagagcttcgaaacaggtgactgggccgtggcaaacctgagcgaaagcaaggtgaaatgccaggatgccgcctgtcagccgacagcactgccgcctgatctgagtaaaaccggcgtgtactacagcggcctgcat

SEQ ID NO: 5 - DNA, 36 bp, Artificial Sequence
  SYNTHETIC CONSTRUCT
catgggccggccactttccctctctcttccgacttg

SEQ ID NO: 6 - DNA, 38 bp, Artificial Sequence
  SYNTHETIC CONSTRUCT
catgctcgagttattggaagttttctagaacctgttcg

SEQ ID NO: 7 - DNA, 35 bp, Artificial Sequence
  SYNTHETIC CONSTRUCT
catgcatatggttcgtgccttcaaaattaaagttc

SEQ ID NO: 8 - DNA, 33 bp, Artificial Sequence
  SYNTHETIC CONSTRUCT
catgctcgagttatcattgctgttcgacgctag

SEQ ID NO: 9 - DNA, 33 bp, Artificial Sequence
  SYNTHETIC CONSTRUCT
catgcatatgcctgatatgaagctttttgctgg

SEQ ID NO: 10 - DNA, 34 bp, Artificial Sequence
  SYNTHETIC CONSTRUCT
catgctcgagttagtgttcgaacatggcagagat

SEQ ID NO: 11 - DNA, 31 bp, Artificial Sequence
  SYNTHETIC CONSTRUCT
catgcatatggttaaagtttatgccccggct

SEQ ID NO: 12 - DNA, 33 bp, Artificial Sequence
  SYNTHETIC CONSTRUCT
catgctcgagttagttttccagtactcgtgcgc

SEQ ID NO: 13 - DNA, 27 bp, Artificial Sequence
  SYNTHETIC CONSTRUCT
catgcatatggcgcacgacgaacaatg

SEQ ID NO: 14 - DNA, 35 bp, Artificial Sequence
  SYNTHETIC CONSTRUCT
catgctcgagttatcattttgcatatagcccctcc

SEQ ID NO: 15 - DNA, 28 bp, Artificial Sequence
  SYNTHETIC CONSTRUCT
catgcatatgcaagtcgacctgctgggt

SEQ ID NO: 16 - DNA, 30 bp, Artificial Sequence
  SYNTHETIC CONSTRUCT
catgctcgagttaaggaggtgcaggcatga

SEQ ID NO: 17 - DNA, 29 bp, Artificial Sequence
  SYNTHETIC CONSTRUCT
cagtagatctcagcgatatgcactcgctg

SEQ ID NO: 18 - DNA, 33 bp, Artificial Sequence
  SYNTHETIC CONSTRUCT
cagtctcgagttaaaagaacacgacatacaccg

SEQ ID NO: 19 - DNA, 33 bp, Artificial Sequence
  SYNTHETIC CONSTRUCT
catgcatatggccaataataccactggattcac

SEQ ID NO: 20 - DNA, 32 bp, Artificial Sequence
  SYNTHETIC CONSTRUCT
catgctcgagttatccaaaatgcgaccataac

SEQ ID NO: 21 - DNA, 35 bp, Artificial Sequence
  SYNTHETIC CONSTRUCT
catgcatatgactgaaaaaaaatatatcgttgcgc

SEQ ID NO: 22 - DNA, 32 bp, Artificial Sequence
  SYNTHETIC CONSTRUCT
catgctcgagttattcgtcgtgttcttcccac

SEQ ID NO: 23 - DNA, 28 bp, Artificial Sequence
  SYNTHETIC CONSTRUCT
ccatggcgatgcggacacagtggccctc

SEQ ID NO: 24 - DNA, 29 bp, Artificial Sequence
  SYNTHETIC CONSTRUCT
ctcgagttaagcatggctctgtgcaatgg

SEQ ID NO: 25 - DNA, 41 bp, Artificial Sequence
  SYNTHETIC CONSTRUCT
cgatggatccgatggcaactactagtactactacaaagctc

SEQ ID NO: 26 - DNA, 34 bp, Artificial Sequence
  SYNTHETIC CONSTRUCT
cgatgcggccgcttagaagagtaagaatccggcc

SEQ ID NO: 27 - DNA, 31 bp, Artificial Sequence
  SYNTHETIC CONSTRUCT
gtacagatcttatgcaaacggaacacgtcat

SEQ ID NO: 28 - DNA, 29 bp, Artificial Sequence
  SYNTHETIC CONSTRUCT
gtacctcgagttaattgtgctgcgcgaaa

SEQ ID NO: 29 - DNA, 31 bp, Artificial Sequence
  SYNTHETIC CONSTRUCT
gtacccatggcgatggactttccgcagcaac

SEQ ID NO: 30 - DNA, 40 bp, Artificial Sequence
  SYNTHETIC CONSTRUCT
gtacgcggccgcttatttattacgctggatgatgtagtcc

SEQ ID NO: 31 - DNA, 37 bp, Artificial Sequence
  SYNTHETIC CONSTRUCT
gtacccatgggcatgaaaccaactacggtaattggtg

SEQ ID NO: 32 - DNA, 34 bp, Artificial Sequence
  SYNTHETIC CONSTRUCT
gtacaagcttttaaatcagatcctccagcatcaa

SEQ ID NO: 33 - DNA, 31 bp, Artificial Sequence
  SYNTHETIC CONSTRUCT
gtacagatctcatggcagttggctcgaaaag

SEQ ID NO: 34 - DNA, 26 bp, Artificial Sequence
  SYNTHETIC CONSTRUCT
gtacctcgagttagagcgggcgctgc

SEQ ID NO: 35 - DNA, 35 bp, Artificial Sequence
  SYNTHETIC CONSTRUCT
gtaccatatgatagatccgttcaccatgatgatcc

SEQ ID NO: 36 - DNA, 31 bp, Artificial Sequence
  SYNTHETIC CONSTRUCT
gtacctcgagttagcgaatcacggtaccgat

SEQ ID NO: 37 - DNA, 32 bp, Artificial Sequence
  SYNTHETIC CONSTRUCT
gtaccatatggatccgttcaccatgatgatcc

SEQ ID NO: 38 - DNA, 31 bp, Artificial Sequence; identical to SEQ ID NO: 36
  SYNTHETIC CONSTRUCT

SEQ ID NO: 39 - DNA, 32 bp, Artificial Sequence
  SYNTHETIC CONSTRUCT
ctagggatccgatgaaacgtcagctgtttacc

SEQ ID NO: 40 - DNA, 37 bp, Artificial Sequence
  SYNTHETIC CONSTRUCT
ctaggcggccgcttattttttctgattattggcgaat

SEQ ID NO: 41 - DNA, 26 bp, Artificial Sequence
  SYNTHETIC CONSTRUCT
ataggatccgatgccgatggatctgc

SEQ ID NO: 42 - DNA, 37 bp, Artificial Sequence
  SYNTHETIC CONSTRUCT
cgatgcggccgcttaatgaaacagcagaaaccaaatt

SEQ ID NO: 43 - DNA, 22 bp, Artificial Sequence
  SYNTHETIC CONSTRUCT
atacatatgaaagccgcaaacg

SEQ ID NO: 44 - DNA, 20 bp, Artificial Sequence
  SYNTHETIC CONSTRUCT
tataagcttatgcaggccgc

SEQ ID NO: 45 - DNA, 27 bp, Artificial Sequence
  SYNTHETIC CONSTRUCT
tataggatccgatgaaagccgcaaacg

SEQ ID NO: 46 - DNA, 21 bp, Artificial Sequence
  SYNTHETIC CONSTRUCT
atataagcttatgcaggccgc

SEQ ID NO: 47 - DNA, 20 bp, Artificial Sequence
  SYNTHETIC CONSTRUCT
ttgtacacggccgcataatc

SEQ ID NO: 48 - DNA, 16 bp, Artificial Sequence
  SYNTHETIC CONSTRUCT
atgcgtccggcgtaga

SEQ ID NO: 49 - DNA, 750 bp, Artificial Sequence; identical to SEQ ID NO: 1
  SYNTHETIC CONSTRUCT